(19) United States
(12) Patent Application Publication
(10) Pub. No.: US 2024/0316122 A1
(43) Pub. Date: Sep. 26, 2024

(54) METHODS OF TREATING CANCER USING RECOMBINANT MICROORGANISMS EXPRESSING A STING AGONIST

(71) Applicant: Synlogic Operating Company, Inc., Cambridge, MA (US)

(72) Inventors: Anna Sokolovska, Somerville, MA (US); Aoife Brennan, Dover, MA (US)

(21) Appl. No.: 18/271,714

(22) PCT Filed: Jan. 11, 2022
(86) PCT No.: PCT/US2022/012030

§ 371 Date: Jul. 11, 2023

Related U.S. Application Data
(60) Provisional application No. 63/277,213, filed on Nov. 9, 2021.
(60) Provisional application No. 63/173,661, filed on Apr. 12, 2021.
(60) Provisional application No. 63/166,503, filed on Mar. 26, 2021.
(60) Provisional application No. 63/135,934, filed on Jan. 11, 2021.

(51) Int. Cl. A61K 35/741; A61K 39/00; A61K 39/395; C07K 16/28; C12N 1/20; C12N 9/48; C12R 1/19

(52) U.S. Cl. CPC A61K 35/741; A61K 39/3955; C07K 16/2827; C12N 1/205; C12N 9/485; C12Y 304/16004; A61K 2039/505; A61K 2039/545; C12R 2001/19

(57) ABSTRACT

Therapies comprising a recombinant microorganism expressing a STING agonist, and methods of modulating and treating cancers are disclosed.

### RELATED APPLICATIONS

The instant application claims priority to U.S. Provisional Application No. 63/277,213, filed on Nov. 9, 2021; U.S. Provisional Application No. 63/173,661, filed Apr. 12, 2021; U.S. Provisional Application No. 63/166,503, filed Mar. 26, 2021; and U.S. Provisional Application No. 63/135,934, filed Jan. 11, 2021, entire contents of each of which are expressly incorporated by reference herein in their entireties.

### SEQUENCE LISTING

The instant application contains a Sequence Listing which has been submitted electronically in ASCII format and is hereby incorporated by reference in its entirety. Said ASCII copy, created on Jan. 3, 2022 is named 126046-06020_SL.txt and is 1,796,837 bytes in size.

### BACKGROUND

Current cancer therapies typically employ the use of immunotherapy, surgery, chemotherapy, radiation therapy, or some combination thereof (American Cancer Society). While these drugs have shown great benefits to cancer patients, many cancers remain difficult to treat using conventional therapies. Currently, many conventional cancer therapies are administered systemically and adversely affect healthy tissues, resulting in significant side effects. For example, many cancer therapies focus on activating the immune system to boost the patient's anti-tumor response (Kong et al., 2014). However, despite such therapies, the microenvironment surrounding tumors remains highly immune suppressive. In addition, systemic altered immunoregulation provokes immune dysfunction, including the onset of opportunistic autoimmune disorders and immune-related adverse events.

Major efforts have been made over the past few decades to develop cytotoxic drugs that specifically target cancer cells. In recent years there has been a paradigm shift in oncology in which the clinical problem of cancer is considered not only to be the accumulation of genetic abnormalities in cancer cells but also the tolerance of these abnormal cells by the immune system. Consequently, recent anti-cancer therapies have been designed specifically to target the immune system rather than cancer cells. Such therapies aim to reverse the cancer immunotolerance and stimulate an effective antitumor immune response. For example, current immunotherapies include immunostimulatory molecules that are pattern recognition receptor (PRR) agonists or immunostimulatory monoclonal antibodies that target various immune cell populations that infiltrate the tumor microenvironment. However, despite their immune-targeted design, these therapies have been developed clinically as if they were conventional anticancer drugs, relying on systemic administration of the immunotherapeutic (e.g., intravenous infusions every 2-3 weeks). As a result, many current immunotherapies suffer from toxicity due to a high dosage requirement and also often result in an undesired autoimmune response or other immune-related adverse events.

Thus, there is an unmet need for effective cancer therapies that are able to target poorly vascularized, hypoxic tumor regions specifically target cancerous cells, while minimally affecting normal tissues and boost the immune systems to fight the tumors, including avoiding or reversing the cancer immunotolerance.

### SUMMARY

The present disclosure is based on a first-in-human clinical trial data and provides compositions, methods, and uses of a bacterium that selectively targets tumors and tumor cells and achieves specific immune modulation results in human subjects. The use of the bacterium is safe and provides targeted and local delivery of therapeutic compositions.

Specifically, the present disclosure provides a method of treating a human subject with cancer, by administering to the human subject a recombinant microorganism capable of expressing a stimulator of interferon gene (STING) agonist. In one embodiment, a level of one or more interferon-stimulated genes, one or more T cell function genes, one or more chemokine genes, one or more cytokine genes, and/or one or more serum cytokines are upregulated in the human subject after administration, as compared to a control level of the one or more interferon-stimulated genes, one or more T cell function genes, one or more chemokine genes, one or more cytokine genes, and/or one or more serum cytokines.

In some embodiments, the one or more interferon-stimulated genes are selected from the group consisting of ISG15, IFIT1, and IFIT2. In some embodiments, the one or more T cell function genes are selected from the group consisting of GZMA, CD4, CD8, and PD-L2. In some embodiments, the one or more chemokines and/or cytokine genes are selected from the group consisting of CXCL9, CXCL10, TNFRS1B, and TNFSF10.

In some embodiments, the level of the one or more interferon-stimulated genes, one or more T cell function genes, one or more chemokine genes, and/or one or more cytokine genes in the subject after administration is upregulated by about 2-fold, about 3-fold, or about 4-fold, as compared to the control level.

In some embodiments, the one or more serum cytokines are selected from the group consisting of IL-6, TNFα, IFNγ, and IL-1Ra.

In some embodiments, the level of the one or more serum cytokines is about 2-fold, about 5-fold, about 10-fold, or about 20-fold higher than the control level of the one or more serum cytokines.

In some embodiments, the control level is (i) a level of the one or more interferon-stimulated genes, one or more T cell function genes, one or more chemokine genes, one or more cytokine genes, and/or one or more serum cytokines in the human subject prior to administration, or (ii) a level of the one or more interferon-stimulated genes, one or more T cell function genes, one or more chemokine genes, one or more cytokine genes, and/or one or more serum cytokines in a control human subject or population of control human subjects who has not been administered the recombinant microorganism. In one embodiment, the control subject or population of control human subjects have cancer or a tumor.

In some embodiments, the recombinant microorganism is administered to the subject at a suitable dose. In some embodiments, the recombinant microorganism is administered to the subject at a dose of about 1×10⁶, about 3×10⁶, about 1×10⁷, about 3×10⁷, about 1×10⁸, about 3×10⁸, or about 1×10⁹ live cells. In some embodiments, the suitable dose may be selected from about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, and from about 3×10⁸ to about 1×10⁹ live cells.

In some embodiments, the recombinant microorganism is administered to a subject once weekly, once every two weeks, or once every three weeks. In some embodiments, the recombinant microorganism is administered to a subject once weekly. In some embodiments, the recombinant microorganism is administered to a subject once every three weeks.

In one embodiment, the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months. In one embodiment, the recombinant microorganism is administered for at least 24 months.

In some embodiments, the cancer is melanoma, liposarcoma, sarcoma, small cell lung cancer, squamous cell carcinoma, or chondrosarcoma.

In some embodiments, the method further comprises administering an additional therapeutic agent. In one embodiment, the additional therapeutic agent is an anti-PD1 antibody or an anti-PDL1 antibody. In some embodiments, the anti-PDL1 antibody is atezolizumab.

In some embodiments, the STING agonist is c-diAMP, c-GAMP, or c-diGMP. In some embodiments, the STING agonist is c-diAMP.

In some embodiments, the recombinant microorganism expresses at least one non-native gene sequence encoding an enzyme capable of producing the STING agonist.

In some embodiments, the at least one non-native gene sequence is dacA or cGAS. In one embodiment, the dacA sequence has at least 90%, at least 91%, at least 92%, at least 93%, at least 94%, at least 95%, at least 96%, at least 97%, at least 98%, at least 99% identity to, comprises, or consists of SEQ ID NO: 1210. In one embodiment the dacA gene sequence encodes a protein that comprises a sequence that has at least 90%, at least 91%, at least 92%, at least 93%, at least 94%, at least 95%, at least 96%, at least 97%, at least 98%, at least 99% identity to, comprises, or consists of SEQ ID NO: 1209.

In some embodiments, the at least one non-native gene sequence is integrated into a chromosome of the microorganism or is present on a plasmid in the microorganism.

In some embodiments, the at least one non-native gene sequence encoding the enzyme capable of producing the STING agonist is operably linked to an inducible promoter or a constitutive promoter. In some embodiments, the at least one non-native gene sequence encoding the enzyme capable of producing the STING agonist is operably linked to an inducible promoter. In some embodiments, the at least one non-native gene sequence encoding the enzyme capable of producing the STING agonist is operably linked to a constitutive promoter. In one embodiment, the non-native gene sequence is operably linked to an inducible promoter and has at least 90%, at least 91%, at least 92%, at least 93%, at least 94%, at least 95%, at least 96%, at least 97%, at least 98%, at least 99% identity to, comprises, or consists of SEQ ID NO: 1284.

In some embodiments, inducible promoter is induced by low-oxygen or anaerobic conditions, by the hypoxic environment of a tumor, or temperature.

In some embodiments, the recombinant microorganism further comprises one or more auxotrophies. In some embodiments, the recombinant microorganism is an auxotroph in dapA, thyA, or both dapA and thyA. In one embodiment, the recombinant microorganism further comprises one or more phage deletions.

In some embodiments, the recombinant microorganism is non-pathogenic to the subject. In some embodiments, the recombinant microorganism is a recombinant bacteria. In some embodiments, the recombinant microorganism is E. coli. In some embodiments, the recombinant microorganism is Escherichia coli Nissle.

In some embodiments, the method further comprises selecting a subject who would benefit from an increase in the level of the one or more interferon-stimulated genes, one or more T cell function genes, one or more chemokine genes, one or more cytokine genes, and/or one or more serum cytokines.

In some embodiments, method further comprises isolating a sample from the subject after administration and measuring the level of the one or more interferon-stimulated genes, one or more T cell function genes, one or more chemokine genes, one or more cytokine genes, and/or one or more serum cytokines.

In some embodiments, the method further comprises isolating a sample from the subject prior to administration and measuring the level of the one or more interferon-stimulated genes, one or more T cell function genes, one or more chemokine genes, one or more cytokine genes, and/or one or more serum cytokines.

In some embodiments, the sample is a tumor biopsy and/or a serum sample.

In some embodiments, the administering is intratumoral injection.

In another aspect, the present disclosure provides a method of increasing the number of T cells present in a tumor or a cancer in a human subject, the method comprising administering to the human subject a recombinant microorganism capable of expressing a stimulator of interferon gene (STING) agonist, wherein the number of T cells present in the tumor or cancer is increased by at least about 25%, about 50%, about 75%, about 100%, about 1.5-fold, about 2-fold, about 2.5-fold or about 3-fold after administration of the recombinant microorganism as compared to a number of T cells present in the tumor or cancer prior to administration of the recombinant microorganism.

In one embodiment, the T cells present in the tumor after administration are CD4+, CD8+, CD11c+/MHCII+, CD11c+/PD-L1+, CD4+/Ki67+, CD8+/Ki67+, CD8+/GrnzB+, CD11c+/CD8+/MHCII+, CD11c+, MHCII+, PD-LI+, or a combination thereof.

In one embodiments, the recombinant microorganism is administered to the subject at a suitable dose. In one embodiment, the recombinant microorganism is administered to the subject at a dose of about 1×10⁶, about 3×10⁶, about 1×10⁷, about 3×10⁷, about 1×10⁸, about 3×10⁸, or about 1×10′ live cells. In one embodiment, the recombinant microorganism is administered to the subject at a dose of about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, or from about 3×10⁸ to about 1×10⁹ live cells. In one embodiment, the recombinant microorganism is administered to the subject once weekly, once every two weeks, or once every three weeks. In one embodiment, the recombinant microorganism is administered to the subject once weekly. In one embodiment, the recombinant microorganism is administered to the subject once every three weeks.

In one embodiment, the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months. In one embodiment, the recombinant microorganism is administered for at least 24 months. In one embodiment, the recombinant microorganism is administered for 6 weeks to 3 months, 6 weeks to 6 months, 6 weeks to 1 year, 6 weeks to 18 months, or 6 weeks to 2 years.

In one embodiment, wherein the STING agonist is c-diAMP, c-GAMP, or c-diGMP. In one embodiment, the recombinant microorganism expresses at least one non-native gene sequence encoding an enzyme capable of producing the STING agonist. In one embodiment, the at least one non-native gene sequence is dacA or cGAS.

In one embodiment, the cancer or tumor is melanoma, liposarcoma, sarcoma, small cell lung cancer, squamous cell carcinoma, or chondrosarcoma. In one embodiment, the tumor or cancer is stable or regressing after administration.

In another aspect, the present disclosure provides a method of treating a human subject having a tumor or a cancer, the method comprising: selecting a human subject that would benefit from an increase in the number of T cells present in the tumor, and administering to the human subject a recombinant microorganism capable of expressing a stimulator of interferon gene (STING) agonist, wherein the number of T cells present in the tumor is increased by at least about 25%, about 50%, about 75%, about 100%, about 1.5-fold, about 2-fold, about 2.5-fold or about 3-fold after administration of the recombinant microorganism as compared to a number of T cells present in the tumor prior to administration of the recombinant microorganism.

In one embodiment, the T cells present in the tumor after administration are CD4+, CD8+, CD11c+/MHCII+, CD11c+/PD-L1+, CD4+/Ki67+, CD8+/Ki67+, CD8+/GrnzB+, CD11c+/CD8+/MHCII+, CD11c+, MHCII+, PD-LI+, or a combination thereof.

In one embodiments, the recombinant microorganism is administered to the subject at a suitable dose. In one embodiment, the recombinant microorganism is administered to the subject at a dose of about 1×10⁶, about 3×10⁶, about 1×10⁷, about 3×10⁷, about 1×10⁸, about 3×10⁸, or about 1×10′ live cells. In one embodiment, the recombinant microorganism is administered to the subject at a dose of about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, or from about 3×10⁸ to about 1×10⁹ live cells. In one embodiment, the recombinant microorganism is administered to the subject once weekly, once every two weeks, or once every three weeks. In one embodiment, the recombinant microorganism is administered to the subject once weekly. In one embodiment, the recombinant microorganism is administered to the subject once every three weeks.

In one embodiment, the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months. In one embodiment, the recombinant microorganism is administered for at least 24 months. In one embodiment, the recombinant microorganism is administered for 6 weeks to 3 months, 6 weeks to 6 months, 6 weeks to 1 year, 6 weeks to 18 months, or 6 weeks to 2 years.

In one embodiment, wherein the STING agonist is c-diAMP, c-GAMP, or c-diGMP. In one embodiment, the recombinant microorganism expresses at least one non-native gene sequence encoding an enzyme capable of producing the STING agonist. In one embodiment, the at least one non-native gene sequence is dacA or cGAS.

In one embodiment, the cancer or tumor is melanoma, liposarcoma, sarcoma, small cell lung cancer, squamous cell carcinoma, or chondrosarcoma.

In one embodiment, the tumor or cancer is stable or regressing after administration.

In another aspect, the present disclosure provides a method of increasing survival of a human subject suffering from cancer or having a tumor, the method comprising administering to the human subject a recombinant microorganism capable of expressing a stimulator of interferon gene (STING) agonist, wherein the number of T cells present in the tumor or cancer is increased by at least about 25%, about 50%, about 75%, about 100%, about 1.5-fold, about 2-fold, about 2.5-fold or about 3-fold after administration of the recombinant microorganism as compared to a number of T cells present in the tumor or cancer prior to administration of the recombinant microorganism, wherein the cancer and/or tumor is stable or regressing after administration.

In one embodiment, the T cells present in the tumor after administration are CD4+, CD8+, CD11c+/MHCII+, CD11c+/PD-L1+, CD4+/Ki67+, CD8+/Ki67+, CD8+/GrnzB+, CD11c+/CD8+/MHCII+, CD11c+, MHCII+, PD-LI+, or a combination thereof.

In one embodiments, the recombinant microorganism is administered to the subject at a suitable dose. In one embodiment, the recombinant microorganism is administered to the subject at a dose of about 1×10⁶, about 3×10⁶, about 1×10⁷, about 3×10⁷, about 1×10⁸, about 3×10⁸, or about 1×10′ live cells. In one embodiment, the recombinant microorganism is administered to the subject at a dose of about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, or from about 3×10⁸ to about 1×10⁹ live cells. In one embodiment, the recombinant microorganism is administered to the subject once weekly, once every two weeks, or once every three weeks. In one embodiment, the recombinant microorganism is administered to the subject once weekly. In one embodiment, the recombinant microorganism is administered to the subject once every three weeks.

In one embodiment, the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months. In one embodiment, the recombinant microorganism is administered for at least 24 months. In one embodiment, the recombinant microorganism is administered for 6 weeks to 3 months, 6 weeks to 6 months, 6 weeks to 1 year, 6 weeks to 18 months, or 6 weeks to 2 years.

In one embodiment, wherein the STING agonist is c-diAMP, c-GAMP, or c-diGMP. In one embodiment, the recombinant microorganism expresses at least one non-native gene sequence encoding an enzyme capable of producing the STING agonist. In one embodiment, the at least one non-native gene sequence is dacA or cGAS.

In one embodiment, the cancer or tumor is melanoma, liposarcoma, sarcoma, small cell lung cancer, squamous cell carcinoma, or chondrosarcoma.

In one embodiment, the tumor responds to treatment resulting in a decrease of at least 5%, at least 10%, at least 20%, at least 25%, or at least 30% of a Response Evaluation Criteria in Solid Tumours (RECIST) when compared to the RECIST prior to treatment. In one embodiment, the RECIST decreases at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% when compared to the RECIST prior to treatment.

In one embodiment, the method further comprising administering an additional therapeutic agent, optionally wherein the additional therapeutic agent is an anti-PD1 antibody or an anti-PDL1 antibody. In one embodiment, the anti-PDL1 antibody is atezolizumab.

In any of the above embodiments, the microorganism may be SYNB1891. In one embodiment, SYNB1891 comprises the genotype ΔthyA-ΔdapA-ΔΦ-P_{furS}-dacA.

### BRIEF DESCRIPTION OF THE FIGURES

FIG. 1A depicts IL-6 levels in the blood pre-injection and 6 hr and 24 hr after intratumoral injection of SYNB1891 at doses 1×10⁶, 3×10⁶, and 1×10⁷ live cells.

FIG. 1B depicts TNF-α levels in the blood pre-injection and 6 hr and 24 hr after intratumoral injection of SYNB1891 at doses 1×10⁶, 3×10⁶, and 1×10⁷ live cells.

FIG. 1C depicts IFNγ levels in the blood pre-injection and 6 hr and 24 hr after intratumoral injection of SYNB1891 at doses 1×10⁶, 3×10⁶, and 1×10⁷ live cells.

FIG. 1D depicts IL-1Ra levels in the blood pre-injection and 6 hr and 24 hr after intratumoral injection of SYNB1891 at doses 1×10⁶, 3×10⁶, and 1×10⁷ live cells.

FIG. 2A depicts a heatmap of gene expression in treated tumors. Fold change over baseline. Tumors were injected with SYNB1891 at doses 1×10⁶, 3×10⁶, and 1×10⁷ live cells. Baseline tumor at day 1, injected tumor a day 22 with 3 doses. 100-002: melanoma; 200-003: liposarcoma; 200-004: sarcoma; 600-002: small cell lung cancer; 100-600: squamous cell carcinoma; 100-005: chondrosarcoma.

FIG. 2B depicts fold change of gene expression in FIG. 2A.

FIG. 3A depicts a heatmap of expression of chemokine genes in treated tumors. Fold change over baseline. Tumors treated as in FIG. 2A.

FIG. 3B depicts a heatmap of expression of cytokine genes in treated tumors. Fold change over baseline. Tumors treated as in FIG. 2A.

FIG. 3C depicts a heatmap of expression of T cell function genes in treated tumors. Fold change over baseline. Tumors treated as in FIG. 2A.

FIG. 4A depicts five most upregulated genes across six patients in categories: interferon-stimulated genes (ISGs), chemokines, cytokines, TLRs (innate immunity), and T cell function. Fold change over baseline.

FIG. 4B depicts most upregulated genes across six patients in ISGs, chemokines and cytokines, and T cell functions. Fold change over baseline.

FIG. 5A depicts sum of measurements and percent change over the progression of 12 cycles of treatment with 1×10⁶ cells. Patient 100-002 (metastatic melanoma previously treated with Nivolumab) showed stable disease at 7 months followed by progressive disease.

FIG. 5B depicts heat map of expression of interferon-stimulated genes, cytokine/chemokine genes, and T cell function genes in treated tumor of the patient from FIG. 5A.

FIG. 6A depicts sum of measurements and percent change over the progression of 10 cycles of treatment with 1×10⁷ cells. Patient 600-002 (small cell lung cancer previously treated with Pembrolizumab) showed stable disease at 4.5 months.

FIG. 6B depicts heat map of expression of interferon-stimulated genes, antigen processing genes, and T cell function genes in treated tumor of the patient from FIG. 6A.

FIGS. 7A-7H depict multiplex immunofluorescence staining (IF) for tumor cores. Patient 100-002 (vulvar melanoma): baseline (FIG. 7A); SYNB1891 (FIG. 7B); patient 600-002 (small cell lung cancer); baseline (FIG. 7C); SYNB1891 (FIG. 7D); patient 200-003 (liposarcoma); baseline (FIG. 7E); SYNB1891 (FIG. 7F); and patient 100-005 (chondrosarcoma of the bone): baseline (FIG. 7G); SYNB1891 (FIG. 7H). DAPI (nucleus) in blue, CD4+ cells in green, CD8+ cells in red.

FIG. 8A depicts a heat map of marker positive cells in T cell compartment and APC compartment in a tumor.

FIG. 8B depicts a heat map of cell phenotype in T cell compartment and APC compartment in a tumor.

FIGS. 9A-9K depict quantification of cells with specific marker in a tumor area based on multiplex immunofluorescence staining (e.g., FIGS. 7A-7H). CD4+ cells (FIG. 9A), CD8+ cells (FIG. 9B), CD11c/MHCII+ cells (FIG. 9C), CD11c/PDL-1+ cells (FIG. 9D), CD4+/Ki67+ cells (FIG. 9E), CD8+/Ki67+ cells (FIG. 9F), CD8+/GrnzB+ cells (FIG. 9G), CD11c+/CD8+/MHCII+ cells (FIG. 9H), CD11c+ cells (FIG. 9I), MHCII+ cells (FIG. 9J), and PD-LI+ cells (FIG. 9K).

FIG. 10 graphically depicts the SYNB1891 strain. Abbreviations: ΔΦ=deletion of the prophage sequence endogenous to E. coli Nissle; ATP=adenosine triphosphate; dacA or DacA=diadenylate cyclase gene or enzyme; ΔdapA=deletion of dapA gene leading to diaminopimelate auxotrophy; LPS=lipopolysaccharide or endotoxin; Pfnrs=fumarate and nitrate reductase regulator promoter; ΔthyA=deletion of thyA gene leading to thymidine auxotrophy.

FIG. 11 depicts a summary of the Phase I clinical trial design including ARM 1, monotherapy, and ARM 2, combination with atezolizumab.

FIGS. 12A, 12B, 12C, and 12D depict serum cytokine levels in patients at baseline, 6 hours, and 24 hours after injection of SYNB1891 at 1×10⁶, 3×10⁶, 1×10⁷, 3×10⁸, 1×10⁸, and 3×10⁸ live cells. Cytokines IL-6 (FIG. 12A), TNFα (FIG. 12B), IFNγ (FIG. 12C), and IL-1RA were measured.

FIG. 13A depicts baseline gene expression levels across different tumor types. Data were normalized to the highest and lowest expressed gene in each row. The majority of injected tumors had low levels of inflammatory gene expression.

FIG. 13B depicts fold change in gene expression over baseline in injected tumors. Fold changes in gene expression after one cycle of SYNB1819 treatment relative to baseline samples as determined by Nanostring.

FIG. 14 depicts change from baseline in gene expression for the 5 most upregulated genes across 5 different gene categories. Data are shown for the same 12 patients depicted in FIGS. 13A and 13B. Each symbol depicts the data for a single patient. ISG=interferon stimulated genes.

### DETAILED DESCRIPTION

Certain tumors are particularly difficult to manage using conventional therapies. Hypoxia is a characteristic feature of solid tumors, wherein cancerous cells are present at very low oxygen concentrations. Regions of hypoxia often surround necrotic tissues and develop as solid forms of cancer outgrow their vasculature. When the vascular supply is unable to meet the metabolic demands of the tumor, the tumor's microenvironment becomes oxygen deficient. Multiple areas within tumors contain <1% oxygen, compared to 3-15% oxygen in normal tissues (Vaupel and Hockel, 1995), and avascular regions may constitute 25-75% of the tumor mass (Dang et al., 2001). Approximately 95% of tumors are hypoxic to some degree (Huang et al., 2004). Systemically delivered anticancer agents rely on tumor vasculature for delivery, however, poor vascularization impedes the oxygen supply to rapidly dividing cells, rendering them less sensitive to therapeutics targeting cellular proliferation in poorly vascularized, hypoxic tumor regions. Radiotherapy fails to kill hypoxic cells because oxygen is a required effector of radiation-induced cell death. Hypoxic cells are up to three times more resistant to radiation therapy than cells with normal oxygen levels (Bettegowda et al., 2003; Tiecher, 1995; Wachsberger et al., 2003). For all of these reasons, nonresectable, locally advanced tumors are particularly difficult to manage using conventional therapies.

In addition to the challenges associated with targeting a hypoxic environment, therapies that specifically target and destroy cancers must recognize differences between normal and malignant tissues, including genetic alterations and pathophysiological changes that lead to heterogeneous masses with areas of hypoxia and necrosis.

The disclosure relates to microorganisms, e.g., bacteria, pharmaceutical compositions thereof, and methods of modulating or treating cancer. In certain embodiments, the bacteria are delivered locally to the tumor cells. In certain embodiments, the bacteria are capable of targeting cancerous cells. In certain embodiments, the bacteria are capable of targeting cancerous cells, particularly in low-oxygen conditions, such as in hypoxic tumor environments.

This disclosure relates to compositions and therapeutic methods for the local and tumor-specific delivery of immune modulators in order to treat cancers. In certain aspects, the disclosure relates to microorganisms that are capable of targeting cancerous cells and, in some embodiments, the microorganism is administered in combination with one or more effector molecules e.g., immune modulators, such as any of the effector molecules provided herein. In contrast to existing conventional therapies, the hypoxic areas of tumors offer a perfect niche for the growth of anaerobic bacteria, the use of which offers an opportunity for eradication of advanced local tumors in a precise manner, sparing surrounding well-vascularized, normoxic tissue.

In some aspects, the disclosure provides delivery of a microorganism to tumor cells or the tumor microenvironment. In some aspects, the disclosure relates to a microorganism that is delivered locally, e.g., via local intra-tumoral administration, in combination with one or more effector molecules, e.g., immune initiators and/or immune sustainers. In some aspects, the compositions and methods disclosed herein may be used to deliver one or more effector molecules, e.g., immune initiators and/or immune sustainers selectively to tumor cells, thereby reducing systemic cytotoxicity or systemic immune dysfunction, e.g., the onset of an autoimmune event or other immune-related adverse event.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

The generation of immunity to cancer is a potentially self-propagating cyclic process which has been referred to as the “Cancer-Immunity Cycle” (Chen and Mellman, Oncology Meets Immunology: The Cancer-Immunity Cycle; Immunity (2013) 39:1-10), and which can lead to the broadening and amplification of the T cell response. The cycle is counteracted by inhibitory factors that lead to immune regulatory feedback mechanisms at various steps of the cycle and which can halt the development or limit the immunity.

The cycle essentially comprises a series of steps which need to occur for an anticancer immune response to be successfully mounted. The cycle includes steps, which must occur for the immune response to be initiated and a second series of events which must occur subsequently, in order for the immune response to be sustained (i.e., allowed to progress and expand and not dampened). These steps have been referred to as the “Cancer-Immunity Cycle” (Chen and Mellman, 2013), and are essentially as follows:
1. Release (oncolysis) and/or acquisition of tumor cell contents; Tumor cells break open and spill their contents, resulting in the release of neoantigens, which are taken up by antigen presenting cells (dendritic cells and macrophages for processing). Alternatively, antigen presenting cells may actively phagocytose tumors cells directly.
2. Activation of antigen presenting cells (APC) (dendritic cells and macrophages); In addition to the first step described above, the next step must involve release of proinflammatory cytokines or generation of proinflammatory cytokines as a result of release of DAMPs or PAMPs from the dying tumor cells to result in antigen presenting cell activation and subsequently an anticancer T cell response. Antigen presenting cell activation is critical to avoid peripheral tolerance to tumor derived antigens. If properly activated, antigen presenting cells present the previously internalized antigens on their surface in the context of MHCI and MHCII molecules alongside the proper co-stimulatory signals (CD80/86, cytokines, etc.) to prime and activate T cells.
3. Priming and Activation of T cells: Antigen presentation by DCs and macrophages causes the priming and activation of effector T cell responses against the cancer-specific antigens, which are seen as “foreign” by the immune system. This step is critical to the strength and breadth of the anti-cancer immune response, by determining quantity and quality of T effector cells and contribution of T regulatory cells. Additionally, proper priming of T cells can result in superior memory T cell formation and long lived immunity.
4. Trafficking and Infiltration: Next, the activated effector T cells must traffic to the tumor and infiltrate the tumor.
5. Recognition of cancer cells by T cells and T cell support, and augmentation and expansion of effector T cell responses: Once arrived at the tumor site, the T cells can recognize and bind to cancer cells via their T cell receptors (TCR), which specifically bind to their cognate antigen presented within the context of MHC molecules on the cancer cells, and subsequently kill the target cancer cell. Killing of the cancer cell releases tumor associated antigens through lysis of tumor cells, and the cycle re-initiates, thereby increasing the volume of the response in subsequent rounds of the cycle. Antigen recognition by either MHC-I or MHC-II restricted T cells can result in additional effector functions, such as the release of chemokines and effector cytokines, further potentiating a robust antitumor response.
6. Overcoming immune suppression: Finally, overcoming certain deficiencies in the immune response to the cancer and/or overcoming the defense strategy of the cancer, i.e., overcoming the breaks that the cancer employs in fighting the immune response, can be viewed as another critical step in the cycle. In some cases, even though T cell priming and activation has occurred, other immunosuppressive cell subsets are actively recruited and activated to the tumor microenvironment, i.e., regulatory T cells or myeloid derived suppressor cells. In other cases, T cells may not receive the right signals to properly home to tumors or may be actively excluded from infiltrating the tumor. Finally, certain mechanisms in the tumor microenvironment exist, which are capable of suppressing or repressing the effector cells that are produced as a result of the cycle. Such resistance mechanisms co-opt immune-inhibitory pathways, often referred to as immune checkpoints, which normally mediate immune tolerance and mitigate cancer tissue damage (see e.g., Pardoll (2012), The blockade of immune checkpoints in cancer immunotherapy; Nature Reviews Cancer volume 12, pages 252-264).

One important immune-checkpoint receptor is cytotoxic T-lymphocyte-associated antigen 4 (CTLA4), which down modulates the amplitude of T cell activation. Some immune-checkpoint receptors, such as programmed cell death protein 1 (PD1), limit T cell effector functions within tissues. By upregulating ligands for PD1, tumor cells and antigen presenting cells block antitumor immune responses in the tumor microenvironment. Multiple additional immune-checkpoint receptors and ligands, some of which are selectively upregulated in various types of tumor cells, are prime targets for blockade, particularly in combination with approaches that enhance the initiation or activation of antitumor immune responses.

Therapies have been developed to promote and support progression through the cancer-immunity cycle at one or more of the 6 steps. These therapies can be broadly classified as therapies that promote initiation of the immune response and therapies that help sustain the immune response.

As used herein the term “immune initiation” or “initiating the immune response” refers to advancement through the steps which lead to the generation and establishment of an immune response. For example, these steps could include the first three steps of the cancer immunity cycle described above, i.e., the process of antigen acquisition (step (1)), activation of dendritic cells and macrophages (step (2)), and/or the priming and activation of T cells (step (3)).

As used herein the term “immune sustenance” or “sustaining the immune response” refers to the advancement through steps which ensure the immune response is broadened and strengthened over time and which prevent dampening or suppression of the immune response. For example, these steps could include steps 4 through 6 of the cycle described, i.e., T cell trafficking and tumor infiltration, recognition of cancer cells though TCRs, and overcoming immune suppression, i.e., depletion or inhibition of T regulatory cells and preventing the establishment of other active suppression of the effector response.

Accordingly, in some embodiments, the compositions are capable of modulating, e.g., advancing the cancer immunity cycle by modulating, e.g., activating, promoting supporting, one or more of the steps in the cycle. In some embodiments, the compositions are capable of modulating, e.g., promoting, steps that modulate, e.g., intensify, the initiation of the immune response. In some embodiments, the compositions are capable of modulating, e.g., boosting, certain steps within the cycle that enhance sustenance of the immune response. In some embodiments, the compositions are capable of modulating, e.g., intensifying, the initiation of the immune response and modulating, e.g., enhancing, sustenance of the immune response.

Accordingly, in some embodiments, the one or more effector molecules, e.g., immune modulators, modulate, e.g., intensify the initiation of the immune response. Accordingly, in some embodiments, the one or more effector molecules, e.g., immune modulators, modulate, e.g., enhance, sustenance of the immune response. Accordingly, in some embodiments, the one or more effector molecules, e.g., immune modulators, modulate, e.g., intensify, the initiation of the immune response and the one or more one or more effector molecules, e.g., immune modulators, modulate, e.g., enhance, sustenance of the immune response.

An “effector”, “effector substance” or “effector molecule” refers to one or more molecules, therapeutic substances, or drugs of interest. In one embodiment, the “effector” is administered in combination with a microorganism, e.g., bacteria, and administered before, at the same time as, or after, the administration of the microorganism. In another embodiment, a microorganism described herein is administered in combination with at least two effectors and administered before, at the same time as, or after, the administration of the microorganism.

A non-limiting example of such effector or effector molecules are “immune modulators,” which include immune sustainers and/or immune initiators as described herein. In some embodiments, the composition comprises two or more effector molecules or immune modulators. In some embodiments, the composition comprises three, four, five, six, seven, eight, nine, or ten effector molecules or immune modulators. In some embodiments, the effector molecule or immune modulator is a therapeutic molecule that is useful for modulating or treating a cancer.

In some embodiments, the effector or immune modulator is a therapeutic molecule. In some embodiments, the effector molecule or immune modulator may be a nucleic acid molecule that mediates RNA interference, microRNA response or inhibition, TLR response, antisense gene regulation, target protein binding (aptamer or decoy oligos), or gene editing, such as CRISPR interference. Other types of effectors and immune modulators are described and listed herein.

Non-limiting examples of effector molecules and/or immune modulators include immune checkpoint inhibitors (e.g., CTLA-4 antibodies, PD-1 antibodies, PDL-1 antibodies), cytotoxic agents (e.g., Cly A, FASL, TRAIL, TNFα), immunostimulatory cytokines and co-stimulatory molecules (e.g., OX40 antibody or OX40L, CD28, ICOS, CCL21, IL-2, IL-18, IL-15, IL-12, IFN-gamma, IL-21, TNFs, GM-CSF), antigens and antibodies (e.g., tumor antigens, neoantigens, CtxB-PSA fusion protein, CPV-OmpA fusion protein, NY-ESO-1 tumor antigen, RAF1, antibodies against immune suppressor molecules, anti-VEGF, Anti-CXR4/CXCL12, anti-GLP1, anti-GLP2, anti-galectini, anti-galectin3, anti-Tie2, anti-CD47, antibodies against immune checkpoints, antibodies against immunosuppressive cytokines and chemokines), DNA transfer vectors (e.g., endostatin, thrombospondin-1, TRAIL, SMAC, Stat3, Bcl2, FLT3L, GM-CSF, IL-12, AFP, VEGFR2), and enzymes (e.g., E. coli CD, HSV-TK), immune stimulatory metabolites and biosynthetic pathway enzymes that produce them (STING agonists, e.g., c-di-AMP, 3′3′-cGAMP, and 2′3′-cGAMP; arginine, tryptophan).

Effectors may also include enzymes or other polypeptides (such as transporters or regulatory proteins) or other modifications (such as inactivation of certain endogenous genes, e.g., auxotrophies), which result in catabolism of immune suppressive or tumor growth promoting metabolites, such as kynurenine, adenosine and ammonia.

Immune modulators include, inter alia, immune initiators and immune sustainers.

As used herein, the term “immune initiator” or “initiator” refers to a class of effectors or molecules, e.g., immune modulators, or substances. Immune initiators may modulate, e.g., intensify or enhance, one or more steps of the cancer immunity cycle, including (1) lysis of tumor cells (oncolysis); (2) activation of APCs (dendritic cells and macrophages); and/or (3) priming and activation of T cells. In one embodiment, an immune initiator may be administered in combination with a microorganism of the disclosure. For example, a microorganism described herein is administered in combination with at least one immune initiator but administered before, at the same time as, or after, the administration of the microorganism. In other embodiments, a microorganism described herein is administered in combination with at least one immune initiator and at least one immune sustainer, but administered before, at the same time as, or after, the administration of the microorganism. Non-limiting examples of such immune initiators are described in further detail herein.

In some embodiments, an immune initiator is a therapeutic molecule. Non-limiting examples of such therapeutic molecules are described herein and include, but are not limited to, cytokines, chemokines, single chain antibodies (agonistic or antagonistic), ligands (agonistic or antagonistic), co-stimulatory receptors/ligands and the like. In another embodiment, an immune initiator is a STING agonist. In another embodiment, an immune initiator is arginine. In another embodiment, an immune initiator is at least one enzyme of a catabolic pathway. Non-limiting examples of such catabolic pathways are described herein and include, but are not limited to, enzymes involved in the catabolism of a harmful metabolite. In another embodiment, an immune initiator is at least one molecule produced by at least one enzyme of a biosynthetic pathway. In another embodiment, an immune initiator is a metabolic converter. In other embodiments, the immune initiator may be a nucleic acid molecule that mediates RNA interference, microRNA response or inhibition, TLR response, antisense gene regulation, target protein binding (aptamer or decoy oligos), gene editing, such as CRISPR interference.

In specific embodiments, the one or more immune initiators, modulate, e.g., intensify, one or more of steps (1) lysis of tumor cells and/or uptake of tumor antigens, (2), activation of APCs and/or (3) priming and activation of T cells. In some embodiments, the one or more immune initiators modulate, e.g., intensify, one or more of steps (1) lysis of tumor cells and/or uptake of tumor antigens, (2) activation of APCs and/or (3) priming and activation of T cells. In some embodiments, the one or more immune initiators, modulate, e.g., intensify, one or more of steps (1) oncolysis and/or uptake of tumor antigens, (2) activation of APCs and/or (3) priming and activation of T cells. Any immune initiator may be combined with one or more additional same or different immune initiator(s), which modulate the same or a different step in the cancer immunity cycle.

In one embodiment, the one or more immune initiators which modulate oncolysis or tumor antigen uptake (step (1)). Non-limiting examples of immune initiators which modulate antigen acquisition are described herein and known in the art and include but are not limited to lytic peptides, CD47 blocking antibodies, SIRP-alpha and variants, TNFα, IFN-γ and 5FU. In one embodiment, the one or more immune initiators which modulate activation of APCs (step (2)). Non-limiting examples of immune initiators modulate activation of APCs are described herein and known in the art and include but are not limited to Toll-like receptor agonists, STING agonists, CD40L, and GM-CSF. In one embodiment, the one or more immune initiators modulate, e.g., enhance, priming and activation of T cells (step (3)). Non-limiting examples of immune initiators which modulate, e.g., enhance, priming and activation of T cells are described herein and known in the art and include but are not limited to an anti-OX40 antibody, OXO40L, an anti-41BB antibody, 41BBL, an anti-GITR antibody, GITRL, anti-CD28 antibody, anti-CTLA4 antibody, anti-PD1 antibody, anti-PDL1 antibody, IL-15, and IL-12, etc.

As used herein the term “immune sustainer” or “sustainer” refers to a class of effectors or molecules, e.g., immune modulators, or substances. Immune sustainers may modulate, e.g., boost or enhance, one or more steps of the cancer immunity cycle, including (4) trafficking and infiltration; (5) recognition of cancer cells by T cells and T cell support; and/or (6) the ability to overcome immune suppression. In one embodiment, the immune sustainer may be administered in combination with a microorganism described herein. For example, a microorganism described herein is administered in combination with an immune sustainer but administered before, at the same time as, or after, the administration of the microorganism and/or the immune sustainer.

In some embodiments, the immune sustainer is a therapeutic molecule. Non-limiting examples of such therapeutic molecules are described herein and include cytokines, chemokines, single chain antibodies (agonistic or antagonistic), ligands (agonistic or antagonistic), and the like. In another embodiment, an immune sustainer is a therapeutic molecule produced by an enzyme. Non-limiting examples of such enzymes are described herein. In another embodiment, an immune sustainer is at least one enzyme of a biosynthetic pathway or a catabolic pathway. Non-limiting examples of such biosynthetic pathways are described herein and include, but are not limited to, enzymes involved in the production of arginine; and non-limiting examples of such catabolic pathways are described herein and include, but are not limited to, enzymes involved in the catalysis of kynurenine or enzymes involved in the catalysis of adenosine. In another embodiment, an immune sustainer is a metabolic converter. In other embodiments, the immune sustainer may be a nucleic acid molecule that mediates RNA interference, microRNA response or inhibition, TLR response, antisense gene regulation, target protein binding (aptamer or decoy oligos), gene editing, such as CRISPR interference.

In specific embodiments, the term “immune sustainer” may also refer to the reduction or elimination of a harmful molecule. In such instances, the term “immune sustainer” may also be used to refer to the one or more enzymes of the catabolic pathway which breaks down the harmful metabolite.

In some embodiments, the one or more immune sustainers modulate, e.g., boost, one or more of steps (4) T cell trafficking and infiltration, (5) recognition of cancer cells by T cells and/or T cell support and/or (6) the ability to overcome immune suppression. Any immune sustainer may be combined with one or more additional immune sustainer(s), which modulate the same or a different step. In some embodiments, the one or more immune sustainers, modulate, e.g., boost, one or more of steps (4) T cell trafficking and infiltration, (5) recognition of cancer cells by T cells and/or T cell support and/or (6) the ability to overcome immune suppression. In some embodiments, the one or more immune sustainers modulate, e.g., boost, one or more of steps (4) T cell trafficking and infiltration, (5) recognition of cancer cells by T cells and/or T cell support and/or (6) the ability to overcome immune suppression.

In one embodiment, the one or more immune sustainers modulate T cell trafficking and infiltration (step (4)). Non-limiting examples of immune sustainers which modulate T cell trafficking and infiltration are described herein and known in the art and include, but are not limited to, chemokines such as CXCL9 and CXCL10 or upstream activators which induce the expression of such cytokines. In one embodiment, the one or more immune sustainers modulate recognition of cancer cells by T cells and T cell support (step (5)). Non-limiting examples of immune sustainers which modulate recognition of cancer cells by T cells and T cell support are described herein and known in the art and include, but are not limited to, anti-PD1/PD-L1 antibodies (antagonistic), anti-CTLA-4 antibodies (antagonistic), kynurenine consumption, adenosine consumption, anti-OX40 antibodies (agonistic), anti-41BB antibodies (agonistic), and anti-GITR antibodies (agonistic). In one embodiment, the one or more immune sustainers modulate, e.g., enhance, the ability to overcome immune suppression (step (6)). Non-limiting examples of immune sustainers which modulate, e.g., enhance, the ability to overcome immune suppression are described herein and known in the art and include, but are not limited to, IL-15 and IL-12 and variants thereof.

Any one or more immune initiator(s) may be combined any one or more immune sustainer(s). Accordingly, in some embodiments, the one or more immune initiators modulate, e.g., intensify, one or more of steps (1) oncolysis, (2) activation of APCs and/or (3) priming and activation of T cells in combination with one or more immune sustainers, which modulate, e.g., boost, one or more of steps (4) T cell trafficking and infiltration, (5) recognition of cancer cells by T cells and/or T cell support and/or (6) the ability to overcome immune suppression.

In some embodiments, certain immune modulators act at multiple stages of the cancer immunity cycle, e.g., one or more stages of immune initiation, or one or more of immune sustenance, or at one or more stages of immune initiation and at one or more stages of immune sustenance.

As used herein a “metabolic conversion” refers to a chemical transformation which is the result of an enzyme-catalyzed reaction. The enzyme-catalyze reaction can be either biosynthetic or catabolic in nature.

As used herein, the term “metabolic converter” refers to one or more enzymes, which catalyze a chemical transformation, i.e., which consume, produce or convert a metabolite. In some embodiments, the term “metabolic converter” refers to the at least one molecule produced by the at least one enzyme of a biosynthetic pathway. A metabolic converter can consume a toxic or immunosuppressive metabolite or produce an anti-cancer metabolite, or both. Non-limiting examples of metabolic converters include kynurenine consumers, adenosine consumers, arginine producers and/or ammonia consumers, i.e., enzymes for the consumption of kynurenine or adenosine or for the production of arginine and/or consumption of ammonia. In one embodiment, a metabolic converter can be, for example, a human kynureninase enzyme (for example, EC 3.7.1.3). In another embodiment, a metabolic converter can be a nucleic acid, e.g., RNAi molecule (siRNA, miRNA, dsRNA), mRNA, antisense molecule, aptamer, or CRISPR/Cas 9 molecule, that increases or decreases endogenous expression of an enzyme(s) that catalyzes a chemical transformation, i.e., which consume, produce or convert a metabolite, in a tumor.

As used herein “wild-type” refers to an unmodified bacteria. For example, a wild-type bacteria has not been modified using genetic engineering. A wild-type bacteria, for example, has not been modified to express a non-native gene or to comprise an auxotrophy. In one embodiment, a wild-type bacteria is an E. coli Nissle bacteria.

A “bacteria chassis” or “chassis,” as used herein, refers to a bacteria that may comprise an auxotrophic modification, e.g., a mutation or deletion in dapA, thyA, or both, and/or deletion of a phage, and may stimulate an innate immune response; but the bacteria is not modified to comprise a non-native nucleic acid or gene or to express a non-native protein. In other words, a bacteria chassis refers to a bacteria that has not been modified to comprise a non-native immune modulator gene or to express a non-native immune modulator protein. In some embodiments, a chassis refers to a strain of Escherichia coli Nissle bacteria that may comprise an auxotrophic modification, e.g., a mutation or deletion in dapA, thyA, or both, and may stimulate an innate immune response, but is not modified to comprise a non-native gene or to express a non-native protein, e.g., has not been modified to comprise a non-native immune modulator nucleic acid or gene or to express a non-native immune modulator protein.

As used herein, “non-native” refers to a nucleic acid or a protein not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature.

As used herein, the term “endogenous gene” refers to a native gene in its natural location in the genome of an organism. As used herein, the term “transgene” refers to a gene that has been introduced into the host organism, e.g., host bacterial cell, genome.

As used herein, the term “partial regression” refers to an inhibition of growth of a tumor, and/or the regression of a tumor, e.g., in size, after administration of the microorganism(s) and/or immune modulator(s) to a subject having the tumor. In one embodiment, a “partial regression” may refer to a regression of a tumor, e.g., in size, by at least about 5%, at least about 10%, at least about 15%, at least about 20%, at least about 25%, at least about 30%, at least about 40%, at least about 50%, at least about 60%, at least about 70%, at least about 80%, or at least about 90%. In another embodiment, a “partial regression” may refer to a decrease in the size of a tumor by at least about 5%, at least about 10%, at least about 15%, at least about 20%, at least about 25%, at least about 30%, at least about 40%, at least about 50%, at least about 60%, at least about 70%, at least about 75%, at least about 80%, or at least about 90%. In one embodiment, “partial regression” refers to the regression of a tumor, e.g., in size, but wherein the tumor is still detectable in the subject.

As used herein, the term “complete regression” refers to a complete regression of a tumor, e.g., in size, after administration of the microorganism(s) and/or immune modulator(s) to the subject having the tumor. When “complete regression” occurs the tumor is undetectable in the subject.

As used herein, the term “percent response” refers to a percentage of subjects in a population of subjects who exhibit either a partial regression or a complete regression, as defined herein, after administration of a microorganism(s) and/or immune modulator(s). For example, in one embodiment, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of subjects in a population of subjects exhibit a partial response or a complete response.

As used herein, the term “stable disease” refers to a cancer or tumor that is neither growing nor shrinking. “Stable disease” also refers to a disease state where no new tumors have developed, and a cancer or tumor has not spread to any new region or area of the body, e.g., by metastasis.

“Intratumoral administration” is meant to include any and all means for microorganism delivery to the intratumoral site and is not limited to intratumoral injection means. Examples of delivery means for the microorganisms is discussed in detail herein.

“Cancer” or “cancerous” is used to refer to a physiological condition that is characterized by unregulated cell growth. In some embodiments, cancer refers to a tumor. “Tumor” is used to refer to any neoplastic cell growth or proliferation or any pre-cancerous or cancerous cell or tissue. A tumor may be malignant or benign. Types of cancer include, but are not limited to, adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer (e.g., small cell lung cancer, non-small cell lung cancer), lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma (e.g., liposarcoma, chondrosarcoma), skin cancer (e.g., basal cell carcinoma, melanoma, squamous cell carcinoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenstrom macroglobulinemia, and Wilms tumor. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute).

As used herein, “abscopal” and “abscopal effect” refers to an effect in which localized treatment of a tumor not only shrinks or otherwise affects the tumor being treated, but also shrinks or otherwise affects other tumors outside the scope of the localized treatment. In some embodiments, the bacteria may elicit an abscopal effect. In some embodiments, no abscopal effect is observed upon administration of the bacteria.

As used herein, “Response Evaluation Criteria in Solid Tumours” or “RECIST” refers to a standard way to measure how well a cancer patient responds to treatment. It is based on whether tumors shrink, stay the same, or get bigger. To use RECIST, there must be at least one tumor that can be measured on x-rays, CT scans, or MRI scans. The types of response a patient can have are a complete response (CR), a partial response (PR), progressive disease (PD), and stable disease (SD).

In any of these embodiments in which abscopal effect is observed, timing of tumor growth in a tumor of the same type which is distal to the administration site is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days of the same type relative to the tumor growth (tumor volume) in a naive animal or subject.

In any of these embodiments in which an abscopal effect is observed, timing of tumor growth as measured in tumor volume in a distal tumor of the same type is delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which abscopal effect is observed, timing of tumor growth as measured in tumor volume in a tumor distal to the administration site of the same type is delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1.4-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject. In this example, “tumor re-challenge” may also include metastasis formation which may occur in a subject at a certain stage of cancer progression.

Immunological memory represents an important aspect of the immune response in mammals. Memory responses form the basis for the effectiveness of vaccines against cancer cells. As used herein, the term “immune memory” or “immunological memory” refers to a state in which long-lived antigen-specific lymphocytes are available and are capable of rapidly mounting responses upon repeat exposure to a particular antigen. The importance of immunological memory in cancer immunotherapy is known, and the trafficking properties and long-lasting anti-tumor capacity of memory T cells play a crucial role in the control of malignant tumors and prevention of metastasis or reoccurrence. Immunological memory exists for both B lymphocytes and for T cells, and is now believed to exist in a large variety of other immune cells, including NK cells, macrophages, and monocytes. (see e.g., Farber et al., Immunological memory: lessons from the past and a look to the future (Nat. Rev. Immunol. (2016) 16: 124-128). Memory B cells are plasma cells that are able to produce antibodies for a long time. The memory B cell has already undergone clonal expansion and differentiation and affinity maturation, so it is able to divide multiple times faster and produce antibodies with much higher affinity. Memory T cells can be both CD4+ and CD8+. These memory T cells do not require further antigen stimulation to proliferate therefore they do not need a signal via MHC.

Immunological memory can, for example, be measured in an animal model by re-challenging the animal model upon achievement of complete regression upon treatment with the microorganism. The animal is then implanted with cancer cells from the cancer cell line and growth is monitored and compared to an age matched naïve animal of the same type which had not previously been exposed to the tumor. Such a tumor re-challenge is used to demonstrate systemic and long term immunity against tumor cells and may represent the ability to fight off future recurrence or metastasis formation. Such an experiment is described herein using the A20 tumor model in the Examples. Immunological memory would prevent or slow the reoccurrence of the tumor in the re-challenged animal relative to the naïve animal. On a cellular level, formation of immunological memory can be measured by expansion and/or persistence of tumor antigen specific memory or effector memory T cells.

In some embodiments, immunological memory is achieved in a subject upon administration of the compositions described herein. In some embodiments, immunological memory is achieved cancer patient upon administration of the compositions described herein.

In some embodiments, a complete response is achieved in a subject upon administration of the compositions described herein. In some embodiments, a complete response is achieved in a cancer patient upon administration of the compositions described herein.

In some embodiments, a complete remission is achieved in a subject upon administration of the compositions described herein. In some embodiments, a complete remission is achieved in a cancer patient upon administration of the compositions described herein.

In some embodiments, a partial response is achieved in a subject upon administration of the compositions described herein. In some embodiments, a partial response is achieved in a cancer patient upon administration of the compositions described herein.

In some embodiments, stable disease is achieved in a subject upon administration of the compositions described herein. In some embodiments, a partial response is achieved in a cancer patient upon administration of the compositions described herein.

In some embodiments, a subset of subjects within a group achieves a partial or complete response upon administration of the compositions described herein. In some embodiments, a subset of patients within a group achieve a partial or complete response upon administration of the compositions described herein.

In any of these embodiments in which immunological memory is observed, timing of tumor growth is delayed by at least about 0 to 2 days, at least about 2 to 4 days, at least about 4 to 6 days, at least about 6 to 8 days, at least about 8 to 10 days, at least about 10 to 12 days, at least about 12 to 14 days, at least about 14 to 16 days, at least about 16 to 18 days, at least about 18 to 20 days, at least about 20 to 25 days, at least about 25 to 30 days, at least about 30 to 35 days in a tumor re-challenge relative to the tumor growth (tumor volume) in a naive animal or subject.

In any of these embodiments in which immunological memory is observed, timing of tumor growth as measured in tumor volume delayed by at least about 0 to 2 weeks, at least about 2 to 4 weeks, at least about 4 to 6 weeks, at least about 6 to 8 weeks, at least about 8 to 10 weeks, at least about 10 to 12 weeks, at least about 12 to 14 weeks, at least about 14 to 16 weeks, at least about 16 to 18 weeks, at least about 18 to 20 weeks, at least about 20 to 25 weeks, at least about 25 to 30 weeks, at least about 30 to 35 weeks, at least about 35 to 40 weeks, at least about 40 to 45 weeks, at least about 45 to 50 weeks, at least about 50 to 55 weeks, at least about 55 to 60 weeks, at least about 60 to 65 weeks, at least about 65 to 70 weeks, at least about 70 to 80 weeks, at least about 80 to 90 weeks, or at least about 90 to 100 in a tumor re-challenge relative to the tumor growth (tumor volume) in a naive animal or subject.

In any of these embodiments in which immunological memory is observed, timing of tumor growth as measured in tumor volume delayed by at least about 0 to 2 years, at least about 2 to 4 years, at least about 4 to 6 years, at least about 6 to 8 years, at least about 8 to 10 years, at least about 10 to 12 years, at least about 12 to 14 years, at least about 14 to 16 years, at least about 16 to 18 years, at least about 18 to 20 years, at least about 20 to 25 years, at least about 25 to 30 years, at least about 30 to 35 years, at least about 35 to 40 years, at least about 40 to 45 years, at least about 45 to 50 years, at least about 50 to 55 years, at least about 55 to 60 years, at least about 60 to 65 years, at least about 65 to 70 years, at least about 70 to 80 years, at least about 80 to 90 years, or at least about 90 to 100 in a tumor re-challenge relative to the tumor growth (tumor volume) in a naive animal or subject.

In yet another embodiment, survival rate is at least about 1.0-1.2-fold, at least about 1.2-1.4-fold, at least about 1.4-1.6-fold, at least about 1.6-1.8-fold, at least about 1.8-2-fold, or at least about two-fold greater in a tumor re-challenge as compared to the tumor growth (tumor volume) in a naive subject. In yet another embodiment, survival rate is at least about 2 to 3-fold, at least about 3 to 4-fold, at least about 4 to 5-fold, at least about 5 to 6-fold, at least about 6 to 7-fold, at least about 7 to 8-fold, at least about 8 to 9-fold, at least about 9 to 10-fold, at least about 10 to 15-fold, at least about 15 to 20-fold, at least about 20 to 30-fold, at least about 30 to 40-fold, or at least about 40 to 50-fold, at least about 50 to 100-fold, at least about 100 to 500-hundred-fold, or at least about 500 to 1000-fold greater in a tumor re-challenge as compared to the tumor growth (tumor volume) in a naive subject.

As used herein, “hot tumors” refer to tumors, which are T cell inflamed, i.e., associated with a high abundance of T cells infiltrating into the tumor. “Cold tumors” are characterized by the absence of effector T cells infiltrating the tumor and are further grouped into “immune excluded” tumors, in which immune cells are attracted to the tumor but cannot infiltrate the tumor microenvironment, and “immune ignored” phenotypes, in which no recruitment of immune cells occurs at all (further reviewed in Van der Woude et al., Migrating into the Tumor: a Roadmap for T Cells. Trends Cancer. 2017 November; 3(11):797-808).

“Hypoxia” is used to refer to reduced oxygen supply to a tissue as compared to physiological levels, thereby creating an oxygen-deficient environment. “Normoxia” refers to a physiological level of oxygen supply to a tissue. Hypoxia is a hallmark of solid tumors and characterized by regions of low oxygen and necrosis due to insufficient perfusion (Groot et al., 2007).

As used herein, the term “low oxygen” is meant to refer to a level, amount, or concentration of oxygen (O₂) that is lower than the level, amount, or concentration of oxygen that is present in the atmosphere (e.g., <21% O₂; <160 torr O₂)). Thus, the term “low oxygen condition or conditions” or “low oxygen environment” refers to conditions or environments containing lower levels of oxygen than are present in the atmosphere.

In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg 02, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties.

In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nonaerobic, microoxic, hypoxic, anoxic, and/or anaerobic conditions. For example, Table 1 summarizes the amount of oxygen present in various organs and tissues. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>.

In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated.

In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.).

The exemplary fractions and ranges listed here are for illustrative purposes and not meant to be limiting in any way. In some embodiments, the term “low oxygen” is meant to refer to 9% O₂ saturation or less, e.g., 9%, 8%, 7%, 6%, 5%, 4%, 3%, 2%, 1%, 0%, O₂ saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of O₂ saturation levels between 0-9%, inclusive (e.g., 0-5%, 0.05-0.1%, 0.1-0.2%, 0.1-0.5%, 0.5-2.0%, 0-8%, 5-7%, 0.3-4.2% O₂, etc.). The exemplary fractions and ranges listed here are for illustrative purposes and not meant to be limiting in any way.

TABLE 1

| Compartment | Oxygen Tension |
|---|---|
| stomach | ~60 torr (e.g., 58 +/− 15 torr) |
| duodenum and first part | ~30 torr (e.g., 32 +/− 8 torr); ~20% |
| of jejunum | oxygen in ambient air |
| Ileum (mid- small | ~10 torr; ~6% oxygen in ambient air |
| intestine) | (e.g., 11 +/− 3 torr) |
| Distal sigmoid colon | ~3 torr (e.g., 3 +/− 1 torr) |
| colon | <2 torr |
| Lumen of cecum | <1 torr |
| tumor | <32 torr (most tumors are <15 torr) |

As used herein, the term “gene” or “gene sequence” refers to any sequence expressing a polypeptide or protein, including genomic sequences, cDNA sequences, naturally occurring sequences, artificial sequences, and codon optimized sequences. The term “gene” or “gene sequence” inter alia includes modification of endogenous genes, such as deletions, mutations, and expression of native and non-native genes under the control of a promoter that that they are not normally associated with in nature.

As used herein the terms “gene cassette” and “circuit” or “circuitry” inter alia refers to any sequence expressing a polypeptide or protein, including genomic sequences, cDNA sequences, naturally occurring sequences, artificial sequences, and codon optimized sequences includes modification of endogenous genes, such as deletions, mutations, and expression of native and non-native genes under the control of a promoter that that they are not normally associated with in nature.

An antibody generally refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer composed of two identical pairs of polypeptide chains, each pair having one “light” (about 25 kD) and one “heavy” chain (about 50-70 kD), connected through a disulfide bond.

As used herein, the term “antibody” or “antibodies” is meant to encompasses all variations of antibody and fragments thereof that possess one or more particular binding specificities. Thus, the term “antibody” or “antibodies” is meant to include full length antibodies, chimeric antibodies, humanized antibodies, single chain antibodies (ScFv, camelids), Fab, Fab′, multimeric versions of these fragments (e.g., F(ab′)2), single domain antibodies (sdAB, V_{H}H fragments), heavy chain antibodies (HCAb), nanobodies, diabodies, and minibodies. Antibodies can have more than one binding specificity, e.g. be bispecific. The term “antibody” is also meant to include so-called antibody mimetics, i.e., which can specifically bind antigens but do not have an antibody-related structure.

A “single-chain antibody” or “single-chain antibodies” typically refers to a peptide comprising a heavy chain of an immunoglobulin, a light chain of an immunoglobulin, and optionally a linker or bond, such as a disulfide bond. The single-chain antibody lacks the constant Fc region found in traditional antibodies. In some embodiments, the single-chain antibody is a naturally occurring single-chain antibody, e.g., a camelid antibody. In some embodiments, the single-chain antibody is a synthetic, engineered, or modified single-chain antibody. In some embodiments, the single-chain antibody is capable of retaining substantially the same antigen specificity as compared to the original immunoglobulin despite the addition of a linker and the removal of the constant regions. In some aspects, the single chain antibody can be a “scFv antibody”, which refers to a fusion protein of the variable regions of the heavy (VH) and light chains (VL) of immunoglobulins (without any constant regions), optionally connected with a short linker peptide of ten to about 25 amino acids, as described, for example, in U.S. Pat. No. 4,946,778, the contents of which is herein incorporated by reference in its entirety. The Fv fragment is the smallest fragment that holds a binding site of an antibody, which binding site may, in some aspects, maintain the specificity of the original antibody. Techniques for the production of single chain antibodies are described in U.S. Pat. No. 4,946,778.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: Ala, Pro, Gly, Gln, Asn, Ser, Thr, Cys, Ser, Tyr, Thr, Val, Ile, Leu, Met, Ala, Phe, Lys, Arg, His, Phe, Tyr, Trp, His, Asp, and Glu.

In any of these combination embodiments, an immune modulator may be fused to a stabilizing polypeptide. Such stabilizing polypeptides are known in the art and include Fc proteins. In some embodiments, the fusion proteins are Fc fusion proteins, such as IgG Fc fusion proteins or IgA Fc fusion proteins.

In some embodiments, an immune modulator, is covalently fused to the stabilizing polypeptide through a peptide linker or a peptide bond. In some embodiments, the stabilizing polypeptide comprises an immunoglobulin Fc polypeptide. In some embodiments, the immunoglobulin Fc polypeptide comprises at least a portion of an immunoglobulin heavy chain CH2 constant region. In some embodiments, the immunoglobulin Fc polypeptide comprises at least a portion of an immunoglobulin heavy chain CH3 constant region. In some embodiments, the immunoglobulin Fc polypeptide comprises at least a portion of an immunoglobulin heavy chain CH1 constant region. In some embodiments, the immunoglobulin Fc polypeptide comprises at least a portion of an immunoglobulin variable hinge region. In some embodiments, the immunoglobulin Fc polypeptide comprises at least a portion of an immunoglobulin variable hinge region, immunoglobulin heavy chain CH2 constant region and an immunoglobulin heavy chain CH3 constant region. In some embodiments, the immunoglobulin Fc polypeptide is a human IgG4 Fc polypeptide. In some embodiments, the linker comprises a glycine rich peptide. In some embodiments, the glycine rich peptide comprises the sequence [GlyGlyGlyGlySer]n where n is 1, 2, 3, 4, 5 or 6 (SEQ ID NO: 1245). In some embodiments, the fusion protein comprises a SIRPα IgG FC fusion polypeptide. In some embodiments, the fusion protein comprises a SIRPα IgG4 Fc polypeptide. In some embodiments, the glycine rich peptide linker comprises the sequence SGGGGSGGGGSGGGGS (SEQ ID NO: 1121). In some embodiments, the N terminus of SIRPα is covalently fused to the C terminus of a IgG4 Fc through the peptide linker comprising SGGGGSGGGGSGGGGS (SEQ ID NO: 1121).

In some embodiments, the immune modulator is a multimeric polypeptide. In some embodiments, the polypeptide is a dimer. Non-limiting example of a dimeric proteins include cytokines, such as IL-15 (heterodimer). In some embodiments, the immune modulator comprises one or more polypeptides wherein the one or more polypeptides comprise a first monomer and a second monomer. In some embodiments, the first monomer polypeptide is covalently linked to a second monomer polypeptide through a peptide linker or a peptide bond. In some embodiments, the linker comprises a glycine rich peptide. In some embodiments, the first and the second monomer have the same polypeptide sequence. In some embodiments, the first and the second monomer have each have a different polypeptide sequence. In some embodiments, the first monomer is a IL-12 p35 polypeptide and the second monomer is a IL-12 p40 polypeptide. In some embodiments, the linker comprises GGGGSGGGS (SEQ ID NO: 1244).

In some embodiments, the immune modulator is a hIGg4 fusion protein which comprises a hIgG4 portion that has about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 1117. In another embodiment, the hIgG4 portion comprises SEQ ID NO: 1117. In yet another embodiment, the hIgG4 portion of the polypeptide consists of SEQ ID NO: 1117.

In some embodiments, the fusion protein comprises a linker portion that has about 80%, 81%, 82%, 83%, 84%, 85%, 86%, 87%, 88%, 89%, 90%, 91%, 92%, 93%, 94%, 95%, 96%, 97%, 98%, or 99% identity with one or more of SEQ ID NO: 1121. In another embodiment, the linker portion comprises SEQ ID NO: 1121. In yet another embodiment, the linker portion of the polypeptide consists of SEQ ID NO: 1121.

In some embodiments, effector function of an immune modulator can be improved through fusion to another polypeptide that facilitates effector function. A non-limiting example of such a fusion is the fusion of IL-15 to the Sushi domain of IL-15Ralpha, as described herein. In some embodiments, accordingly, a first monomer polypeptide is a IL-15 monomer and the second monomer is a IL-15R alpha sushi domain polypeptide.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wild-type peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety. In some embodiments, the linker is a glycine rich linker. In some embodiments, the linker is (Gly-Gly-Gly-Gly-Ser)n. In some embodiments, the linker comprises SEQ ID NO: 979.

The immune system is typically most broadly divided into two categories—innate immunity and adaptive immunity—although the immune responses associated with these immunities are not mutually exclusive. “Innate immunity” refers to non-specific defense mechanisms that are activated immediately or within hours of a foreign agent's or antigen's appearance in the body. These mechanisms include physical barriers such as skin, chemicals in the blood, and immune system cells, such as dendritic cells (DCs), leukocytes, phagocytes, macrophages, neutrophils, and natural killer cells (NKs), that attack foreign agents or cells in the body and alter the rest of the immune system to the presence of the foreign agents. During an innate immune response, cytokines and chemokines are produced which in combination with the presentation of immunological antigens, work to activate adaptive immune cells and initiate a full blown immunologic response. “Adaptive immunity” or “acquired immunity” refers to antigen-specific immune response. The antigen must first be processed or presented by antigen presenting cells (APCs). An antigen-presenting cell or accessory cell is a cell that displays antigens directly or complexed with major histocompatibility complexes (MHCs) on their surfaces. Professional antigen-presenting cells, including macrophages, B cells, and dendritic cells, specialize in presenting foreign antigen to T helper cells in a MHC-II restricted manner, while other cell types can present antigen originating inside the cell to cytotoxic T cells in a MHC-I restricted manner. Once an antigen has been presented and recognized, the adaptive immune system activates an army of immune cells specifically designed to attack that antigen. Like the innate system, the adaptive system includes both humoral immunity components (B lymphocyte cells) and cell-mediated immunity (T lymphocyte cells) components. B cells are activated to secrete antibodies, which travel through the bloodstream and bind to the foreign antigen. Helper T cells (regulatory T cells, CD4+ cells) and cytotoxic T cells (CTL, CD8+ cells) are activated when their T cell receptor interacts with an antigen-bound MHC molecule. Cytokines and co-stimulatory molecules help the T cells mature, which mature cells, in turn, produce cytokines which allows the production of priming and expansion of additional T cells sustaining the response. Once activated, the helper T cells release cytokines which regulate and direct the activity of different immune cell types, including APCs, macrophages, neutrophils, and other lymphocytes, to kill and remove targeted cells. Helper T cells also secrete extra signals that assist in the activation of cytotoxic T cells which also help to sustain the immune response. Upon activation, CTL undergoes clonal selection, in which it gains functions, divides rapidly to produce an army of activated effector cells, and forms long-lived memory T cells ready to rapidly respond to future threats. Activated CTL then travels throughout the body searching for cells that bear that unique MHC Class I and antigen. The effector CTLs release cytotoxins that form pores in the target cell's plasma membrane, causing apoptosis. Adaptive immunity also includes a “memory” that makes future responses against a specific antigen more efficient. Upon resolution of the infection, T helper cells and cytotoxic T cells die and are cleared away by phagocytes, however, a few of these cells remain as memory cells. If the same antigen is encountered at a later time, these memory cells quickly differentiate into effector cells, shortening the time required to mount an effective response.

An “immune checkpoint inhibitor” or “immune checkpoint” refers to a molecule that completely or partially reduces, inhibits, interferes with, or modulates one or more immune checkpoint proteins. Immune checkpoint proteins regulate T-cell activation or function, and are known in the art. Non-limiting examples include CTLA-4 and its ligands CD 80 and CD86, and PD-1 and its ligands PD-L1 and PD-L2. Immune checkpoint proteins are responsible for co-stimulatory or inhibitory interactions of T-cell responses, and regulate and maintain self-tolerance and physiological immune responses.

A “co-stimulatory” molecule or “co-stimulator” is an immune modulator that increases or activates a signal that stimulates an immune response or inflammatory response.

As used herein, an immune modulator that “inhibits” cancerous cells refers to a molecule that is capable of reducing cell proliferation, reducing tumor growth, and/or reducing tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to control, e.g., an untreated control.

As used herein, an immune modulator that “activates” or “stimulates” a biological molecule, e.g., cytokine, chemokine, immune modulatory metabolite, or any other immune modulatory agent, factor, or molecule, refers to an immune modulator that is capable of activating, increasing, enhancing, or promoting the biological activity, biological function, and/or number of that biological molecule, as compared to control, e.g., an untreated control under the same conditions.

“Bacteria for intratumoral administration” refer to bacteria that are capable of directing themselves to cancerous cells. Bacteria for intratumoral administration may be naturally capable of directing themselves to cancerous cells, necrotic tissues, and/or hypoxic tissues.

In some embodiments, bacteria that are not naturally capable of directing themselves to cancerous cells, necrotic tissues, and/or hypoxic tissues are genetically engineered to direct themselves to cancerous cells, necrotic tissues, and/or hypoxic tissues. Bacteria for intratumoral administration may be further engineered to enhance or improve desired biological properties, mitigate systemic toxicity, and/or ensure clinical safety. These species, strains, and/or subtypes may be attenuated, e.g., deleted for a toxin gene.

In some embodiments, bacteria for intratumoral administration have low infection capabilities. In some embodiments, bacteria for intratumoral administration are motile. In some embodiments, the bacteria for intratumoral administration are capable of penetrating deeply into the tumor, where standard treatments do not reach. In some embodiments, bacteria for intratumoral administration are capable of colonizing at least 20%, at least 30%, at least 40%, at least 50%, at least 60%, at least 70%, at least 80%, at least 90%, or at least 95% of a malignant tumor. Examples of bacteria for intratumoral administration include, but are not limited to, Bifidobacterium, Caulobacter, Clostridium, Escherichia coli, Listeria, Mycobacterium, Salmonella, Streptococcus, and Vibrio, e.g., Bifidobacterium adolescentis, Bifidobacterium bifidum, Bifidobacterium breve UCC2003, Bifidobacterium infantis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera (Cronin et al., 2012; Forbes, 2006; Jain and Forbes, 2001; Liu et al., 2014; Morrissey et al., 2010; Nuno et al., 2013; Patyar et al., 2010; Cronin, et al., Mol Ther 2010; 18:1397-407). In some embodiments, the bacteria for intratumoral administration are non-pathogenic bacteria. In some embodiments, intratumoral administration is done via injection.

“Microorganism” refers to an organism or microbe of microscopic, submicroscopic, or ultramicroscopic size that typically consists of a single cell. Examples of microorganisms include bacteria, viruses, parasites, fungi, certain algae, protozoa, and yeast. In some aspects, the microorganism is modified (“modified microorganism”) from its native state. In certain embodiments, the modified microorganism is a modified bacterium. In some embodiments, the modified microorganism is a genetically engineered bacterium. In certain embodiments, the modified microorganism is a modified yeast. In other embodiments, the modified microorganism is a genetically engineered yeast.

As used herein, the term “recombinant microorganism” refers to a microorganism, e.g., bacterial, yeast, or viral cell, or bacteria, yeast, or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial, yeast, or viral cell, or bacteria, yeast, or virus, that has been genetically modified from its native state to perform a specific function. Thus, a “programmed or bacterial cell” or “programmed or bacteria” refers to a bacterial cell or bacteria that has been genetically modified from its native state to perform a specific function. In certain embodiments, the programmed or bacterial cell has been modified to express one or more proteins, for example, one or more proteins that have a therapeutic activity or serve a therapeutic purpose. The programmed or bacterial cell may additionally have the ability to stop growing or to destroy itself once the protein(s) of interest have been expressed.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976).

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to certain strains belonging to the genus Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006).

“Operably linked” refers a nucleic acid sequence that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region. In one embodiment, an inducible promoter is a salicylate promoter. In another embodiment, an inducible promoter is a cumate promoter. In another embodiment, an inducible promoter is a fumarate-and-nitrase reductase promoter.

“Exogenous environmental condition(s)” refer to setting(s) or circumstance(s) under which the promoter described herein is induced. In some embodiments, the exogenous environmental conditions are specific to a malignant growth containing cancerous cells, e.g., a tumor. The phrase “exogenous environmental conditions” is meant to refer to the environmental conditions external to the intact (unlysed) engineered microorganism, but endogenous or native to tumor environment or the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are low-oxygen, microaerobic, or anaerobic conditions, such as hypoxic and/or necrotic tissues. Some solid tumors are associated with low intracellular and/or extracellular pH; in some embodiments, the exogenous environmental condition is a low-pH environment. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 2.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

Accordingly, in one embodiment, the Pfnrs promoter has at least about 85%, 86%, 87%, 88%, 89%, 90%, 91%, 92%, 93%, 94%, 95%, 96%, 97%, 98%, or 99% identity with the sequence of SEQ ID NO: 856. In another embodiment, the Pfnrs promoter comprises the sequence of SEQ ID NO: 856. In yet another embodiment the Pfnrs promoter consists of the sequence of SEQ ID NO: 856.

TABLE 2
Examples of transcription factors and
responsive genes and regulatory regions

| Transcription | Examples of responsive genes, promoters, and/or regulatory |
|---|---|
| Factor | regions: |
| FNR | nirB, ydfZ, pdhR, focA, ndH, hlyE, narK, narX, |
| | narG, yfiD, tdcD |
| ANR | arcDABC |
| DNR | norb, norC |

“Constitutive promoter” refers to a promoter that is capable of facilitating continuous transcription of a coding sequence or gene under its control and/or to which it is operably linked. Constitutive promoters and variants are well known in the art and non-limiting examples of constitutive promoters are described herein and in International Patent Application PCT/US2017/013072, filed Jan. 11, 2017 and published as WO2017/123675, the contents of which is herein incorporated by reference in its entirety. In some embodiments, such promoters are active in vitro, e.g., under culture, expansion and/or manufacture conditions. In some embodiments, such promoters are active in vivo, e.g., in conditions found in the in vivo environment, e.g., the gut and/or the tumor microenvironment.

As used herein, “stably maintained” or “stable” bacterium or virus is used to refer to a bacterial or viral host cell carrying non-native genetic material, such that the non-native genetic material is retained, expressed, and propagated. The stable bacterium or virus is capable of survival and/or growth in vitro, e.g., in medium, and/or in vivo, e.g., in hypoxic and/or necrotic tissues. For example, the stable bacterium or virus may be a genetically engineered bacterium comprising non-native genetic material, in which the plasmid or chromosome carrying the non-native genetic material is stably maintained in the bacterium or virus, such that the protein encoded by the non-native genetic material can be expressed in the bacterium or virus, and the bacterium or virus is capable of survival and/or growth in vitro and/or in vivo.

As used herein, the terms “modulate” and “treat” and their cognates refer to an amelioration of a cancer, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a cancer, either physically (e.g., stabilization of a discernible symptom), physiologically (e.g., stabilization of a physical parameter), or both. In another embodiment, “modulate” and “treat” refer to slowing the progression or reversing the progression of a cancer. As used herein, “prevent” and its cognates refer to delaying the onset or reducing the risk of acquiring a given cancer.

Those in need of treatment may include individuals already having a particular cancer, as well as those at risk of having, or who may ultimately acquire the cancer. The need for treatment is assessed, for example, by the presence of one or more risk factors associated with the development of a cancer (e.g., alcohol use, tobacco use, obesity, excessive exposure to ultraviolet radiation, high levels of estrogen, family history, genetic susceptibility), the presence or progression of a cancer, or likely receptiveness to treatment of a subject having the cancer. Cancer is caused by genomic instability and high mutation rates within affected cells. Treating cancer may encompass eliminating symptoms associated with the cancer and/or modulating the growth and/or volume of a subject's tumor, and does not necessarily encompass the elimination of the underlying cause of the cancer, e.g., an underlying genetic predisposition.

As used herein, the term “conventional cancer treatment” or “conventional cancer therapy” refers to treatment or therapy that is widely accepted and used by most healthcare professionals. It is different from alternative or complementary therapies, which are not as widely used. Examples of conventional treatment for cancer include surgery, chemotherapy, targeted therapies, radiation therapy, tomotherapy, immunotherapy, cancer vaccines, hormone therapy, hyperthermia, stem cell transplant (peripheral blood, bone marrow, and cord blood transplants), photodynamic therapy, therapy, and blood product donation and transfusion.

As used herein a “pharmaceutical composition” refers to a preparation of at least one microorganism of the disclosure and/or at least one immune modulator, with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., a cancer. A therapeutically effective amount may, for example, be sufficient to treat, prevent, reduce the severity, delay the onset, and/or reduce the risk of occurrence of one or more symptoms of a disorder associated with cancerous cells. A therapeutically effective amount, as well as a therapeutically effective frequency of administration, can be determined by methods known in the art and discussed below.

In some embodiments, the term “therapeutic molecule” refers to a molecule or a compound that is results in prevention, delay of onset of symptoms, or amelioration of symptoms of a condition, e.g., a cancer. In some embodiments, a therapeutic molecule may be, for example, a cytokine, a chemokine, a single chain antibody, a ligand, a metabolic converter, e.g., arginine, a kynurenine consumer, or an adenosine consumer, a T cell co-stimulatory receptor, a T cell co-stimulatory receptor ligand, an engineered chemotherapy, or a lytic peptide, among others.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Bacteria

In one embodiment, the microorganism may be a bacterium. The bacteria may be administered systemically, orally, locally and/or intratumorally. In some embodiments, the bacteria are capable of targeting cancerous cells, particularly in the hypoxic regions of a tumor, and are administered in combination with, e.g., an immune modulator, e.g., immune stimulator or sustainer provided herein.

In some embodiments, the tumor-targeting microorganism is a bacterium that is naturally capable of directing itself to cancerous cells, necrotic tissues, and/or hypoxic tissues. For example, bacterial colonization of tumors may be achieved without any specific genetic modifications in the bacteria or in the host (Yu et al., 2008). In some embodiments, the tumor-targeting bacterium is a bacterium that is not naturally capable of directing itself to cancerous cells, necrotic tissues, and/or hypoxic tissues, but is genetically engineered to do so. In some embodiments, the bacteria spread hematogenously to reach the targeted tumor(s). Bacterial infection has been linked to tumor regression (Hall, 1998; Nauts and McLaren, 1990), and certain bacterial species have been shown to localize to and lyse necrotic mammalian tumors (Jain and Forbes, 2001). Non-limiting examples of tumor-targeting bacteria are shown in Table 3.

TABLE 3
Bacteria with tumor-targeting capability

| Bacterial Strain | See, e.g., |
|---|---|
| Clostridium novyi-NT | Forbes, Neil S. “Profile of a bacterial |
| | tumor killer.” Nature biotechnology |
| | 24.12 (2006): 1484-1485. |
| Bifidobacterium spp | Liu, Sai, et al. “Tumor-targeting bacterial |
| Streptococcus spp | therapy: A potential treatment for |
| Caulobacter spp | oral cancer.” Oncology letters |
| Clostridium spp | 8.6 (2014): 2359-2366. |
| Escherichia coli MG1655 | Cronin, Michelle, et al. “High resolution |
| Escherichia coli Nissle | in vivo bioluminescent imaging for the |
| Bifidobacterium breve | study of bacterial tumour targeting.” |
| UCC2003 | PloS one 7.1 (2012): e30940.; |
| Salmonella typhimurium | Zhou, et al., Med Hypotheses. 2011 April; |
| | 76(4): 533-4. doi: |
| | 10.1016/j.mehy.2010.12.010. |
| | Epub 2011 January 21; Zhang et al., Appl |
| | Environ Microbiol. 2012 November; |
| | 78(21): 7603-7610; Danino et al., Science |
| | Translational Medicine, 2015 Vol 7 |
| | Issue 289, pp. 289ra84 |
| Clostridium novyi-NT | Bernardes, Nuno, Ananda M. |
| Bifidobacterium spp | Chakrabarty, and Arsenio M. Fialho. |
| Mycobacterium bovis | “Engineering of bacterial strains and |
| Listeria monocytogenes | their products for cancer therapy.” |
| Escherichia coli | Applied microbiology and biotechnology |
| Salmonella spp | 97.12 (2013): 5189-5199. |
| Salmonella typhimurium | |
| Salmonella choleraesuis | Patyar, S., et al. “Bacteria in cancer |
| Vibrio cholera | therapy: a novel experimental strategy.” |
| Listeria monocytogenes | J Biomed Sci 17.1 (2010): 21-30. |
| Escherichia coli | |
| Bifidobacterium adolescentis | |
| Clostridium acetobutylicum | |
| Salmonella typhimurium | |
| Clostridium histolyticum | |
| Escherichia coli Nissle 1917 | Danino et al. “Programmable probiotics |
| | for detection of cancer in urine.” Sci |
| | Transl Med. 2015 May 27; 7(289): |
| | 289ra84 |

In some embodiments, the bacterium which enhances the efficacy of immunotherapy. Recent studies have suggested that the presence of certain types of gut microbes in mice can enhance the anti-tumor effects of cancer immunotherapy without increasing toxic side effects (M. Vétizou et al., “Anticancer immunotherapy by CTLA-4 blockade relies on the gut microbiota,” Science, doi:10.1126/aad1329, 2015; A. Sivan et al., “Commensal Bifidobacterium promotes antitumor immunity and facilitates anti-PD-L1 efficacy,” Science, doi:0.1126/science.aac4255, 2015). Whether the gut microbial species identified in these mouse studies will have the same effect in humans is not clear. Vetizou et al (2015) describe T cell responses specific for Bacteroides thetaiotaomicron or Bacteroides fragilis that were associated with the efficacy of CTLA-4 blockade in mice and in patients. Sivan et al. (2015) illustrate the importance of Bifidobacterium to antitumor immunity and anti-PD-L1 antibody against (PD-1 ligand) efficacy in a mouse model of melanoma.

In some embodiments, the bacteria are Bacteroides. In some embodiments, the bacteria are Bifidobacterium. In some embodiments, the bacteria are Escherichia Coli Nissle. In some embodiments, the bacteria are Clostridium novyi-NT. In some embodiments, the bacteria are Clostridium butyricum miyairi.

In certain embodiments, the microorganisms are obligate anaerobic bacteria. In certain embodiments, the bacteria are facultative anaerobic bacteria. In certain embodiments, the bacteria are aerobic bacteria. In some embodiments, the bacteria are Gram-positive bacteria and lack LPS. In some embodiments, the bacteria are Gram-negative bacteria. In some embodiments, the bacteria are Gram-positive and obligate anaerobic bacteria. In some embodiments, the bacteria are Gram-positive and facultative anaerobic bacteria. In some embodiments, the bacteria are non-pathogenic bacteria. In some embodiments, the bacteria are commensal bacteria. In some embodiments, the bacteria are probiotic bacteria.

Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, Vibrio cholera, and the bacteria shown in Table 3. In certain embodiments, the bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis.

In some embodiments, Lactobacillus is used. Lactobacillus casei injected intravenously has been found to accumulate in tumors, which was enhanced through nitroglycerin (NG), a commonly used NO donor, likely due to the role of NO in increasing the blood flow to hypovascular tumors (Fang et al., 2016 (Methods Mol Biol. 2016; 1409:9-23. Enhancement of Tumor-Targeted Delivery of Bacteria with Nitroglycerin Involving Augmentation of the EPR Effect).

In some embodiments, the bacteria are obligate anaerobes. In some embodiments, the bacteria are Clostridia. Clostridia are obligate anaerobic bacterium that produce spores and are naturally capable of colonizing and in some cases lysing hypoxic tumors (Groot et al., 2007). In experimental models, Clostridia have been used to deliver pro-drug converting enzymes and enhance radiotherapy (Groot et al., 2007). In some embodiments, the bacteria is selected from the group consisting of Clostridium novyi-NT, Clostridium histolyticium, Clostridium tetani, Clostridium oncolyticum, Clostridium sporogenes, and Clostridium beijerinckii (Liu et al., 2014). In some embodiments, the Clostridium is naturally non-pathogenic. For example, Clostridium oncolyticum is a pathogenic and capable of lysing tumor cells. In alternate embodiments, the Clostridium is naturally pathogenic but modified to reduce or eliminate pathogenicity. For example, Clostridium novyi are naturally pathogenic, and Clostridium novyi-NT are modified to remove lethal toxins. Clostridium novyi-NT and Clostridium sporogenes have been used to deliver single-chain HIF-1a antibodies to treat cancer and is an “excellent tumor colonizing Clostridium strains” (Groot et al., 2007).

In some embodiments, the bacteria facultative anaerobes. In some embodiments, the bacteria are Salmonella, e.g., Salmonella typhimurium. Salmonella are non-spore-forming Gram-negative bacteria that are facultative anaerobes. In some embodiments, the Salmonella are naturally pathogenic but modified to reduce or eliminate pathogenicity. For example, Salmonella typhimurium is modified to remove pathogenic sites (attenuated). In some embodiments, the bacteria are Bifidobacterium. Bifidobacterium are Gram-positive, branched anaerobic bacteria. In some embodiments, the Bifidobacterium is naturally non-pathogenic. In alternate embodiments, the Bifidobacterium is naturally pathogenic but modified to reduce or eliminate pathogenicity. Bifidobacterium and Salmonella have been shown to preferentially target and replicate in the hypoxic and necrotic regions of tumors (Yu et al., 2014).

In some embodiments, the bacteria are Gram-negative bacteria. In some embodiments, the bacteria are E. coli. For example, E. coli Nissle has been shown to preferentially colonize tumor tissue in vivo following either oral or intravenous administration (Zhang et al., 2012 and Danino et al., 2015). E. coli have also been shown to exhibit robust tumor-specific replication (Yu et al., 2008). In some embodiments, the bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added).

In some embodiments, the bacteria are administered repeatedly. In some embodiments, the bacteria are administered once.

Further examples of bacteria which are suitable are described in International Patent Publication WO/2014/043593, the contents of which are herein incorporated by reference in its entirety. In some embodiments, such bacteria are mutated to attenuate one or more virulence factors. Other bacteria are described at least in Song et al., Infectious Agents and Cancer, 2018; and Lukasiewicz and Fol, J. Immunol. Research, 2018, Article ID 2397808.

In some embodiments, the bacteria of the disclosure proliferate and colonize a tumor. In some embodiments, colonization persists for several days, several weeks, several months, several years or indefinitely. In some embodiments, the bacteria do not proliferate in the tumor and bacterial counts drop off quickly post injection, e.g., less than a week post injection, until no longer detectable.

### Essential Genes and Auxotrophs

As used herein, the term “essential gene” refers to a gene that is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the bacteria described herein also comprise a deletion or mutation in a gene required for cell survival and/or growth. In one embodiment, the essential gene is a DNA synthesis gene, for example, thyA. In another embodiment, the essential gene is a bacterial cell wall synthesis gene, for example, dapA. In yet another embodiment, the essential gene is an amino acid gene, for example, serA or metA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria. Exemplary bacterial genes which may be disrupted or deleted to produce an auxotrophic strain as described in International Patent Application PCT/US2017/013072, filed Jan. 11, 2017, published as WO2017/123675, the contents of which is herein incorporated by reference in its entirety. These include, but are not limited to, genes required for oligonucleotide synthesis, amino acid synthesis, and cell wall synthesis. Table 4 lists exemplary bacterial genes which may be disrupted or deleted to produce an auxotrophic strain. These include, but are not limited to, genes required for oligonucleotide synthesis, amino acid synthesis, and cell wall synthesis.

TABLE 4
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Auxotrophic mutations are useful in some instances in which biocontainment strategies may be required to prevent unintended proliferation of the bacterium in a natural ecosystem. Any auxotrophic mutation in an essential gene described above or known in the art can be useful for this purpose, e.g. DNA synthesis genes, amino acid synthesis genes, or genes for the synthesis of cell wall. Accordingly, in some embodiments, the bacteria comprise modifications, e.g., mutation(s) or deletion(s) in one or more auxotrophic genes, e.g., to prevent growth and proliferation of the bacterium in the natural environment. In some embodiments, the modification may be located in a non-coding region. In some embodiments, the modifications result in attenuation of transcription or translation. In some embodiments, the modifications, e.g., mutations or deletions, result in reduced or no transcription or reduced or no translation of the essential gene. In some embodiments, the modifications, e.g., mutations or deletions, result in transcription and/or translation of a non-functional version of the essential gene. In some embodiments, the modifications, e.g., mutations or deletions result in in truncated transcription or translation of the essential gene, resulting in a truncated polypeptide. In some embodiments, the modification, e.g., mutation is located within the coding region of the gene.

While unable to grow in the natural ecosystem, certain auxotrophic mutations may allow growth and proliferation in the mammalian host administered the bacteria, e.g., in the tumor environment. For example, an essential pathway that is rendered non-functional by the auxotrophic mutation may be complemented by production of the metabolite by the host within the tumor microenvironment. As a result, the bacterium administered to the host can take up the metabolite from the environment and can proliferate and colonize the tumor. Thus, in some embodiments, the auxotrophic gene is an essential gene for the production of a metabolite, which is also produced by the mammalian host in vivo, e.g., in a tumor setting. In some embodiments, metabolite production by the host tumor may allow uptake of the metabolite by the bacterium and permit survival and/or proliferation of the bacterium within the tumor. In some embodiments, bacteria comprising such auxotrophic mutations are capable of proliferating and colonizing the tumor to the same extent as a bacterium of the same subtype which does not carry the auxotrophic mutation.

In some embodiments, the bacteria are capable of colonizing and proliferating in the tumor microenvironment. In some embodiments, the tumor colonizing bacteria comprise one or more auxotrophic mutations. In some embodiments, the tumor colonizing bacteria do not comprise one or more auxotrophic modifications or mutations. In a non-limiting example, greater numbers of bacteria are detected after 24 hours and 72 hours than were originally injected into the subject. In some embodiments, CFUs detected 24 hours post injection are at least about 1 to 2 logs greater than administered. In some embodiments, CFUs detected 24 hours post injection are at least about 2 to 3 logs greater than administered. In some embodiments, CFUs detected 24 hours post injection are at least about 3 to 4 logs greater than administered. In some embodiments, CFUs detected 24 hours post injection are at least about 4 to 5 logs greater than administered. In some embodiments, CFUs detected 24 hours post injection are at least about 5 to 6 logs greater than administered. In some embodiments, CFUs detected 72 hours post injection are at least about 1 to 2 logs greater than administered. In some embodiments, CFUs detected 72 hours post injection are at least about 2 to 3 logs greater than administered. In some embodiments, CFUs detected 72 hours post injection are at least about 3 to 4 logs greater than administered. In some embodiments, CFUs detected 72 hours post injection are at least about 4 to 5 logs greater than administered. In some embodiments, CFUs detected 72 hours post injection are at least about 5 to 6 logs greater than administered. In some embodiments, CFUs can be measured at later time points, such as after at least one week, after at least 2 or more weeks, after at least one month, after at least two or more months post injection.

Non-limiting examples of such auxotrophic genes, which allow proliferation and colonization of the tumor, are thyA and uraA, as shown herein. Accordingly, in some embodiments, the bacteria of the disclosure may comprise an auxotrophic modification, e.g., mutation or deletion, in the thyA gene. In some embodiments, the bacteria of the disclosure may comprise an auxotrophic modification, e.g., mutation or deletion, in the uraA gene. In some embodiments, the bacteria of the disclosure may comprise auxotrophic modification, e.g., mutation or deletion, in the thyA gene and the uraA gene.

Alternatively, the auxotrophic gene is an essential gene for the production of a metabolite which cannot be produced by the host within the tumor, i.e., the auxotrophic mutation is not complemented by production of the metabolite by the host within the tumor microenvironment. As a result, the this mutation may affect the ability of the bacteria to grow and colonize the tumor and bacterial counts decrease over time. This type of auxotrophic mutation can be useful for the modulation of in vivo activity of the immune modulator or duration of activity of the immune modulator, e.g., within a tumor. An example of this method of fine-tuning levels and timing of immune modulator release is described herein using a auxotrophic modification, e.g., mutation, in dapA. Diaminopimelic acid (Dap) is a characteristic component of certain bacterial cell walls, e.g., of gram negative bacteria. Without diaminopimelic acid, bacteria are unable to form proteoglycan, and as such are unable to grow. DapA is not produced by mammalian cells, and therefore no alternate source of DapA is provided in the tumor. As such, a dapA auxotrophy may present a particularly useful strategy to modulate and fine tune timing and extent of bacterial presence in the tumor and/or levels and timing of immune modulator expression and production. Accordingly, in some embodiments, the bacteria of the disclosure comprise an mutation in an essential gene for the production of a metabolite which cannot be produced by the host within the tumor. In some embodiments, the auxotrophic mutation is in a gene which is essential for the production and maintenance of the bacterial cell wall known in the art or described herein, or a mutation in a gene that is essential to another structure that is unique to bacteria and not present in mammalian cells. In some embodiments, bacteria comprising such auxotrophic mutations are capable of proliferating and colonizing the tumor to a substantially lesser extent than a bacterium of the same subtype which does not carry the auxotrophic mutation. Control of bacterial growth (and by extent effector levels) may be further combined with other regulatory strategies, including but not limited to, metabolite or chemically inducible promoters described herein.

In a non-limiting example, lower numbers of bacteria are detected after 24 hours and 72 hours than were originally injected into the subject. In some embodiments, CFUs detected 24 hours post injection are at least about 1 to 2 logs lower than administered. In some embodiments, CFUs detected 24 hours post injection are at least about 2 to 3 logs lower than administered. In some embodiments, CFUs detected 24 hours post injection are at least about 3 to 4 logs lower than administered. In some embodiments, CFUs detected 24 hours post injection are at least about 4 to 5 logs lower than administered. In some embodiments, CFUs detected 24 hours post injection are at least about 5 to 6 logs lower than administered. In some embodiments, CFUs detected 72 hours post injection are at least about 1 to 2 logs lower than administered. In some embodiments, CFUs detected 72 hours post injection are at least about 2 to 3 logs lower than administered. In some embodiments, CFUs detected 72 hours post injection are at least about 3 to 4 logs lower than administered. In some embodiments, CFUs detected 72 hours post injection are at least about 4 to 5 logs lower than administered. In some embodiments, CFUs detected 72 hours post injection are at least about 5 to 6 logs lower than administered. In some embodiments, CFUs can be measured at later time points, such as after at least one week, after at least 2 or more weeks, after at least one month, after at least two or more months post injection.

In some embodiments, the bacteria of the disclosure comprise a auxotrophic modification, e.g., mutation, in dapA. trpE is another auxotrophic mutation described herein. Bacteria carrying this mutation cannot produce tryptophan. In some embodiments, the bacteria comprise auxotrophic mutation(s) in one essential gene. In some embodiments, the bacteria comprise auxotrophic mutation(s) in two essential genes (double auxotrophy). In some embodiments, the bacteria comprise auxotrophic mutation(s) in three or more essential gene(s).

In some embodiments, the bacteria comprise auxotrophic mutation(s) in dapA and thyA. In some embodiments, the bacteria comprise auxotrophic mutation(s) in dapA and uraA. In some embodiments, the bacteria comprise auxotrophic mutation(s) in thyA and uraA. In some embodiments, the bacteria comprise auxotrophic mutation(s) in dapA, thyA and uraA.

In some embodiments, the bacteria comprise auxotrophic mutation(s) in trpE. In some embodiments, the bacteria comprise auxotrophic mutation(s) in trpE and thyA. In some embodiments, the bacteria comprise auxotrophic mutation(s) in trpE and dapA. In some embodiments, the bacteria comprise auxotrophic mutation(s) in trpE and uraA. In some embodiments, the bacteria comprise auxotrophic mutation(s) in trpE, dapA and thyA. In some embodiments, the bacteria comprise auxotrophic mutation(s) in trpE, dapA and uraA. In some embodiments, the bacteria comprise auxotrophic mutation(s) in trpE, thyA and uraA. In some embodiments, the bacteria comprise auxotrophic mutation(s) in trpE, dapA, thyA and uraA.

In another non-limiting example, a conditional auxotroph can be generated. The chromosomal copy of dapA or thyA is knocked out. Another copy of thyA or dapA is introduced, e.g., under control of a low oxygen promoter. Under anaerobic conditions, dapA or thyA—as the case may be—are expressed, and the strain can grow in the absence of dap or thymidine. Under aerobic conditions, dapA or thyA expression is shut off, and the strain cannot grow in the absence of dap or thymidine. Such a strategy can also be employed to allow survival of bacteria under anaerobic conditions, e.g., the gut or conditions of the tumor microenvironment, but prevent survival under aerobic conditions.

In some embodiments, the bacteria comprise auxotrophic mutation(s) in thyA gene, wherein the thyA gene sequence(s) encoding one or more ThyA polypeptide(s) comprising SEQ ID NO: 1004 or functional fragments thereof. In some embodiments, the bacteria comprise auxotrophic mutation(s) in thyA gene, wherein the thyA gene sequence encoding a polypeptide that has at least about 80%, at least about 85%, at least about 90%, at least about 95%, or at least about 99% identity to SEQ ID NO: 1004 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1004. In some specific embodiments, the polypeptide comprises SEQ ID NO: 1004. In other specific embodiments, the polypeptide consists of SEQ ID NO: 1004.

In some embodiments, the bacteria comprise auxotrophic mutation(s) in thyA gene, wherein the thyA gene sequence(s) comprising SEQ ID NO: 1001 or functional fragments thereof. In some embodiments, the bacteria comprise auxotrophic mutation(s) in thyA gene, wherein the thyA gene sequence has at least about 80%, at least about 85%, at least about 90%, at least about 95%, or at least about 99% identity to SEQ ID NO: 1001. In some embodiments, the thyA gene has at least about 85%, 86%, 87%, 88%, 89%, 90%, 91%, 92%, 93%, 94%, 95%, 96%, 97%, 98%, or 99% identity with SEQ ID NO: 1001. In some specific embodiments, the thyA gene comprises SEQ ID NO: 1001. In other specific embodiments, the thyA gene consists of SEQ ID NO: 1001.

In some embodiments, the bacteria comprise auxotrophic mutation(s) in dapA gene, wherein the dapA gene sequence(s) encoding one or more DapA polypeptide(s) comprising SEQ ID NO: 1003 or functional fragments thereof. In some embodiments, the bacteria comprise auxotrophic mutation(s) in dapA gene, wherein the dapA gene sequence encoding a polypeptide that has at least about 80%, at least about 85%, at least about 90%, at least about 95%, or at least about 99% identity to SEQ ID NO: 1003 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1003. In some specific embodiments, the polypeptide comprises SEQ ID NO: 1003. In other specific embodiments, the polypeptide consists of SEQ ID NO: 1003.

In some embodiments, the bacteria comprise auxotrophic mutation(s) in dapA gene, wherein the dapA gene sequence(s) comprising SEQ ID NO: 1000. In some embodiments, the bacteria comprise auxotrophic mutation(s) in dapA gene, wherein the dapA gene sequence has at least about 80%, at least about 85%, at least about 90%, at least about 95%, or at least about 99% identity to SEQ ID NO: 1000. In some embodiments, the dapA gene has at least about 85%, 86%, 87%, 88%, 89%, 90%, 91%, 92%, 93%, 94%, 95%, 96%, 97%, 98%, or 99% identity with SEQ ID NO: 1000. In some specific embodiments, the dapA gene comprises SEQ ID NO: 1000. In other specific embodiments, the dapA gene consists of SEQ ID NO: 1000.

In some embodiments, the bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference). SLiDE bacterial cells are described in International Patent Application PCT/US2017/013072, filed Jan. 11, 2017, published as WO2017/123675, the contents of which is herein incorporated by reference in its entirety.

### Phage Deletion

In some embodiments, the genetically engineered bacteria comprise one or more E. coli Nissle bacteriophage, e.g., Phage 1, Phage 2, and Phage 3. In some embodiments, the genetically engineered bacteria comprise one or mutations in Phage 3. Such mutations include deletions, insertions, substitutions and inversions and are located in or encompass one or more Phage 3 genes. In some embodiments, the one or more insertions comprise an antibiotic cassette. In some embodiments, the mutation is a deletion. In some embodiments, the genetically engineered bacteria comprise one or more deletions, which are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 1447 is deleted from the Phage 3 genome. In one embodiment, a sequence comprising SEQ ID NO: 1447 is deleted from the Phage 3 genome.

In one embodiment, a phage deletion, wherein the phage deletion is encoded by a gene sequence having at least 90%, 91%, 92%, 93%, 94%, 95%, 96%, 97%, 98%, or 99% identity to, comprises, or consists of SEQ ID NO: 1447.

### Activation of Antigen Presenting Cells

### STING Agonists

Stimulator of interferon genes (STING) protein was shown to be a critical mediator of the signaling triggered by cytosolic nucleic acid derived from DNA viruses, bacteria, and tumor-derived DNA. The ability of STING to induce type I interferon production lead to studies in the context of antitumor immune response, and as a result, STING has emerged to be a potentially potent target in anti-tumor immunotherapies. A large part of the antitumor effects caused by STING activation may depend upon production of IFN-0 by APCs and improved antigen presentation by these cells, which promotes CD8+ T cell priming against tumor-associated antigens. However, STING protein is also expressed broadly in a variety of cell types including myeloid-derived suppressor cells (MDSCs) and cancer cells themselves, in which the function of the pathway has not yet been well characterized (Sokolowska, O. & Nowis, D; STING Signaling in Cancer Cells: Important or Not?; Archivum Immunologiae et Therapiae Experimentalis; Arch. Immunol. Ther. Exp. (2018) 66: 125).

Stimulator of interferon genes (STING), also known as transmembrane protein 173 (TMEM173), mediator of interferon regulatory factor 3 activation (MITA), MPYS or endoplasmic reticulum interferon stimulator (ERIS), is a dimeric protein which is mainly expressed in macrophages, T cells, dendritic cells, endothelial cells, and certain fibroblasts and epithelial cells. STING plays an important role in the innate immune response—mice lacking STING are viable though prone to lethal infection following exposure to a variety of microbes. STING functions as a cytosolic receptor for the second messengers in the form of cytosolic cyclic dinucleotides (CDNs), such as cGAMP and the bacterial second messengers c-di-GMP and c-di-AMP. Upon stimulation by the CDN a conformational change in STING occurs. STING translocates from the ER to the Golgi apparatus and its carboxyterminus is liberated, This leads to the activation of TBK1 (TANK-binding kinase 1)/IRF3 (interferon regulatory factor 3), NF-κB, and STAT6 signal transduction pathways, and thereby promoting type I interferon and proinflammatory cytokine responses. CDNs include canonical cyclic di-GMP (c[G(30-50)pG(30-50)p] or cyclic di-AMP or cyclic GAMP (cGMP-AMP) (Barber, STING-dependent cytosolic DNA sensing pathways; Trends Immunol. 2014 February; 35(2):88-93).

CDNs can be exogenously (i.e., bacterially) and/or endogenously produced (i.e., within the host by a host enzyme upon exposure to dsDNA). STING is able to recognize various bacterial second messenger molecules cyclic diguanylate monophosphate (c-di-GMP) and cyclic diadenylate monophosphate (c-di-AMP), which triggers innate immune signaling response (Ma et al., The cGAS-STING Defense Pathway and Its Counteraction by Viruses; Cell Host & Microbe 19, Feb. 10, 2016). Additionally cyclic GMPAMP (cGAMP) can also bind to STING and result inactivation of IRF3 and β-interferon production. Both 3′5′-3′5′ cGAMP (3′3′ cGAMP) produced by Vibrio cholerae, and the metazoan secondary messenger cyclic [G(2′,5′)pA(3′5′)] (2′3′ cGAMP), could activate the innate immune response through STING pathway (Yi et al., Single Nucleotide Polymorphisms of Human STING Can Affect Innate Immune Response to Cyclic Dinucleotides; PLOS One (2013). 8(10)e77846, an references therein). Bacterial and metazoan (e.g., human) c-di-GAMP synthases (cGAS) utilizes GTP and ATP to generate cGAMP capable of STING activation. In contrast to prokaryotic CDNs, which have two canonical 30-50 phosphodiester linkages, the human cGAS product contains a unique 20-50 bond resulting in a mixed linkage cyclic GMP-AMP molecule, denoted as 2′,3′ cGAMP (as described in (Kranzusch et al., Ancient Origin of cGAS-STING Reveals Mechanism of Universal 2′,3′ cGAMP Signaling; Molecular Cell 59, 891-903, Sep. 17, 2015 and references therein). The bacterium Vibrio cholerae encodes an enzyme called DncV that is a structural homolog of cGAS and synthesizes a related second messenger with canonical 3′-5′ bonds (3′,3′ cGAMP).

Components of the stimulator of interferon genes (STING) pathway plays an important role in the detection of tumor cells by the immune system. In preclinical studies, cyclic dinucleotides (CDN), naturally occurring or rationally designed synthetic derivatives, are able to promote an aggressive antitumor response. For example, when co-formulated with an irradiated GM-CSF-secreting whole-cell vaccine in the form of STINGVAX, synthetic CDNs increased the antitumor efficacy and STINGVAX combined with PD-1 blockade induced regression of established tumors (Fu et al., STING agonist formulated cancer vaccines can cure established tumors resistant to PD-1 blockade; Sci Transl Med. 2015 Apr. 15; 7(283): 283ra52). In another example, Smith et al. conducted a study showing that STING agonists may augment CAR T therapy by stimulating the immune response to eliminate tumor cells that are not recognized by the adoptively transferred lymphocytes and thereby improve the effectiveness of CAR T cell therapy (Smith et al., Biopolymers co-delivering engineered T cells and STING agonists can eliminate heterogeneous tumors; J Clin Invest. 2017 Jun. 1; 127(6):2176-2191).

In some embodiments, the recombinant microorganism expresses a STING agonist. Non limiting examples of STING agonists include 3′3′ cGAMP, 2′3′cGAMP, 2′2′-cGAMP, 2′2′-cGAMP VacciGrade™ (Cyclic [G(2′,5′)pA(2′,5′)p]), 2′3′-cGAMP, 2′3′-cGAMP VacciGrade™ (Cyclic [G(2′,5′)pA(3′,5′)p]), 2′3′-cGAM(PS)2 (Rp/Sp), 3′3′-cGAMP, 3′3′-cGAMP VacciGrade™ (Cyclic [G(3′,5′)pA(3′,5′)p]), c-di-AMP, c-di-AMP VacciGrade™ (Cyclic diadenylate monophosphate Th1/Th2 response), 2′3′-c-di-AMP, 2′3′-c-di-AM(PS)2 (Rp,Rp) (Bisphosphorothioate analog of c-di-AMP, Rp isomers), 2′3′-c-di-AM(PS)2 (Rp,Rp) VacciGrade™ c-di-GMP, c-di-GMP VacciGrade™, 2′3′-c-di-GMP, and c-di-IMP.

In one embodiment, the STING agonist is c-diAMP. In one embodiment, the STING agonist is c-GAMP. In one embodiment, the STING agonist is c-diGMP.

In one embodiment, the modified microorganism comprises at least one gene sequence encoding an enzyme which produces the STING agonist. In one embodiment, the at least one gene sequence is a dacA gene sequence. In one embodiment, the dacA gene sequence is a dacA gene sequence from Listeria monocytogenes. In some embodiments, the dacA gene is integrated into the chromosome.

In one embodiment, the at least one gene sequence is a cGAS gene sequence. In one embodiment, the cGAS gene sequence is a human cGAS gene sequence. In one embodiment, the cGAS gene sequence is selected from a human cGAS gene sequence a Verminephrobacter eiseniae cGAS gene sequence, Kingella denitrificans cGAS gene sequence, and a Neisseria bacilliformis cGAS gene sequence.

DacA may be under the control of a constitutive or inducible promoter, e.g., low oxygen inducible promoter such as FNR or any other constitutive or inducible promoter described herein. In some embodiments, the FNR promoter nucleotide sequence has at least about 85%, 86%, 87%, 88%, 89%, 90%, 91%, 92%, 93%, 94%, 95%, 96%, 97%, 98%, or 99% identity to, comprises, or consists of SEQ ID NO: 1282. In some embodiments, the FNR promoter nucleotide sequence is SEQ ID NO: 1282.

In some embodiments, the genetically engineered bacteria comprise dacA gene sequence(s) encoding one or more DacA polypeptide(s) comprising SEQ ID NO: 1209 or functional fragments thereof. In some embodiments, genetically engineered bacteria comprise a gene sequence encoding a polypeptide that has at least about 80%, at least about 85%, at least about 90%, at least about 95%, or at least about 99% identity to SEQ ID NO: 1209 or a functional fragment thereof. In some embodiments, the polypeptide has at least about 85%, 86%, 87%, 88%, 89%, 90%, 91%, 92%, 93%, 94%, 95%, 96%, 97%, 98%, or 99% identity with SEQ ID NO: 1209. In some specific embodiments, the polypeptide comprises SEQ ID NO: 1209. In other specific embodiments, the polypeptide consists of SEQ ID NO: 1209.

(SEQ ID NO: 1209)
MDFSNMSILHYLANIVDILVVWFVIYKVIMLIRGTKAVQLLKGIFIIIAV
KLLSGFFGLQTVEWITDQMLTWGFLAIIIIFQPELRRALETLGRGNIFTR
YGSRIEREQHHLIESIEKSTQYMAKRRIGALISVARDTGMDDYIETGIPL
NAKISSQLLINIFIPNTPLHDGAVIIKGNEIASAASYLPLSDSPFLSKEL
GTRHRAALGISEVTDSITIVVSEETGGISLTKGGELFRDVSEEELHKILL
KELVTVTAKKPSIFSKWKGGKSE

In certain embodiments, the dacA sequence has at least about 80% identity with SEQ ID NO: 1210. In certain embodiments, the dacA gene sequence has at least about 90% identity with SEQ ID NO: 1210. In certain embodiments, the dacA gene sequence has at least about 95% identity with SEQ ID NO: 1210. In some embodiments, the dacA gene sequence has at least about 85%, 86%, 87%, 88%, 89%, 90%, 91%, 92%, 93%, 94%, 95%, 96%, 97%, 98%, or 99% identity with SEQ ID NO: 1210. In some specific embodiments, the dacA gene sequence comprises SEQ ID NO: 1210. In other specific embodiments, the dacA gene sequence consists of SEQ ID NO: 1210.

(SEQ ID NO: 1210)
atggactttt ccaacatgag tatccttcac tatttagcga
atattgtaga cattttggtg gtttggttcg taatttacaa
ggttatcatg cttatccgcg gcacgaaagc cgtccagctg
ttgaagggga ttttcattat tattgccgtc aagttactta
gcggcttctt cggattgcag acagtggaat ggattactga
tcaaatgctg acttggggtt tcttagccat tatcattatc
tttcaaccgg aattgcgtcg tgccctggag actttggggc
gtggcaatat ctttacccgc tatggatcac gcattgagcg
tgaacaacac caccttattg agtctattga aaagtccacg
caatacatgg cgaagcgtcg cattggagct ttgatctctg
tggctcgtga tacaggcatg gacgactaca tcgagactgg
cattccgctt aatgcgaaaa tctcatcgca attattgatt
aatatcttca tccccaatac ccctcttcac gatggcgcag
tgatcattaa aggtaacgag atcgcgagcg ctgccagtta
tctgccattg tccgactcgc cgtttctttc taaggagctg
ggaactcgcc atcgtgccgc attagggatt tccgaggtga
cagattcaat cacaatcgtt gtcagcgagg aaacaggtgg
gatttccctt acgaagggag gcgaactgtt ccgtgatgta
tccgaagaag aattgcataa gattttgctt aaagagctgg
tgactgtcac agctaaaaaa ccaagcattt tctccaaatg
gaaaggaggt aagtccgagt ga

In some embodiments, the genetically engineered bacteria comprise dacA gene sequence(s) operably linked to a promoter which is inducible under low oxygen conditions, e.g., an FNR inducible promoter as described herein. In certain embodiments, the sequence of the dacA gene operably linked to the FNR inducible promoter has at least about 80%, 85%, 90%, 91%, 92%, 93%, 94%, 95%, 96%, 97%, 98%, 99% identity with, comprises, or consists of with SEQ ID NO: 1284. In certain embodiments, the sequence of the dacA gene operably linked to the FNR inducible promoter has at least about 90% identity with SEQ ID NO: 1284. In certain embodiments, the sequence of the dacA gene operably linked to the FNR inducible promoter has at least about 95% identity with SEQ ID NO: 1284. In some embodiments, the sequence of the dacA gene operably linked to the FNR inducible promoter has at least about 85%, 86%, 87%, 88%, 89%, 90%, 91%, 92%, 93%, 94%, 95%, 96%, 97%, 98%, or 99% identity with SEQ ID NO: 1284. In some specific embodiments, the sequence of the dacA gene operably linked to the FNR inducible promoter comprises SEQ ID NO: 1284. In other specific embodiments the sequence of the dacA gene operably linked to the FNR inducible promoter consists of SEQ ID NO: 1284.

(SEQ ID NO: 1284)
agttgttctt attggtggtg ttgctttatg gttgcatcgt
agtaaatggt tgtaacaaaa gcaatttttc cggctgtctg
tatacaaaaa cgccgcaaag tttgagcgaa gtcaataaac
tctctaccca ttcagggcaa tatctctctt ggatccaaag
tgaacccgca tcccacgcta actaatataa ggggggcaag
aatggacttt tccaacatga gtatccttca ctatttagcg
aatattgtag acattttggt ggtttggttc gtaatttaca
aggttatcat gcttatccgc ggcacgaaag ccgtccagct
gttgaagggg attttcatta ttattgccgt caagttactt
agcggcttct tcggattgca gacagtggaa tggattactg
atcaaatgct gacttggggt ttcttagcca ttatcattat
ctttcaaccg gaattgcgtc gtgccctgga gactttgggg
cgtggcaata tctttacccg ctatggatca cgcattgagc
gtgaacaaca ccaccttatt gagtctattg aaaagtccac
gcaatacatg gcgaagcgtc gcattggagc tttgatctct
gtggctcgtg atacaggcat ggacgactac atcgagactg
gcattccgct taatgcgaaa atctcatcgc aattattgat
taatatcttc atccccaata cccctcttca cgatggcgca
gtgatcatta aaggtaacga gatcgcgagc gctgccagtt
atctgccatt gtccgactcg ccgtttcttt ctaaggagct
gggaactcgc catcgtgccg cattagggat ttccgaggtg
acagattcaa tcacaatcgt tgtcagcgag gaaacaggtg
ggatttccct tacgaaggga ggcgaactgt tccgtgatgt
atccgaagaa gaattgcata agattttgct taaagagctg
gtgactgtca cagctaaaaa accaagcatt ttctccaaat
ggaaaggagg taagtccgag tga

### Activation and Priming of Effector Immune Cells (Immune Stimulators)

### Cytokines and Cytokine Receptors

CD4 (4) is a glycoprotein found on the surface of immune cells such as cells, monocytes, macrophages, and dendritic cells. CD4+T helper cells are white blood cells that function to send signals to other types of immune cells, thereby assisting other immune cells in immunologic processes, including maturation of B cells Into plasma cells and memory B cells, and activation of cytotoxic T cells and macrophages. T helper cells become activated when they are presented with peptide antigens by MHC class II molecules, which are expressed on the surface of antigen-presenting cells (APCs). Once activated, T helper cells divide and secrete cytokines that regulate or assist in the active immune response. T helper cells can differentiate into one of several subtypes, including TH1, TH2, TH3, TH17, TH9, or TFH cells, which secrete different cytokines to facilitate different types of immune responses.

Cytotoxic T cells (TC cells, or CTLs) destroy virus-infected cells and tumor cells, and are also implicated in transplant rejection. These cells are also known as CD8+ T cells since they express the CD8 glycoprotein at their surfaces. Cytotoxic T cells recognize their targets by binding to antigen associated with MHC class I molecules, which are present on the surface of all nucleated cells.

In some embodiments, the immune modulator modulates one or more T effector cells, e.g., CD4+ cell and/or CD8+ cell. In some embodiments, the immune modulator that activate, stimulate, and/or induce the differentiation of one or more T effector cells, e.g., CD4+ and/or CD8+ cells. In some embodiments, the immune modulator is a cytokine that activates, stimulates, and/or induces the differentiation of a T effector cell, e.g., CD4+ and/or CD8+ cells. In some embodiments, the cytokine is selected from IL-2, IL-15, IL-12, IL-7, IL-21, IL-18, TNF, and IFN-gamma.

As used herein, the term “cytokines” includes fusion proteins which comprise one or more cytokines, which are fused through a peptide linked to another cytokine or other immune modulatory molecule. Examples include but are not limited to IL-12 and IL-15 fusion proteins. In general, all agonists and antagonists described herein may be fused to another polypeptide of interest through a peptide linker, to improve or alter their function. Non-limiting examples of such fusion proteins include one or more cytokine polypeptides operably linked to an antibody polypeptide, wherein the antibody recognizes a tumor-specific antigen, thereby bringing the cytokine(s) into proximity with the tumor.

Interleukin 12 (IL-12) is a cytokine, the actions of which create an interconnection between the innate and adaptive immunity. IL-12 is secreted by a number of immune cells, including activated dendritic cells, monocytes, macrophages, and neutrophils, as well as other cell types. IL-12 is a heterodimeric protein (IL-12-p70; IL-12-p35/p40) consisting of p35 and p40 subunits, and binds to a receptor composed of two subunits, IL-12R-β1 and IL-12R-β2. IL-12 receptor is expressed constitutively or inducibly on a number of immune cells, including NK cells, T, and B lymphocytes. Upon binding of IL-12, the receptor is activated and downstream signaling through the JAK/STAT pathway initiated, resulting in the cellular response to IL-12. IL-12 acts by increasing the production of IFN-γ, which is the most potent mediator of IL-12 actions, from NK and T cells. In addition, IL-12 promotes growth and cytotoxicity of activated NK cells, CD8+ and CD4+ T cells, and shifts the differentiation of CD4+Th0 cells toward the Th1 phenotype. Further, IL-12 enhances of antibody-dependent cellular cytotoxicity (ADCC) against tumor cells and the induction of IgG and suppression of IgE production from B cells. In addition, IL-12 also plays a role in reprogramming of myeloid-derived suppressor cells, directs the Th1-type immune response and helps increase expression of MHC class I molecules (e.g., reviewed in Waldmann et al., Cancer Immunol Res March 2015 3; 219).

Thus, in some embodiments, the immune modulator is IL-12. In some embodiments, the IL-12 comprises the p35 and p40 subunits. In some embodiments, the interleukin-12 monomer subunits (IL-12A (p35) and IL-12B (p40)) are covalently linked by a linker. In some embodiments, the linker is a serine glycine rich linker. In one embodiment, the 15 amino acid linker of ‘GGGGSGGGGSGGGGS’ (SEQ ID NO: 1247) is inserted between two monomer subunits (IL-12A (p35) and IL-12B (p40) to produce a forced dimer human IL-12 (diIL-12) fusion protein.

IL-15 displays pleiotropic functions in homeostasis of both innate and adaptive immune system and binds to IL-15 receptor, a heterotrimeric receptor composed of three subunits. The alpha subunit is specific for IL-15, while beta (CD122) and gamma (CD132) subunits are shared with the IL-2 receptor, and allow shared signaling through the JAK/STAT pathways. IL-15 is produced by several cell types, including dendritic cells, monocytes and macrophages. Co-expression of IL-15Rα and IL-15 produced in the same cell, allows intracellular binding of IL-15 to IL-15Rα, which is then shuttled to the cell surface as a complex. Once on the cell surface, then, the IL-15Ra of these cells is able to trans-present IL-15 to IL-15Rβ-γc of CD8 T cells, NK cells, and NK-T cells, which do not express IL-15, inducing the formation of the so-called immunological synapse. Murine and human IL-15Rα, exists both in membrane bound, and also in a soluble form. Soluble IL-15Ra (sIL-15Rα) is constitutively generated from the transmembrane receptor through proteolytic cleavage.

IL-15 is critical for lymphoid development and peripheral maintenance of innate immune cells and immunological memory of T cells, in particular natural killer (NK) and CD8+ T cell populations. In contrast to IL-2, IL-15 does not promote the maintenance of Tregs and furthermore, IL-15 has been shown to protect effector T cells from IL-2-mediated activation-induced cell death.

Consequently, delivery of IL-15 is considered a promising strategy for long-term anti-tumor immunity. In a first-in-human clinical trial of recombinant human IL-15, a 10-fold expansion of NK cells and significantly increased the proliferation of γδT cells and CD8+ T cells was observed upon treatment. In addition, IL-15 superagonists containing cytokine-receptor fusion complexes have been developed and are evaluated to increase the length of the response. These include the L-15 N72D superagonist/IL-15RαSushi-Fc fusion complex (IL-15SA/IL-15RαSu-Fc; ALT-803) (Kim et al., 2016 IL-15 superagonist/IL-15RαSushi-Fc fusion complex (IL-15SA/IL-15RαSu-Fc; ALT-803) markedly enhances specific subpopulations of NK and memory CD8+ T cells, and mediates potent anti-tumor activity against murine breast and colon carcinomas).

Thus, in some embodiments, the immune modulator is IL-15.

The biological activity of IL-15 is greatly improved by pre-associating IL-15 with a fusion protein IL-15Rα-Fc or by direct fusion with the sushi domain of IL-15Rα (hyper-IL-15) to mimic trans-presentation of IL-15 by cell-associated IL-15Rα. IL-15, either administrated alone or as a complex with IL-15Rα, exhibits potent antitumor activities in animal models (Cheng et al., Immunotherapy of metastatic and autochthonous liver cancer with IL-15/IL-15Rα fusion protein; Oncoimmunology. 2014; 3(11): e963409, and references therein).

In some embodiments, the immune modulator is IL-15. In some embodiments, the immune modulator is IL-15Ra.

Interferon gamma (IFNγ or type II interferon), is a cytokine that is critical for innate and adaptive immunity against viral, some bacterial and protozoal infections. IFNγ activates macrophages and induces Class II major histocompatibility complex (MHC) molecule expression. IFNγ can inhibit viral replication and has immunostimulatory and immunomodulatory effects in the immune system. IFNγ is produced predominantly by natural killer (NK) and natural killer T (NKT) cells as part of the innate immune response, and by CD4 Th1 and CD8 cytotoxic T lymphocyte (CTL) effector T cells. Once antigen-specific immunity develops IFNγ is secreted by T helper cells (specifically, Th1 cells), cytotoxic T cells (TC cells) and NK cells only. It has numerous immunostimulatory effects and plays several different roles in the immune system, including the promotion of NK cell activity, increased antigen presentation and lysosome activity of macrophages, activation of inducible Nitric Oxide Synthase iNOS, production of certain IgGs from activated plasma B cells, promotion of Th1 differentiation that leads to cellular immunity. It can also cause normal cells to increase expression of class I MHC molecules as well as class II MHC on antigen-presenting cells, promote adhesion and binding relating to leukocyte migration, and is involved in granuloma formation through the activation of macrophages so that they become more powerful in killing intracellular organisms. Thus, in one embodiment, the immune modulator is IFN-gamma.

Interleukin-18 (IL-18, also known as interferon-gamma inducing factor) is a proinflammatory cytokine that belongs to the IL-1 superfamily and is produced by macrophages and other cells. IL-18 binds to the interleukin-18 receptor, and together with IL-12 it induces cell-mediated immunity following infection with microbial products like lipopolysaccharide (LPS). Upon stimulation with IL-18, natural killer (NK) cells and certain T helper type 1 cells release interferon-γ (IFN-γ) or type II interferon, which plays a role in activating the macrophages and other immune cells. IL-18 is also able to induce severe inflammatory reactions. Thus, in some embodiments, the immune modulator is IL-18.

Interleukin-2 (IL-2) is cytokine that regulates the activities of white blood cells (leukocytes, often lymphocytes). IL-2 is part of the body's natural response to microbial infection, and in discriminating between foreign (“non-self”) and “self”. IL-2 mediates its effects by binding to IL-2 receptors, which are expressed by lymphocytes. IL-2 is a member of a cytokine family, which also includes IL-4, IL-7, IL-9, IL-15 and IL-21. IL-2 signals through the IL-2 receptor, a complex consisting of alpha, beta and gamma sub-units. The gamma sub-unit is shared by all members of this family of cytokine receptors. IL-2 promotes the differentiation of T cells into effector T cells and into memory T cells when the initial T cell is stimulated by an antigen. Through its role in the development of T cell immunologic memory, which depends upon the expansion of the number and function of antigen-selected T cell clones, it also has a key role in cell-mediated immunity. IL-2 has been approved by the Food and Drug Administration (FDA) and in several European countries for the treatment of cancers (malignant melanoma, renal cell cancer). IL-2 is also used to treat melanoma metastases and has a high complete response rate. Thus, in some embodiments, the immune modulator is IL-2.

Interleukin-21 is a cytokine that has potent regulatory effects on certain cells of the immune system, including natural killer (NK) cells and cytotoxic T cells. IL-21 induces cell division/proliferation in its these cells. IL-21 is expressed in activated human CD4+ T cells but not in most other tissues. In addition, IL-21 expression is up-regulated in Th2 and Th17 subsets of T helper cells. IL-21 is also expressed in NK T cells regulating the function of these cells. When bound to IL-21, the IL-21 receptor acts through the Jak/STAT pathway, utilizing Jak1 and Jak3 and a STAT3 homodimer to activate its target genes. IL-21 has been shown to modulate the differentiation programming of human T cells by enriching for a population of memory-type CTL with a unique CD28+CD127hi CD45RO+ phenotype with IL-2 producing capacity. IL-21 also has anti-tumor effects through continued and increased CD8+ cell response to achieve enduring tumor immunity. IL-21 has been approved for Phase 1 clinical trials in metastatic melanoma (MM) and renal cell carcinoma (RCC) patients. Thus, in some embodiments, the immune modulator is IL-21.

Tumor necrosis factor (TNF) (also known as cachectin or TNF alpha) is a cytokine that can cause cytolysis of certain tumor cell lines and can stimulate cell proliferation and induce cell differentiation under certain conditions. TNF is involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. The primary role of TNF is in the regulation of immune cells.

TNF can bind two receptors, TNFR1 (TNF receptor type 1; CD120a; p55/60) and TNFR2 (TNF receptor type 2; CD120b; p75/80). TNFR1 is expressed in most tissues, and can be fully activated by both the membrane-bound and soluble trimeric forms of TNF, whereas TNFR2 is found only in cells of the immune system, and respond to the membrane-bound form of the TNF homotrimer. Upon binding to its receptor, TNF can activate NF-κB and MAPK pathways which mediate the transcription of numerous proteins and mediate several pathways involved in cell differentiation and proliferation, including those pathways involved in the inflammatory response. TNF also regulates pathways that induce cell apoptosis. Thus, in some embodiments, immune modulator modulates dendritic cell activation. In some embodiments, the immune modulator is TNF.

In some embodiments, the TNF is capable of increasing CCR7 expression on dendritic cells and/or macrophages.

In some embodiments, the TNFα is capable of activating the NFkappaB pathway, e.g., in cells with TNF receptor. In some embodiments, the TNFα is capable of inducing IkappaBalpha degradation. In some embodiments, TNFα is causes IkappaBalpha degradation.

In some embodiments, the immune modulator may be any one or more of the described IL-2, IL-15, IL-12, IL-7, IL-21, IL-18, TNF, and IFN-gamma.

In any of these combination embodiments, the bacteria administered with the immune modulator may further comprise an auxotrophic modification, e.g., a mutation or deletion in DapA, ThyA, or both. In any of these embodiments, the bacteria may further comprise a phage modification, e.g., a mutation or deletion, in an endogenous prophage as described herein.

### Pharmaceutical Compositions and Formulations

Pharmaceutical compositions comprising the microorganisms and/or immune modulators of the invention may be used to treat, manage, ameliorate, and/or prevent cancer. Pharmaceutical compositions of the invention may be used alone or in combination with prophylactic agents, therapeutic agents, and/or pharmaceutically acceptable carriers are provided.

In some embodiments, the bacteria are administered systemically or intratumorally as spores. As a non-limiting example, the bacteria are Clostridial strains, and administration results in a selective colonization of hypoxic/necrotic areas within the tumor. In some embodiments, the spores germinate exclusively in the hypoxic/necrotic regions present in solid tumors and nowhere else in the body.

The pharmaceutical compositions of the invention may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tableting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The compositions may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intratumoral, peritumor, immediate-release, pulsatile-release, delayed-release, or sustained release). Suitable dosage amounts for the bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal.

The bacteria and/or immune modulator(s) may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the bacteria of the invention may be formulated in a solution of sodium bicarbonate, e.g., 1 molar solution of sodium bicarbonate (to buffer an acidic cellular environment, such as the stomach, for example).

The compositions may be administered intravenously, e.g., by infusion or injection. Alternatively, the compositions may be administered intratumorally and/or peritumorally. In other embodiments, the compositions may be administered intra-arterially, intramuscularly, or intraperitoneally. In some embodiments, the bacteria colonize about 20%, 30%, 40%, 50%, 60%, 70%, 80%, 90% or more of the tumor. In some embodiments, the bacteria and/or immune modulator(s) are co-administered with a PEGylated form of rHuPH20 (PEGPH20) or other agent in order to destroy the tumor septae in order to enhance penetration of the tumor capsule, collagen, and/or stroma.

The microorganisms and/or immune modulator(s) of the disclosure may be administered via intratumoral injection. Intratumoral injection may elicit a potent localized inflammatory response as well as an adaptive immune response against tumor cells. In some embodiments, the tumor is injected with an 18-gauge multipronged needle (Quadra-Fuse, Rex Medical). The injection site is aseptically prepared. If available, ultrasound or CT may be used to identify a necrotic region of the tumor for injection. If a necrotic region is not identified, the injection can be directed to the center of the tumor. The needle is inserted once into a predefined region, and dispensed with even pressure. The injection needle is removed slowly, and the injection site is sterilized.

Direct intratumoral injection of the compositions of the invention into solid tumors may be advantageous as compared to intravenous administration. Using an intravenous injection method, only a small proportion of the bacteria may reach the target tumor. For example, following E. coli Nissle injection into the tail vein of 4T1 tumor-bearing mice, most bacteria (>99%) are quickly cleared from the animals and only a small percentage of the administered bacteria colonize the tumor (Stritzker et al., 2007). In particular, in large animals and human patients, which have relatively large blood volumes and relatively small tumors compared to mice, intratumoral injection may be especially beneficial. Injection directly into the tumor allows the delivery of a higher concentration of therapeutic agent and avoids the toxicity, which can result from systemic administration. In addition, intratumoral injection of bacteria induces robust and localized immune responses within the tumor.

Depending on the location, tumor type, and tumor size, different administration techniques may be used, including but not limited to, cutaneous, subcutaneous, and percutaneous injection, therapeutic endoscopic ultrasonography, or endobronchial intratumor delivery. Prior to the intratumor administration procedures, sedation in combination with a local anesthetic and standard cardiac, pressure, and oxygen monitoring, or full anesthesia of the patient is performed.

For some tumors, percutaneous injection can be employed, which is the least invasive administration method. Ultrasound, computed tomography (CT) or fluoroscopy can be used as guidance to introduce and position the needle. Percutaneous intratumoral injection is for example described for hepatocellular carcinoma in Lencioni et al., 2010. Intratumoral injection of cutaneous, subcutaneous, and nodal tumors is for example described in WO/2014/036412 (Amgen) for late stage melanoma.

Single insertion points or multiple insertion points can be used in percutaneous injection protocols. Using a single insertion point, the solution may be injected percutaneously along multiple tracks, as far as the radial reach of the needle allows. In other embodiments, multiple injection points may be used if the tumor is larger than the radial reach of the needle. The needle can be pulled back without exiting, and redirected as often as necessary until the full dose is injected and dispersed. To maintain sterility, a separate needle is used for each injection. Needle size and length varies depending on the tumor type and size.

In some embodiments, the tumor is injected percutaneously with an 18-gauge multipronged needle (Quadra-Fuse, Rex Medical). The device consists of an 18 gauge puncture needle 20 cm in length. The needle has three retractable prongs, each with four terminal side holes and a connector with extension tubing clamp. The prongs are deployed from the lateral wall of the needle. The needle can be introduced percutaneously into the center of the tumor and can be positioned at the deepest margin of the tumor. The prongs are deployed to the margins of the tumor. The prongs are deployed at maximum length and then are retracted at defined intervals. Optionally, one or more rotation-injection-rotation maneuvers can be performed, in which the prongs are retracted, the needle is rotated by a 60 degrees, which is followed by repeat deployment of the prongs and additional injection.

Therapeutic endoscopic ultrasonography (EUS) is employed to overcome the anatomical constraints inherent in gaining access to certain other tumors (Shirley et al., 2013). EUS-guided fine needle injection (EUS-FNI) has been successfully used for antitumor therapies for the treatment of head and neck, esophageal, pancreatic, hepatic, and adrenal masses (Verna et al, 2008). EUS-FNI has been extensively used for pancreatic cancer injections. Fine-needle injection requires the use of the curvilinear echoendoscope. The esophagus is carefully intubated and the echoendoscope is passed into the stomach and duodenum where the pancreatic examination occurs, and the target tumor is identified. The largest plane is measured to estimate the tumor volume and to calculate the injection volume. The appropriate volume is drawn into a syringe. A primed 22-gauge fine needle aspiration (FNA) needle is passed into the working channel of the echoendoscope. Under ultrasound guidance, the needle is passed into the tumor. Depending on the size of the tumor, administration can be performed by dividing the tumor into sections and then injecting the corresponding fractions of the volume into each section. Use of an installed endoscopic ultrasound processor with Doppler technology assures there are no arterial or venous structures that may interfere with the needle passage into the tumor (Shirley et al., 2013). In some embodiments, ‘multiple injectable needle’ (MIN) for EUS-FNI can be used to improvement the injection distribution to the tumor in comparison with straight-type needles (Ohara et al., 2013).

Intratumoral administration for lung cancer, such as non-small cell lung cancer, can be achieved through endobronchial intratumor delivery methods, as described in Celikoglu et al., 2008. Bronchoscopy (trans-nasal or oral) is conducted to visualize the lesion to be treated. The tumor volume can be estimated visually from visible length-width height measurements over the bronchial surface. The needle device is then introduced through the working channel of the bronchoscope. The needle catheter, which consists of a metallic needle attached to a plastic catheter, is placed within a sheath to prevent damage by the needle to the working channel during advancement. The needle size and length varies and is determined according to tumor type and size of the tumor. Needles made from plastic are less rigid than metal needles and are ideal, since they can be passed around sharper bends in the working channel. The needle is inserted into the lesion and the bacteria of the invention are in injected. Needles are inserted repeatedly at several insertion points until the tumor mass is completely perfused. After each injection, the needle is withdrawn entirely from the tumor and is then embedded at another location. At the end of the bronchoscopic injection session, removal of any necrotic debris caused by the treatment may be removed using mechanical dissection, or other ablation techniques accompanied by irrigation and aspiration.

In some embodiments, the compositions are administrated directly into the tumor using methods, including but not limited to, percutaneous injection, EUS-FNI, or endobronchial intratumor delivery methods. In some cases, other techniques, such as laparoscopic or open surgical techniques are used to access the target tumor, however, these techniques are much more invasive and bring with them much greater morbidity and longer hospital stays.

In some embodiments, bacteria, e.g., E. coli Nissle, or spores, e.g., Clostridium novyi NT, are dissolved in sterile phosphate buffered saline (PBS) for systemic or intratumor injection.

The dose to be injected is derived from the type and size of the tumor. The dose of a drug or the bacteria is typically lower, e.g., orders of magnitude lower, than a dose for systemic intravenous administration.

The volume injected into each lesion is based on the size of the tumor. To obtain the tumor volume, a measurement of the largest plane can be conducted. The estimated tumor volume can then inform the determination of the injection volume as a percentage of the total volume. For example, an injection volume of approximately 20-40% of the total tumor volume can be used.

For example, as is for example described in WO/2014/036412, for tumors larger than 5 cm in their largest dimension, up to 4 ml can be injected. For tumors between 2.5 and 5 cm in their largest dimension, up to 2 ml can be injected. For tumors between 2.5 and 5 cm in their largest dimension, up to 2 ml can be injected. For tumors between 1.5 and 2.5 cm in their largest dimension, up to 1 ml can be injected. For tumors between 0.5 and 1.5 cm in their largest dimension, up to 0.5 ml can be injected. For tumors equal or small than 0.5 in their largest dimension, up to 0.1 ml can be injected. Alternatively, ultrasound scan can be used to determine the injection volume that can be taken up by the tumor without leakage into surrounding tissue.

In some embodiments, the treatment regimen will include one or more intratumoral administrations. In some embodiments, a treatment regimen will include an initial dose, which followed by at least one subsequent dose. One or more doses can be administered sequentially in two or more cycles.

For example, a first dose may be administered at day 1, and a second dose may be administered after 1, 2, 3, 4, 5, 6, days or 1, 2, 3, or 4 weeks or after a longer interval. Additional doses may be administered after 1, 2, 3, 4, 5, 6, days or after 1, 2, 3, or 4 weeks or longer intervals. In some embodiments, the first and subsequent administrations have the same dosage. In other embodiments, different doses are administered. In some embodiments, more than one dose is administered per day, for example, two, three or more doses can be administered per day.

The routes of administration and dosages described are intended only as a guide. The optimum route of administration and dosage can be readily determined by a skilled practitioner. The dosage may be determined according to various parameters, especially according to the location of the tumor, the size of the tumor, the age, weight and condition of the patient to be treated and the route and method of administration.

In some embodiments, the bacteria is administered via first route, e.g., intratumoral injection, and the at least one immune modulator is administered via a second route, e.g., orally.

In some embodiments, the compositions of the disclosure may be administered orally. In some embodiments, the compositions may be useful in the prevention, treatment or management of liver cancer or liver metastases. For example, Danino et al showed that orally administered E. coli Nissle is able to colonize liver metastases by crossing the gastrointestinal tract in a mouse model of liver metastases (Danino et al., Programmable probiotics for detection of cancer in urine. Science Translational Medicine, 7 (289): 1-10, the contents of which is herein incorporated by reference in its entirety).

In one embodiment, the composition is delivered by intratumor injection. In one embodiment, the composition is delivered intrapleurally. In one embodiment, the composition is delivered subcutaneously. In one embodiment, the composition is delivered intravenously. In one embodiment, the composition is delivered intrapleurally.

In some embodiments, the compositions may be administered intratumorally according to a regimen which requires multiple injections. In some embodiments, the bacteria and at least one immune modulator are administered together in each intratumoral injection. In some embodiments, a bacteria strain is injected first and an immune modulator is injected at a later timepoint. In other embodiments, an immune modulator is injected first, and a bacteria is injected at a later time point. Additional injections, either concurrently or sequentially, can follow.

Tumor types into which the bacteria of the current invention are intratumorally delivered include locally advanced and metastatic tumors, including but not limited to, B, T, and NK cell lymphomas, colon and rectal cancers, melanoma, including metastatic melanoma, mycosis fungoides, Merkel carcinoma, liver cancer, including hepatocellular carcinoma and liver metastasis secondary to colorectal cancer, pancreatic cancer, breast cancer, follicular lymphoma, prostate cancer, refractory liver cancer, and Merkel cell carcinoma.

In some embodiments, tumor cell lysis occurs as part of the intratumor injection. As result, tumor antigens may exposed eliciting an anti-tumor response. This exposure may work together with the effector expressed by the bacteria to enhance the anti-tumor effect. In some embodiments, tumor cell lysis does not occur as part of the intratumor injection.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the composition is formulated for intravenous administration, intratumor administration, or peritumor administration. The composition may be formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

### Methods of Treatment

Another aspect of the invention provides methods of treating cancer. In some embodiments, the invention provides methods for reducing, ameliorating, or eliminating one or more symptom(s) associated with cancer. In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, laryngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer (e.g., small cell lung cancer, non-small cell lung cancer), lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma (e.g., liposarcoma, chondrosarcoma), skin cancer (e.g., basal cell carcinoma, melanoma, squamous cell carcinoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macroglobulinemia, and Wilms tumor. In some embodiments, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

The method may comprise preparing a pharmaceutical composition with at least one species, strain, or subtype of bacteria and/or immune modulator described herein, and administering the pharmaceutical composition to a subject in a therapeutically effective amount. The composition may be administered locally, e.g., intratumorally or peritumorally into a tissue or supplying vessel, or systemically, e.g., intravenously by infusion or injection. In some embodiments, the compositions are administered intravenously, intratumorally, intra-arterially, intramuscularly, intraperitoneally, orally, or topically. In some embodiments, the compositions are administered intravenously, i.e., systemically.

In some embodiments, the recombinant microorganism is administered to the subject at a suitable dose. Examples include administered to the subject at a dose of about 1×10⁶, about 3×10⁶, about 1×10⁷, about 3×10⁷, about 1×10⁸, about 3×10⁸, and about 1×10⁹ live cells. In some embodiments, a suitable dose may be selected from about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, and from about 3×10⁸ to about 1×10⁹ live cells. Accordingly, methods for treating a solid tumor are provided herein, comprising administering to a subject in need thereof an effective amount of an a recombinant microorganism capable of expressing a stimulator of interferon gene (STING) agonist and wherein the microorganism is administered to the subject at a dose of about 1×10⁶, about 3×10⁶, about 1×10⁷, about 3×10⁷, about 1×10⁸, about 3×10⁸, and about 1×10¹ live cells. In some embodiments, a suitable dose may be selected from about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, and from about 3×10⁸ to about 1×10⁹ live cells. In some embodiments, recombinant microorganism is administered to the subject once weekly, once every two weeks, or once every three weeks. In some embodiments, the recombinant microorganism is administered to the subject once weekly. In some embodiments, the recombinant microorganism is administered to the subject once every three weeks. In some embodiments, the administering is intratumoral injection. In some embodiments, the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months. In some embodiments, the recombinant microorganism is administered for at least 24 months. One specific embodiment, the microorganism is administered in four 21-day cycles. In some embodiments, the number of administrations differs between cycles. For example, a subject may receive a dose, e.g., intratumorally, of the microorganism on days 1, 8, and 15 of Cycle 1 and Day 1 of Cycles 2 through 4. In some embodiments, the solid tumor is a lymphoma, melanoma, liposarcoma, sarcoma, small cell lung cancer, squamous cell carcinoma, or chondrosarcoma. In some embodiments, the STING agonist is c-diAMP, c-GAMP, or c-diGMP. In some embodiments, the STING agonist is c-diAMP. In some embodiments, the recombinant microorganism expresses at least one non-native gene sequence encoding an enzyme capable of producing the STING agonist. In some embodiments, the at least one non-native gene sequence is dacA or cGAS. In some embodiments, the non-native gene sequence is integrated into a chromosome of the microorganism. Alternatively, the at least one non-native gene sequence is present on a plasmid in the microorganism. In some embodiments, the at least one non-native gene sequence encoding the enzyme capable of producing the STING agonist is operably linked to a constitutive promoter. In some embodiments, the at least one non-native gene sequence encoding the enzyme capable of producing the STING agonist is operably linked to an inducible promoter, for example, a low-oxygen or anaerobic conditions, by the hypoxic environment of a tumor, or temperature. In some embodiments, the recombinant microorganism further comprises one or more auxotrophies and/or one or more endogenous phage deletions. In one embodiment, the recombinant microorganism is an auxotroph in dapA, thyA, or both dapA and thyA. In one embodiment, the recombinant microorganism is non-pathogenic to the subject. In one example, the recombinant microorganism is Escherichia coli Nissle. In any of these embodiments, the methods may further provide co-administration with a checkpoint inhibitor, e.g., an anti-Pd1 antibody such as atezolizumab, as described herein.

In certain embodiments, administering the pharmaceutical composition to the subject reduces cell proliferation, tumor growth, and/or tumor volume in a subject. In some embodiments, the methods of the present disclosure may reduce cell proliferation, tumor growth, and/or tumor volume by at least about 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to levels in an untreated or control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more.

Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In some embodiments, the methods may include administration of the compositions of the invention to reduce tumor volume in a subject to an undetectable size, or to less than about 1%, 2%, 5%, 10%, 20%, 25%, 30%, 40%, 50%, 60%, 70%, 75%, 80%, or 90% of the subject's tumor volume prior to treatment. In other embodiments, the methods may include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

In certain embodiments, administering the pharmaceutical composition to the subject upregulates cytokines found in blood and serum. In some embodiments, the blood and serum cytokines are IL-6, TNFα, IFNγ, and/or IL-1Ra. In some embodiments, the serum and blood cytokines are upregulated in a dose dependent manner. In other embodiments, serum and blood cytokines are upregulated about 2-fold, about 3-fold, about 4-fold, or about 5-fold more than expression prior to treatment.

In certain embodiments, administering the pharmaceutical composition to the subject upregulates expression of interferon stimulated genes, chemokine and/or cytokine genes, and T cell function genes. In some embodiments the interferon stimulated genes are ISG15 (interferon stimulated gene), IFIT1, and/or IFIT2, the chemokine and/or cytokines are CXCL9, CXCL10, TNFRS1B, and/or TNFSF10, and the T cell function genes are Granzyme A (GZMA), CD4, PD-L2. In some embodiments, interferon stimulated genes, chemokine and/or cytokine genes, and T cell function genes are upregulated about 2-fold, about 3-fold, or about 4-fold.

Response patterns may be different than for traditional cytotoxic therapies. For example, tumors treated with immune-based therapies may enlarge before they regress, and/or new lesions may appear (Agarwala et al., 2015). Increased tumor size may be due to heavy infiltration with lymphocytes and macrophages that are normally not present in tumor tissue. Additionally, response times may be slower than response times associated with standard therapies, e.g., cytotoxic therapies. In some embodiments, delivery of the immune modulator may modulate the growth of a subject's tumor and/or ameliorate the symptoms of a cancer while temporarily increasing the volume and/or size of the tumor.

The recombinant bacteria may be destroyed, e.g., by defense factors in tissues or blood serum (Sonnenborn et al., 2009), or by activation of a kill switch, several hours or days after administration. Thus, the pharmaceutical composition may be re-administered at a therapeutically effective dose and frequency. In alternate embodiments, the bacteria are not destroyed within hours or days after administration and may propagate in the tumor and colonize the tumor.

The pharmaceutical composition may be administered alone or in combination with one or more additional therapeutic agents, e.g., a chemotherapeutic drug or a checkpoint inhibitor, e.g., as described herein and known in the art. An important consideration in selecting the one or more additional therapeutic agents is that the agent(s) should be compatible with the bacteria of the invention, e.g., the agent(s) must not kill the bacteria. In some studies, the efficacy of anticancer immunotherapy, e.g., CTLA-4 or PD-1 inhibitors, requires the presence of particular bacterial strains in the microbiome (Ilda et al., 2013; Vetizou et al., 2015; Sivan et al., 2015). In some embodiments, the pharmaceutical composition comprising the bacteria augments the effect of a checkpoint inhibitor or a chemotherapeutic agent, e.g., allowing lowering of a the dose of systemically administrated chemotherapeutic or immunotherapeutic agents. In some embodiments, the pharmaceutical composition is administered with one or more commensal or probiotic bacteria, e.g., Bifidobacterium or Bacteroides.

In certain embodiments, the pharmaceutical composition may be administered to a subject for treating cancer by administering a bacterium to the subject, and administering at least one immune modulator to the subject. In some embodiments, the administering steps are performed at the same time. In some embodiments, administering the bacterium to the subject occurs before the administering of the at least one immune modulator to the subject. In some embodiments, administering of the at least one immune modulator to the subject occurs before the administering of the bacterium to the subject.

In some embodiments, subject to be treated by the methods disclosed herein meets one or more of the inclusion and exclusion criteria disclosed the examples below. For example, the subject may be (1) aged ≥18 years having a histologically- or cytologically-confirmed stage III or IV advanced/metastatic solid tumor or lymphoma for which no therapeutic options are available to extend survival or for which the patient is not a candidate for standard-of-care therapy, (2) having Eastern Cooperative Oncology Group performance status ≤1, (3) having a life expectancy ≥3 months, (4) having lesions ≥1 injectable, measurable (≥10 mm in diameter, or ≥15 mm for nodal lesions), having an eligible lesion as defined by RECIST 1.1 (Eisenhauer et al 2009), iRECIST (Seymour et al 2017), and/or LYRIC (Cheson et al 2016) and as assessed by the Investigator. (5) having Oxygen saturation ≥90% without the use of supplemental oxygen, (6) having adequate cardiac function. Alternatively or in addition the subject may have laboratory values within the following ranges: Absolute neutrophil count ≥1500/μL; Lymphocyte count ≥500/μL; Platelets ≥100,000/μL without transfusion; Hemoglobin ≥9.0 g/dL (patients may be transfused to meet this criterion); Estimated glomerular filtration rate (eGFR) ≥50 m/min/1.73 m2 per the Cockcroft-Gault formula; Total bilirubin ≤1.5×the upper limit of normal (ULN) OR direct bilirubin ≤ULN for participants with total bilirubin levels ≥1.5×ULN or ≤3×ULN for patients with Gilbert's syndrome; AST, ALT, and alkaline phosphatase ≤2.5×ULN (AST and ALT≤5×ULN for participants with liver metastases and alkaline phosphatase ≤5×ULN for participants with liver or bone metastases); International normalized ratio (INR) or PT or aPTT≤1.5×ULN unless participant is receiving anticoagulant therapy as long as PT or aPTT is within therapeutic range of intended use of anticoagulants.

When the microorganism is combined with checkpoint inhibitorthereay, additional inclusion criteria may apply. For example, TSH must be within the normal reference range or the subject must be receiving stable thyroid replacement therapy. In some embodiments, Patients have received previous treatment with an anti-PD-1/L1 monoclonal antibody (mAb) administered either as monotherapy, or in combination with other CPIs or other therapies. In some embodiments, e.g., if CPI therapy is not indicated as a part of standard-of-care therapy, the subject may be CPI naïve.

Additional inclusion criteria that apply only to the microorganism-CPI combination therapy include having progressed on treatment with an anti-PD-1/L1 mAb administered either as monotherapy, or in combination with other CPIs or other therapies, if indicated or if CPI therapy is not indicated as a part of standard-of-care therapy, being be CPI naïve.

### Chemotherapeutic Agents

In some embodiments, the pharmaceutical compositions are administered sequentially, simultaneously, or subsequently to dosing with one or more chemotherapeutic agents. In some embodiments, the pharmaceutical compositions are administered sequentially, simultaneously, or subsequently to dosing with one or more chemotherapeutic agents selected from Trabectedin®, Belotecan®, Cisplatin®, Carboplatin®, Bevacizumab®, Pazopanib®, 5-Fluorouracil, Capecitabine®, Irinotecan®, and Oxaliplatin®. In some embodiments, the pharmaceutical compositions are administered sequentially, simultaneously, or subsequently to dosing with gemcitabine (Gemzar). In some embodiments, the pharmaceutical compositions are administered sequentially, simultaneously, or subsequently to dosing with cyclophosphamide. In any of these embodiments, the one or more bacteria are administered systemically or orally or intratumorally.

In some embodiments, one or more pharmaceutical compositions are administered sequentially, simultaneously, or subsequently to dosing with one or more chemotherapeutic agents. In some embodiments, the chemotherapeutic agent is administered systemically, and the bacteria are administered intratumorally. In some embodiments, the chemotherapeutic agent and pharmaceutical composition are administered systemically. In one embodiment, the chemotherapeutic agent is cyclophosphamide.

In some embodiments, the pharmaceutical compositions are able to improve anti-tumor activity (e.g., tumor proliferation, size, volume, weight) of the co-administered chemotherapeutic agent (e.g., cyclophosphamide or another agent described herein or known in the art), e.g., by 10% to 20%, 20% to 25%, 25% to 30%, 30% to 40%, 40% to 50%, 50% to 60%, 60% to 70%, 70% to 75%, 75% to 80%, 80% to 85%, 85% to 90%, 90% to 95%, 95% to 99%, or more as compared to a chemotherapy alone under the same conditions. In some embodiments, the pharmaceutical compositions are able to improve anti-tumor activity (e.g., tumor proliferation, size, volume, weight) of the co-administered chemotherapeutic agent (e.g., cyclophosphamide or another agent described herein or known in the art), e.g., 1.0-1.2-fold, 1.2-1.4-fold, 1.4-1.6-fold, 1.6-1.8-fold, 1.8-2-fold, or two-fold more or more as compared to a chemotherapy alone.

### Checkpoint Inhibition

In certain embodiments, the pharmaceutical compositions are administered sequentially, simultaneously, or subsequently to dosing with one or more checkpoint inhibitors, immune stimulatory antibodies (inhibitory or agonistic) or other agonists known in the art or described herein. In certain embodiments, the pharmaceutical compositions are administered sequentially, simultaneously, or subsequently to dosing with one checkpoint inhibitors, immune stimulatory antibodies (inhibitory or agonistic) or other agonists known in the art or described herein. In certain embodiments, the pharmaceutical compositions are administered sequentially, simultaneously, or subsequently to dosing with two checkpoint inhibitors, immune stimulatory antibodies (inhibitory or agonistic) or other agonists known in the art or described herein.

Non-limiting examples of immune checkpoint inhibitors include CTLA-4 antibodies (including but not limited to Ipilimumab and Tremelimumab (CP675206)), anti-4-iBB (CD137, TNFRSF9) antibodies (including but not limited to PF-05082566, and Urelumab), anti CD134 (OX40) antibodies, including but not limited to Anti-OX40 antibody (Providence Health and Services), anti-PD-1 antibodies (including but not limited to Nivolumab, Pidilizumab, Pembrolizumab (MK-3475/SCH900475, lambrolizumab, REGN2810, PD-1 (Agenus)), dostarlimab, anti-PD-L1 antibodies (including but not limited to durvalumab (MEDI4736), avelumab (MSB0010718C), and atezolizumab (MPDL3280A, RG7446, R05541267)), and anti-KIR antibodies (including but not limited to Lirilumab), LAG3 antibodies (including but not limited to BMS-986016), anti-CCR4 antibodies (including but not limited to Mogamulizumab), anti-CD27 antibodies (including but not limited to Varlilumab), anti-CXCR4 antibodies (including but not limited to Ulocuplumab). In some embodiments, the at least one bacterial cell is administered sequentially, simultaneously, or subsequently to dosing with an anti-phosphatidyl serine antibody (including but not limited to Bavituxumab).

In some embodiments, the pharmaceutical compositions are administered sequentially, simultaneously, or subsequently to dosing with one or more antibodies selected from TLR9 antibody (including, but not limited to, MGN1703 PD-1 antibody (including, but not limited to, SHR-1210 (Incyte/Jiangsu Hengrui)), anti-OX40 antibody (including, but not limited to, OX40 (Agenus)), anti-Tim3 antibody (including, but not limited to, Anti-Tim3 (Agenus/INcyte)), anti-Lag3 antibody (including, but not limited to, Anti-Lag3 (Agenus/INcyte)), anti-B7H3 antibody (including, but not limited to, Enoblituzumab (MGA-271), anti-CT-011 (hBAT, hBAT1) as described in WO2009101611, anti-PDL-2 antibody (including, but not limited to, AMP-224 (described in WO2010027827 and WO2011066342)), anti-CD40 antibody (including, but not limited to, CP-870, 893), anti-CD40 antibody (including, but not limited to, CP-870, 893).

In one embodiment, the microorganism or the pharmaceutical composition comprising the microorganism is administered in combination with nivolumab. In some embodiments, nivolumab is administered at a flat dose of 240 mg every two weeks. In some embodiments, the microorganism is administered in combination with prembrolizumab. In some embodiments, prembrolizumab is administered at a flat doso of 400 mg every six weeks for pembrolizumab or alternatively at a flat does of 200 mg every three weeks.

In one embodiment, the microorganism or the pharmaceutical composition comprising the microorganism is administered in combination with atezolizumab. In one embodiment, atezolizumab is administered in accordance with its recommended dose and schedule. In one embodiment, atezolizumab is administered as a flat dose of 1200 mg as an intravenous infusion over 60 minutes every 3 weeks. For example, atezolizumab may be administered in accordance with its recommended dose and schedule (1200 mg IV Q3W) on Day 1 of each of cycles of administration of the recombinant microorganism. In some embodiments, the administration regimen comprises 4 cycles, e.g., of 21 days. In some embodiments, when a checkpoint inhibitor, e.g., atezolizumab, and the recombinanat microorganism are both administered on the same day, the recombinanat microorganism will be administered first, followed by at least 1 hour of observation prior to the checkpoint inhibitor infusion, e.g., atezolizumab infusion. In some instances, patients receiving a combination treatment with a checkpoint inhibitor, e.g., atezolizumab, and who do not have progressive disease (i.e., those who achieve and sustain CR, PR, or SD) at the end of Cycle 4 on combination therapy may receive additional cycles of SYNB1891 and atezolizumab for up to 24 months (i.e., Cycles 5 to 35) after the initial dose of study treatment until documentation of progressive disease or other discontinuation criteria, satisfaction of a predefined study stopping rule, or no eligible lesions remain.

Accordingly, methods for treating a solid tumor are provided herein, comprising administering to a subject in need thereof an effective amount of an a recombinant microorganism capable of expressing a stimulator of interferon gene (STING) agonist in combination with a checkpoint inhibitor, e.g., an anti-Pd1 antibody, e.g., atezolizumab, prembrolizomb or nivolumab or others known in the art and wherein the microorganism is administered to the subject at a dose of about 1×10⁶, about 3×10⁶, about 1×10⁷, about 3×10⁷, about 1×10⁸, about 3×10⁸, and about 1×10⁹ live cells. In some embodiments, a suitable dose may be selected from about 1×10⁶ to about 3×10⁶, fro 3×10⁶ to about 1×10⁷, from 1×10⁷, to about 3×10⁷, from 3×10⁷ to about 1×10⁸, from 1×10⁸ to about 3×10⁸, and from 3×10⁸ to about 1×10⁹ live cells. In some embodiments, recombinant microorganism is administered to the subject once weekly, once every two weeks, or once every three weeks. In some embodiments, the recombinant microorganism is administered to the subject once weekly. In some embodiments, the recombinant microorganism is administered to the subject once every three weeks. In some embodiments, the administering is intratumoral injection. In some embodiments, the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months. In some embodiments, the recombinant microorganism is administered for at least 24 months. One specific embodiment, the microorganism is administered in four 21-day cycles. In some embodiments, the number of administrations differs between cycles. For example, a subject may receive a dose, e.g., intratumorally, of the microorganism on days 1, 8, and 15 of Cycle 1 and Day 1 of Cycles 2 through 4.

### Co-Stimulatory Molecules

In certain embodiments, one or more bacteria and/or immune modulators are administered sequentially, simultaneously, or subsequently to dosing with one or more agonistic immune stimulatory molecules or agonists, including but not limited to, agonistic antibodies.

In some embodiments, the one or more antibodies are selected from anti-OX40 antibody (including, but not limited to, INCAGN01949 (Agenus); BMS 986178 (Bristol-Myers Squibb), MED10562 (Medimmune), GSK3174998 (GSK), PF-04518600 (Pfizer)), anti-41BB/CD137 (including but not limited to PF-05082566 (Pfizer), urelumab (BMS-663513; Bristol-Myers Squibb), and anti-GITR (including but not limited to TRX518 (Leap Therapeutics), MK-4166 (Merck), MK-1248 (Merck), AMG 228 (Amgen), BMS-986156 (BMS), INCAGN01876 (Incyte/Agenus), MEDI1873 (AZ), GWN323 (NVS).

In some embodiments, the microorganisms and/or immune modulators may be administered as part of a regimen, which includes other treatment modalities or combinations of other modalities. Non-limiting examples of these modalities or agents are conventional therapies (e.g., radiotherapy, chemotherapy), other immunotherapies, stem cell therapies, and targeted therapies, (e.g., BRAF or vascular endothelial growth factor inhibitors; antibodies or compounds), bacteria described herein, and oncolytic viruses. Therapies also include related to antibody-immune engagement, including Fc-mediated ADCC therapies, therapies using bispecific soluble scFvs linking cytotoxic T cells to tumor cells (e.g., BiTE), and soluble TCRs with effector functions. Immunotherapies include vaccines (e.g., viral antigen, tumor associated antigen, neoantigen, or combinations thereof), checkpoint inhibitors, cytokine therapies, adoptive cellular therapy (ACT). ACT includes but is not limited to, tumor infiltrating lymphocyte (TIL) therapies, native or engineered TCR or CAR-T therapies, natural killer cell therapies, and dendritic cell vaccines or other vaccines of other antigen presenting cells. Targeted therapies include antibodies and chemical compounds, and include for example antiangiogenic strategies and BRAF inhibition.

The immunostimulatory activity of bacterial DNA is mimicked by synthetic oligodeoxynucleotides (ODNs) expressing unmethylated CpG motifs. Bode et al., Expert Rev Vaccines. 2011 April; 10(4): 499-511. CpG DNA as a vaccine adjuvant. When used as vaccine adjuvants, CpG ODNs improve the function of professional antigen-presenting cells and boost the generation of humoral and cellular vaccine-specific immune responses. In some embodiments, CpG can be administered in combination with the bacteria of the invention.

In one embodiment, the microorganisms are administered in combination with tumor cell lysates.

The dosage of the pharmaceutical composition and the frequency of administration may be selected based on the severity of the symptoms and the progression of the cancer. The appropriate therapeutically effective dose and the frequency of administration can be selected by a treating clinician.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

The disclosure provides herein a sequence having at least about 80%, at least about 85%, at least about 90%, at least about 95%, or at least about 99% homologous to the sequence any of the SEQ ID NOs described in the Examples, below.

### Example 1. Intratumoral Injection of SYNB1891: Interim Clinical Trial Analysis

SYNB1891 is a live, modified strain of the probiotic E. coli Nissle engineered to produce cyclic dinucleotides under hypoxia leading to stimulator of interferon genes (STING)-activation in phagocytic antigen-presenting cells in tumors and activating complementary innate immune pathways. As used herein, SYNB comprises a bacterial chassis: a wild-type E. coli Nissle strain containing dual auxotrophies for diaminopimelic acid and thymidine, and deletion of an endogenous phage, and SYNB1891 comprises the SYNB Nissle strain plus an FNR-inducible dacA from Listeria monocytogenes integrated into the genome to produce the STING agonist ci-di-AMP.

This first-in-human study (NCT04167137; see Example 4 for details) is enrolling patients with refractory advanced solid tumors or lymphoma to receive an intratumoral (IT) injection of SYNB1891 on Days 1, 8 and 15 of the first 21-day cycle and then on Day 1 of each subsequent cycle. Dose escalation is planned across 7 cohorts (1×10⁶-1×10⁹ live cells) with Arm 1 consisting of escalating doses of SYNB1891 as monotherapy, and Arm 2 in combination with atezolizumab. The primary objective is to determine the single-agent maximum tolerated dose as monotherapy and the recommended Phase 2 dose in combination with atezolizumab. Other objectives include SYNB1891 kinetics in blood and the injected tumor, STING-target engagement as assessed by IT gene expression and serum cytokines, and tumor responses.

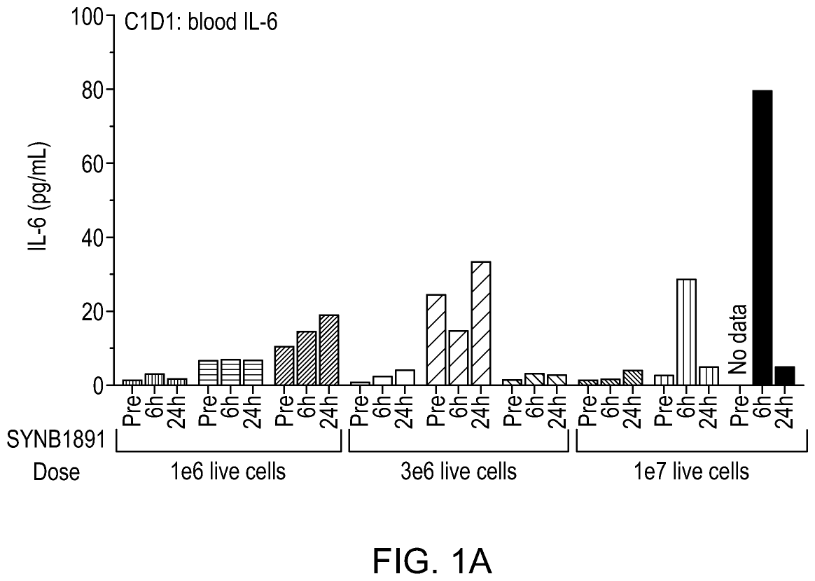

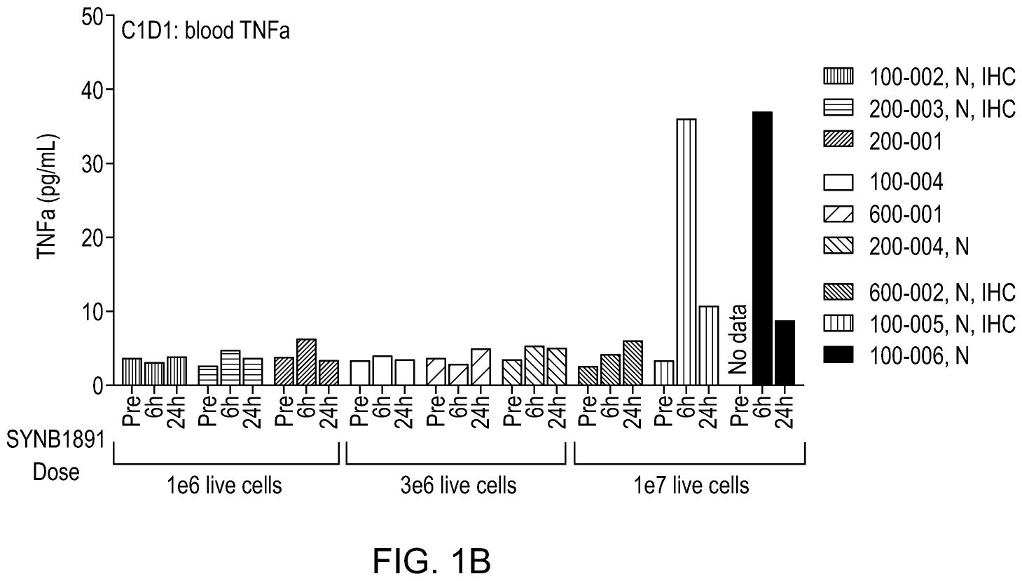

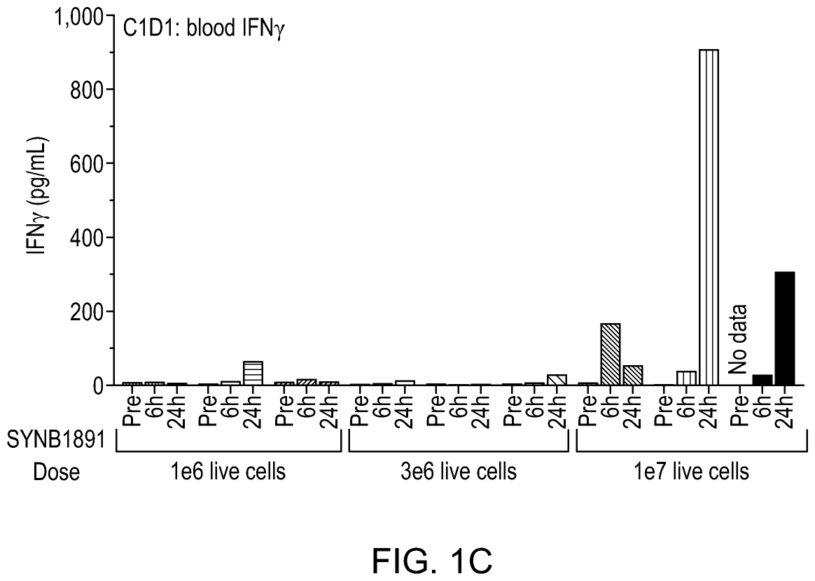

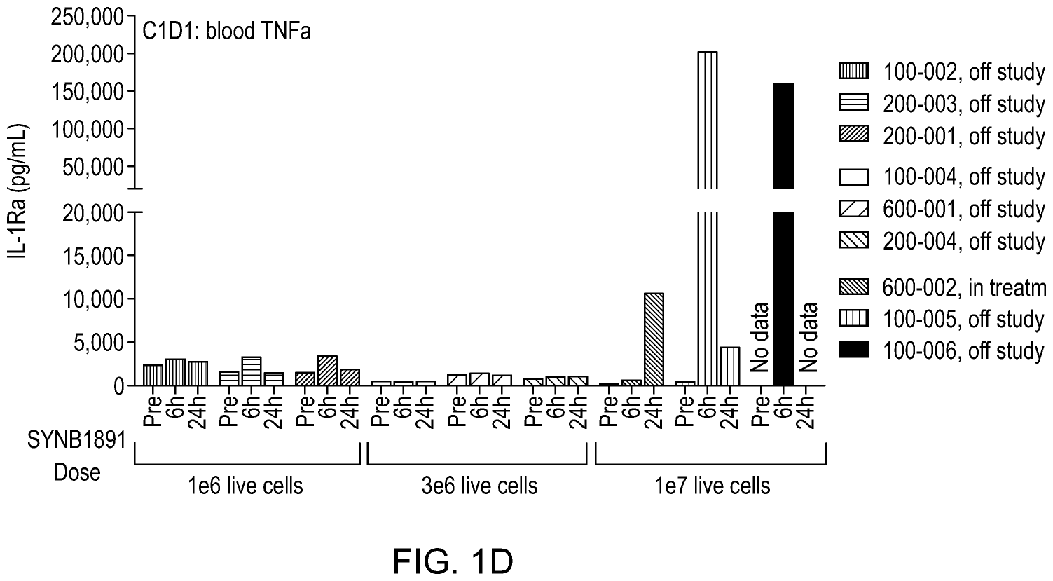

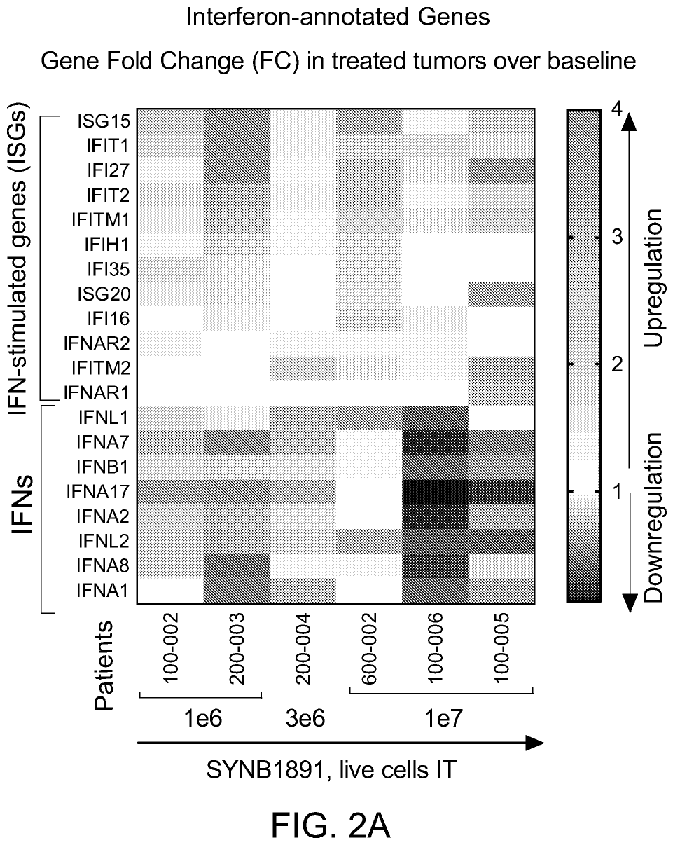

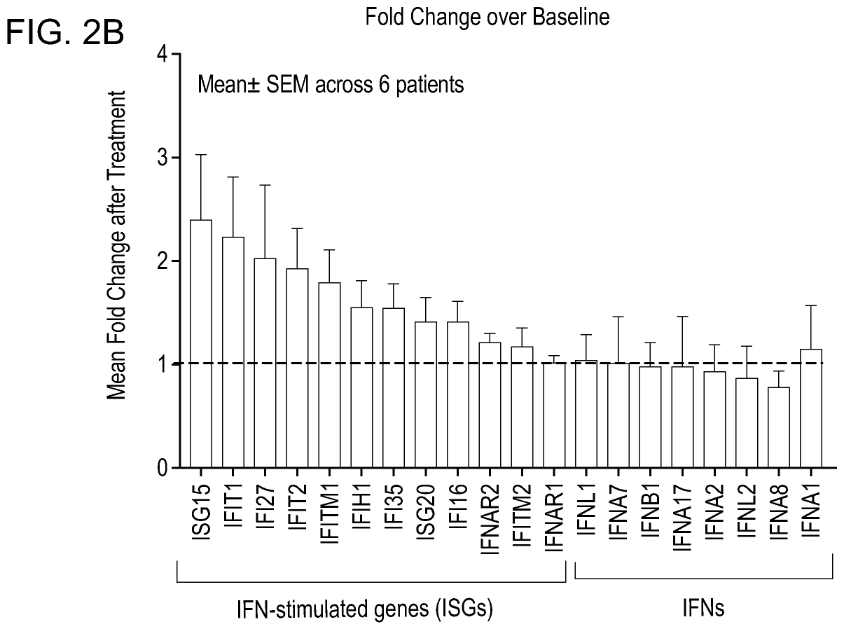

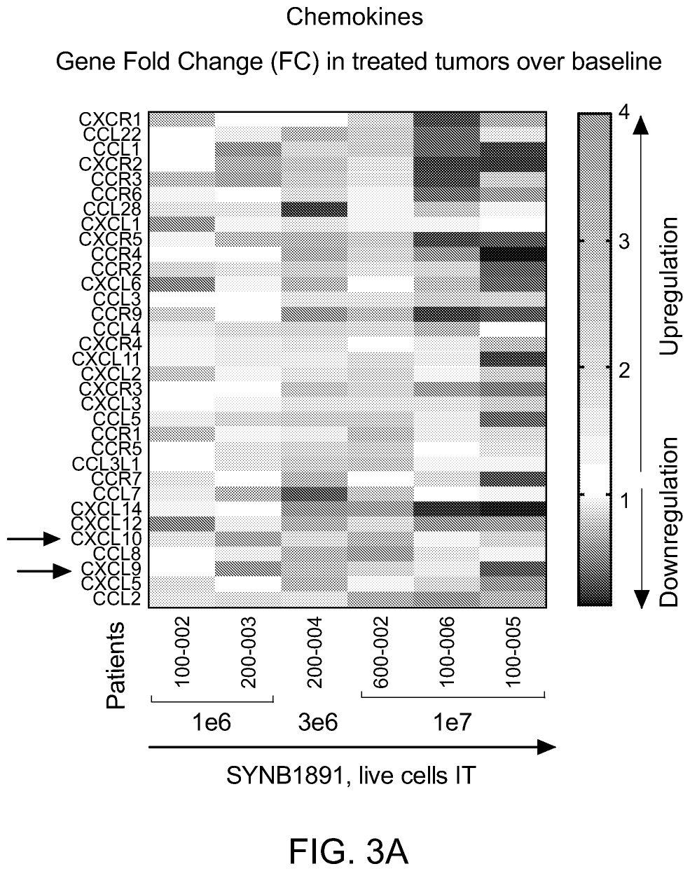

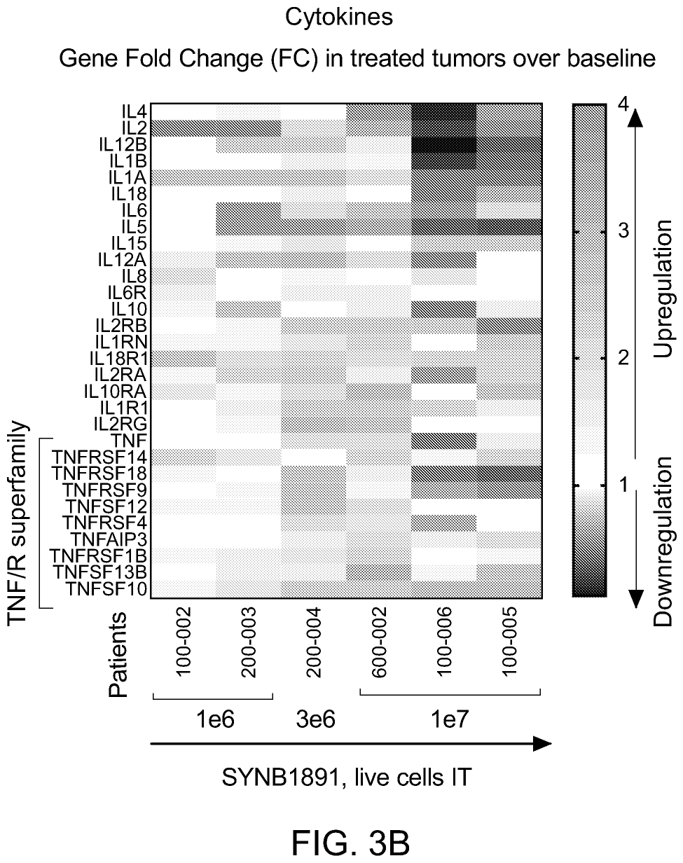

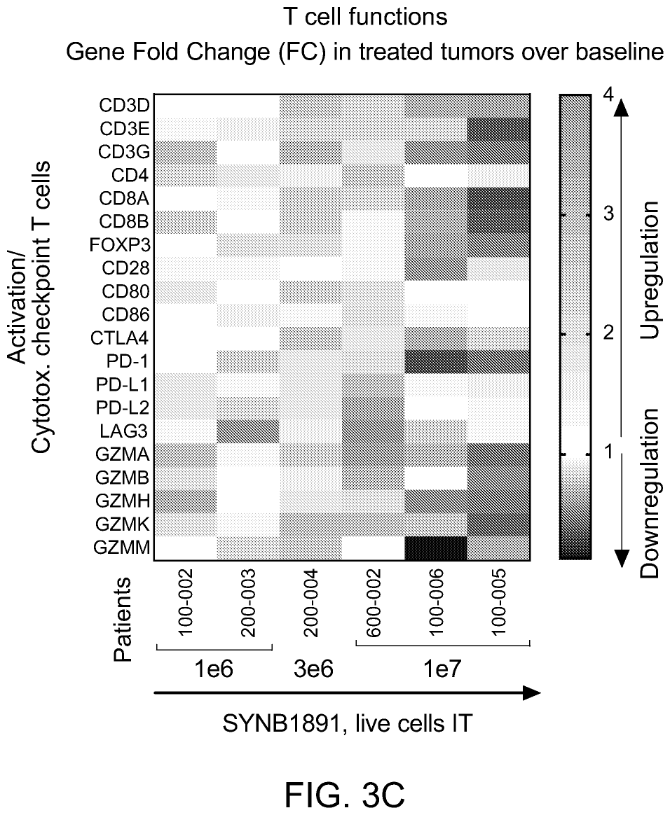

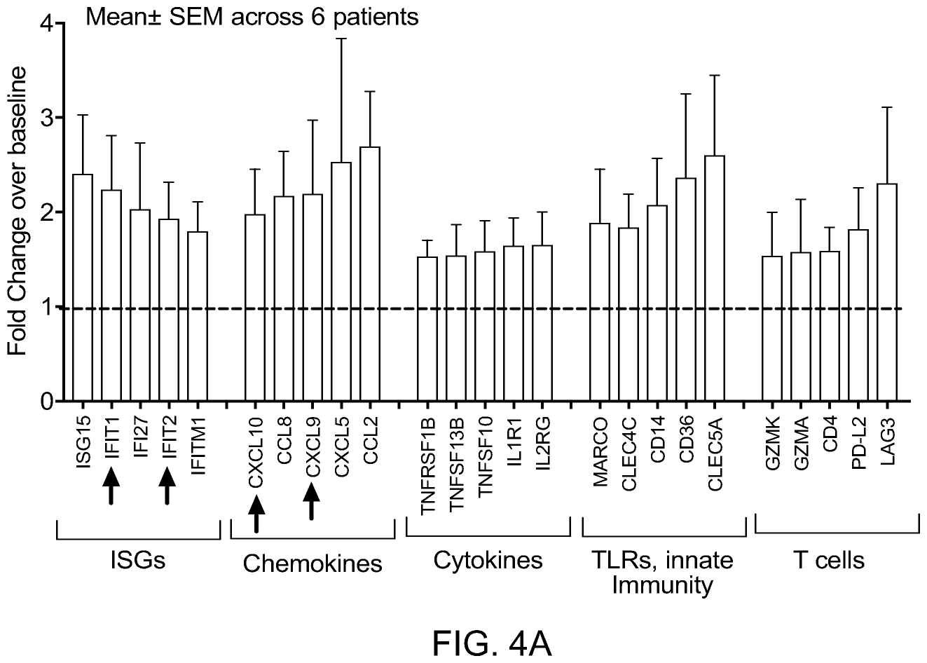

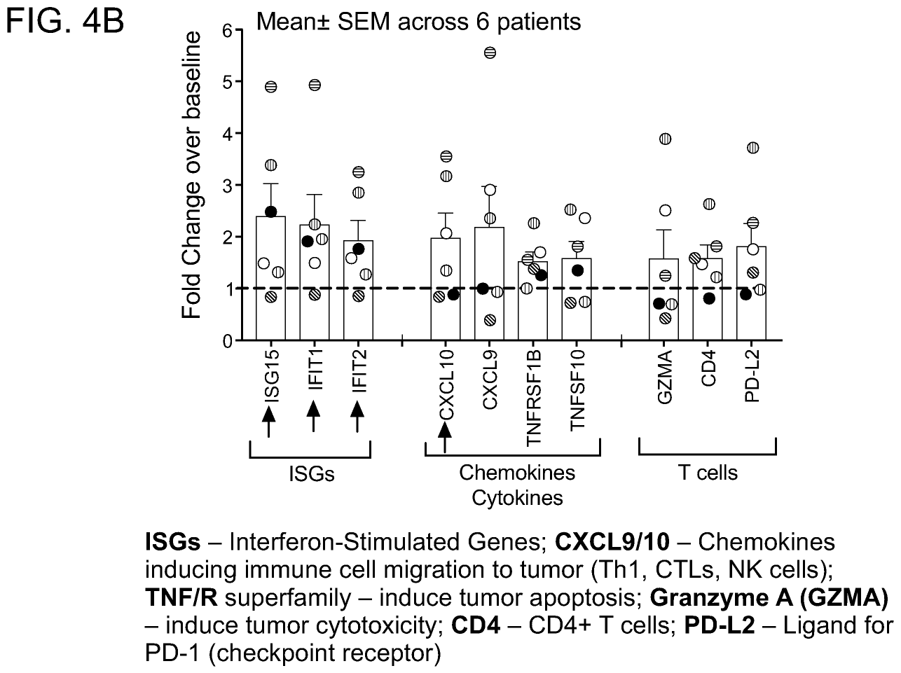

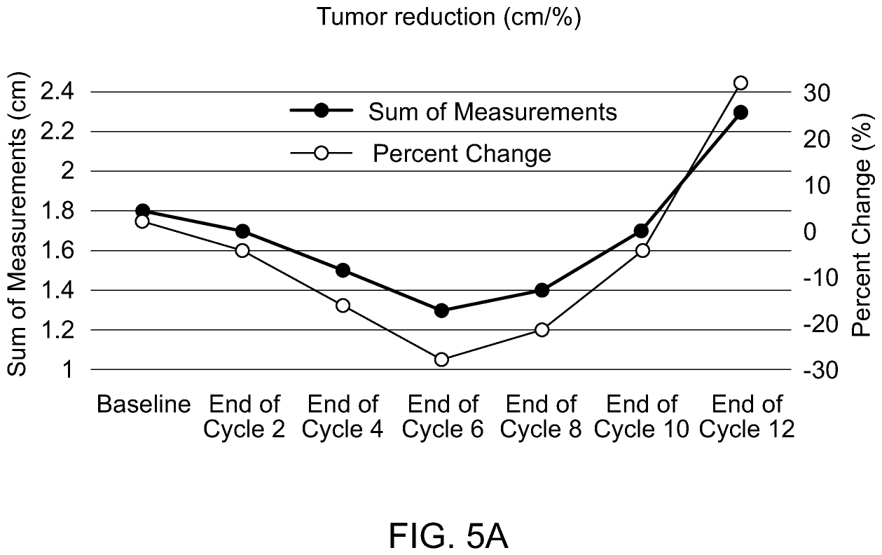

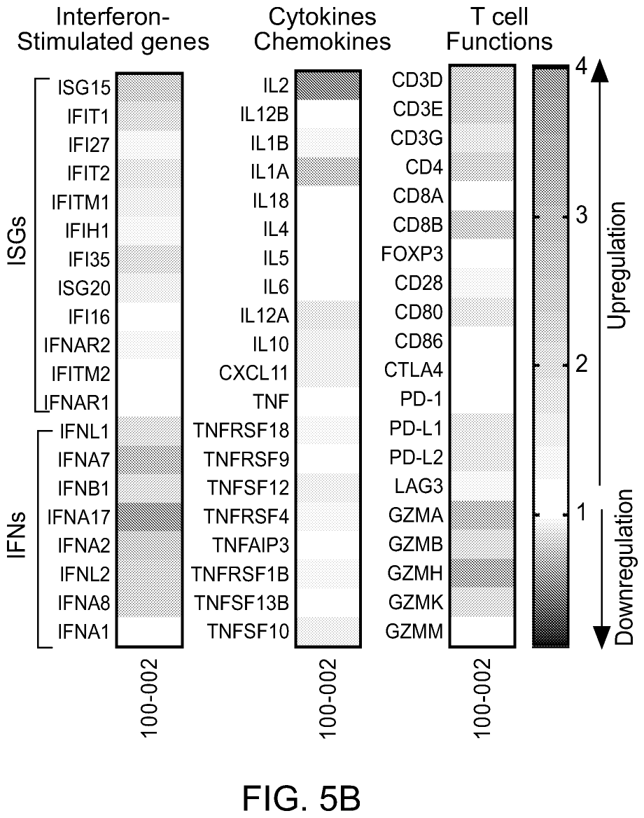

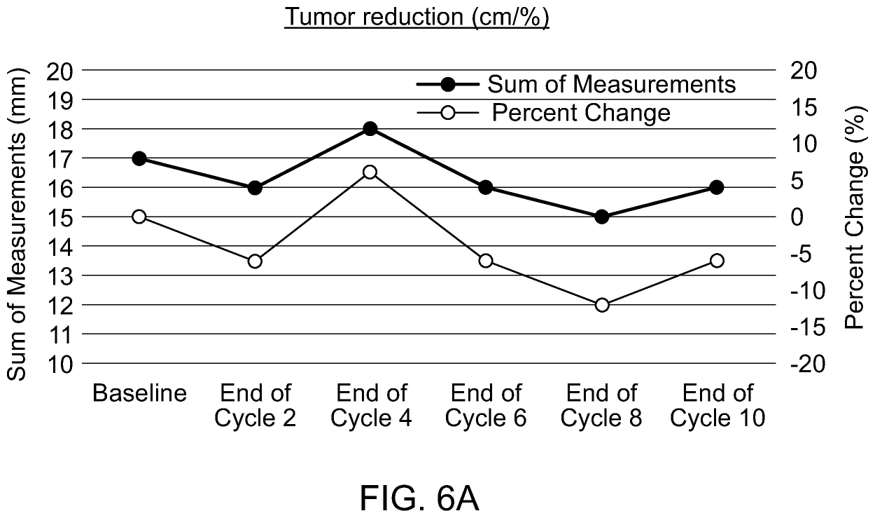

This interim analysis includes the first 11 patients across 4 cohorts dosed at 1×10⁶, 3×10⁶, 1×10⁷, or 3×10⁷ live cells, with a total of 59 doses administered. The mean (range) age was 56 (25-70); 9 patients were female. There were no DLTs, SYNB1891-related infections or discontinuations due to adverse events. There was one SYNB1891-related serious adverse event in a patient who experienced a grade 2 cytokine release syndrome that resolved within one day, and one patient experienced a grade 2 injection site reaction of erythema which resolved. SYNB1891 was not detected in the blood at 6 or 24 hours after the first dose or intratumorally 7 days following the first dose. Treatment with SYNB1891 demonstrated activation of the STING pathway and target engagement as assessed by upregulation of interferon-stimulated genes (ISG15, IFIT1, IFIT2; FIGS. 2A-2B and 4A-4B), chemokines/cytokines (CXCL9, CXCL10, TNFRS1B, TNFSF10; FIGS. 3A-3B) and T-cell response genes (GZMA, CD4, PD-L2; FIGS. 3C and 4A-4B) in core biopsies obtained pre-dose and 7 days following the third weekly dose. CXCL-9/10/11 upregulation which stimulates immune cells migration into tumor: Th1 cells, CTLs, NK cells. In addition, there was a dose-response increase in serum cytokines, IL-6 (FIG. 1A), TNFα (FIG. 1B), IFNγ (FIG. 1C), IL-1Rα (FIG. 1D), at dose levels of 1×10⁷ and 3×10⁷ live cells at 6 hours post-dose. Durable, stable disease was observed in two patients refractory to prior PD-1/L1 antibodies with vulvar melanoma (1×10⁶ live cells; FIGS. 5A-5B) and small cell lung cancer (1×10⁷ live cells; FIGS. 6A-6B).

A 63 year-old female with vulvar melanoma and dosed with 1×10⁶ live cells (FIGS. 5A-5B). The patient previously received local resection and nivolumab. The tumor was genotyped as comprising KIT/PDGFRA/KDR amplification and an ATM deletion.

A 55 year-old female with small cell lung cancer was dosed with 1×10⁷ live cells (FIGS. 6A-6B). The patient previously was treated with etoposide/carboplatin, pegzilarginase, and pembrolizumab.

After approximately 220 days after treatment, the patient with vulvar melanoma and treated with 1×10⁶ live cells and the patient with small cell lung cancer treated with 1×10⁷ live cells had durable, stable disease. Patients with basal cell cancer (treated with 3×10⁶ live cells), squamous cell cancer (treated with 3×10⁷ live cells), bone chondrosarcoma cancer (treated with 1×10⁷ live cells), liposarcoma cancer (treated with 1×10⁶ live cells), sarcoma (breast) cancer (treated with 3×10⁶ live cells), bile duct adeno cancer (treated with 1×10⁷ live cells), esophageal adeno cancer (treated with 1×10⁶ live cells), esophageal cancer (treated with 3×10⁶ live cells), and vulvar squamous cell cancer (treated with 3×10⁷ live cells) were also treated with SYNB1891.

RECIST refers to the Response Evaluation Criteria in Solid Tumors. The patient with vulvar melanoma had 28% RECIST and the patient with small cell lung cancer had 12% RECIST.

Repeat IT injection of SYNB1891 as monotherapy is safe and well-tolerated up to at least 3×10⁷ cells and shows evidence of STING pathway target engagement. These data support the continued dose escalation of SYNB1891 as monotherapy, and initiation of Arm 2 in combination with atezolizumab.

Current enrollment includes 22 patients across 4 sites in the U.S. Patients will be dosed with 1×10⁶ to 1×10⁸ live cells. Patients receiving a combination therapy will receive 1×10⁷ live cells. Tumor types include Tumor types: melanoma [4], sarcoma [4], esophageal [4], squamous (including 2 head and neck) [4], colon/colorectal [2]; small cell lung, basal cell, bile duct adeno, and jejunum adeno.

### Example 2: Immunohistochemistry after Administration of SYNB1891

Multiplex immunofluorescence staining of samples from patients with vulvar melanoma (100-002), liposarcoma (200-003), small cell lung cancer (600-002), and chondrosarcoma of the bone (100-005) showed a change in protein expression in T cells and APC compartment in tumor cells after treatment with SYNB1891. An 8-panel from Ultivue was used to stain for CD4, CD8, Foxp3, Granzyme B, Ki67, PD-L1, MHCII, CD11c (Table 7).

TABLE 7
Markers for Multiplex Immunofluorescence Staining

| Marker | Main Cell Type | Function |
|---|---|---|
| CD4 | Helper T-cell | T-helper cells play an important |
| | | in regulating the immune response. |
| | | The main cell type expressing CD4 is |
| | | mature helper T cells. |
| CD8 | Cytotoxic T cells | Cytotoxic T cells are responsible for |
| | | mediating apoptosis of cancer cells |
| | | through the release of perforin and |
| | | Granzyme B. |
| FoxP3 | Regulatory T cell | Regulatory T cells expressing FoxP3 |
| | | (main Treg transcription factor) |
| | | generally decrease the immune response |
| Granzyme | Cytotoxic T | Granzyme B is produced, stored, and |
| B | cells & NK cells | released by cytotoxic cells to mediate |
| | | cellular death in combination |
| | | with Perforin. |
| Ki67 | Proliferating | Ki67 is expressed within the nucleus |
| | cells | during the phases of mitosis and is |
| | | strongly associated with cell |
| | | proliferation. |
| PD-L1 | Checkpoint | Allows cells to escape immune |
| | protein | surveillance by binding to PD-1. |
| MHC II | Professional | MHC Class II is responsible for the |
| | antigen-presenting | presentation of pathogenic epitopes |
| | cells | to the immune system. |
| CD11c | Dendritic cells | Dendritic cells modulate T cell, B |
| | | cell, monocyte/macrophage function |
| | | to link innate and adaptive immunity |

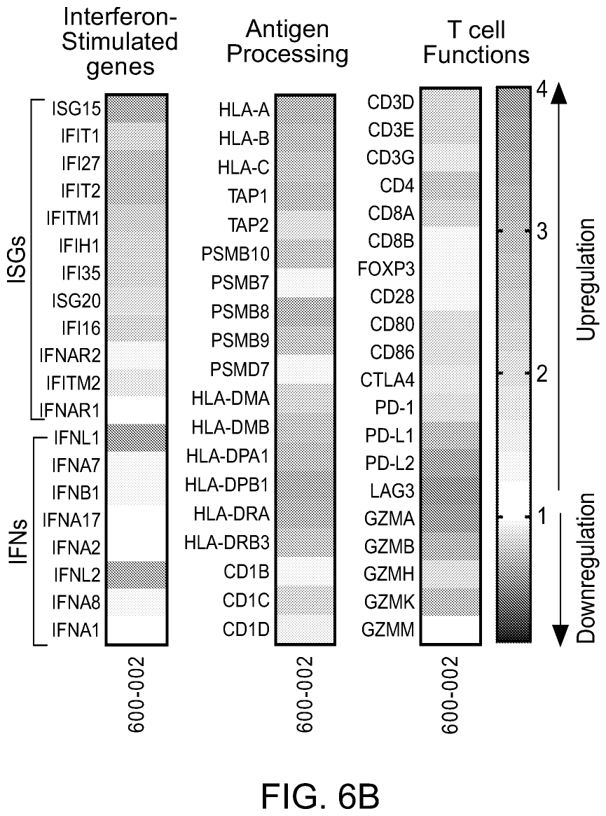

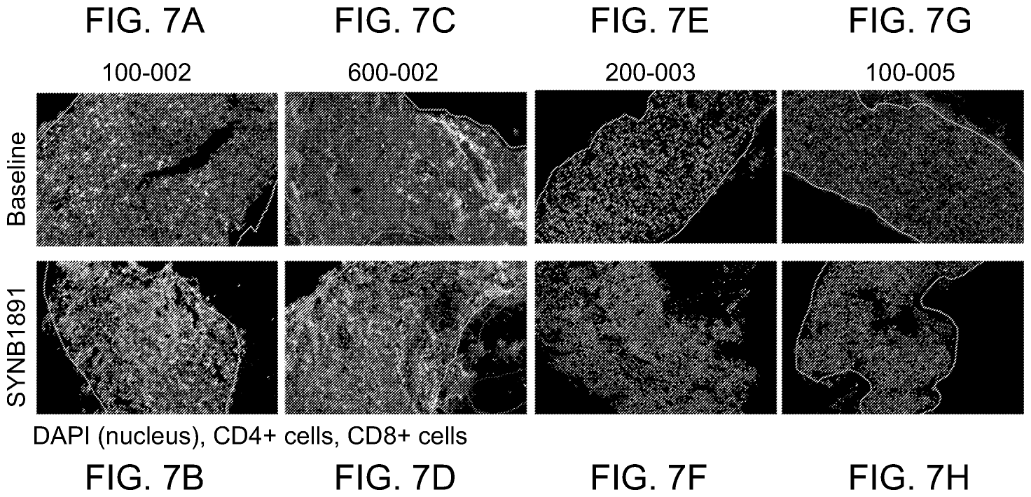

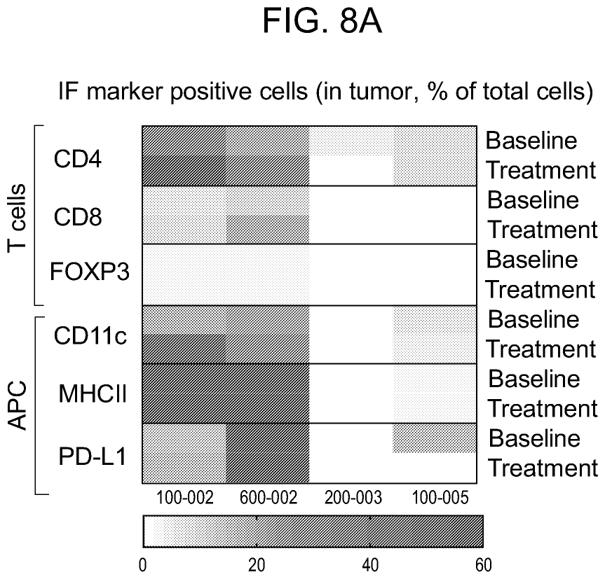

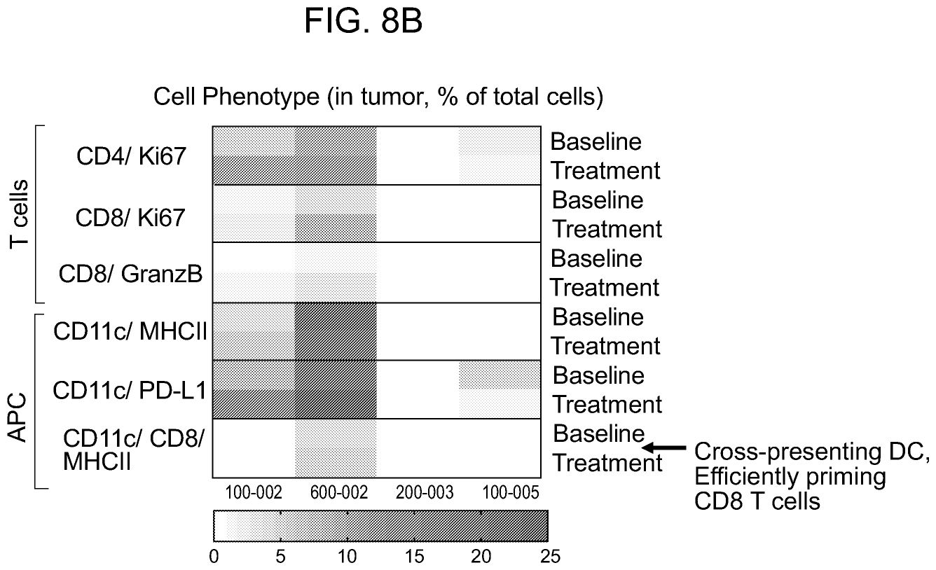

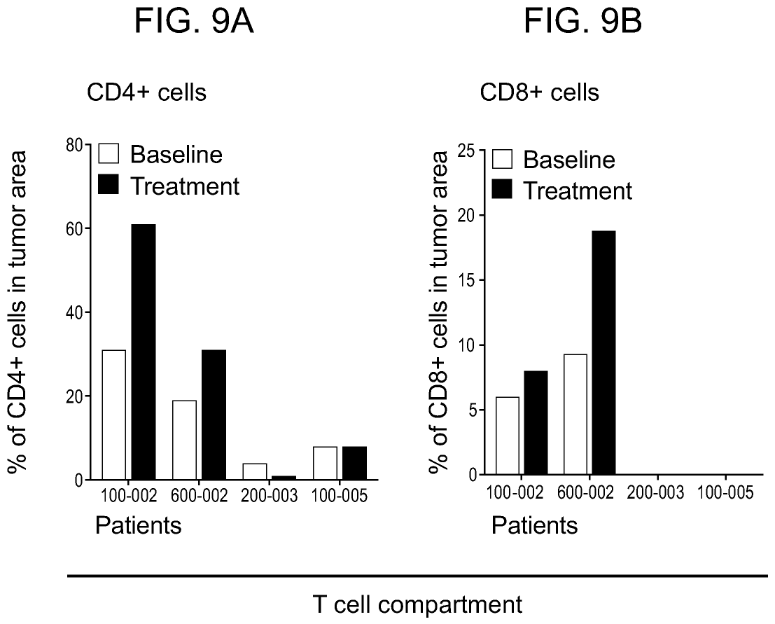

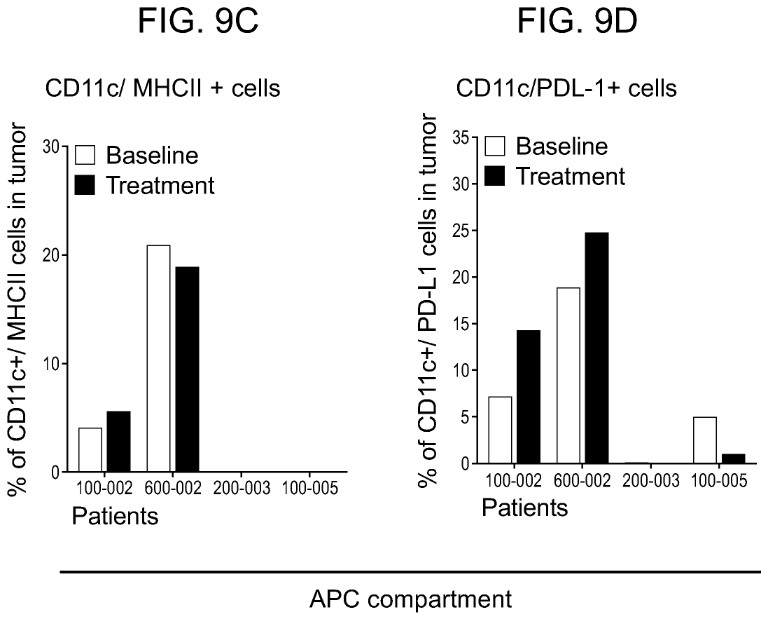

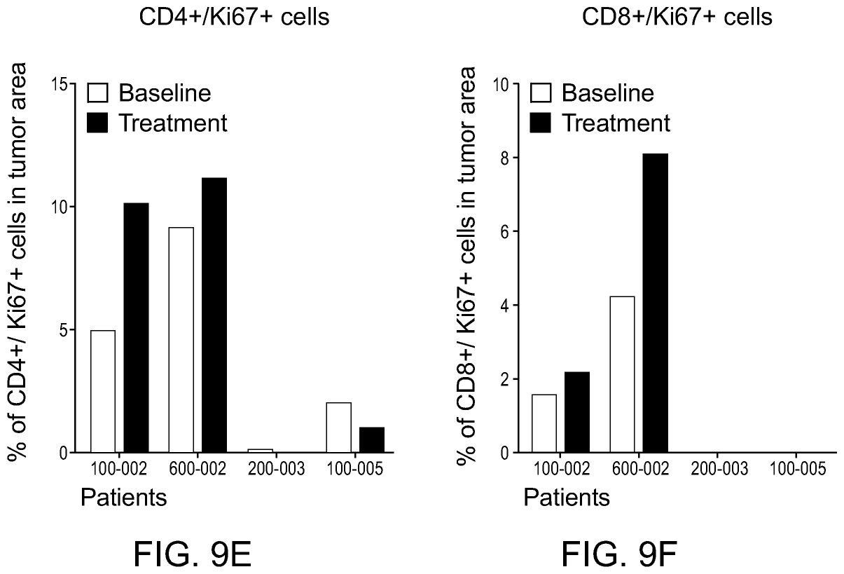

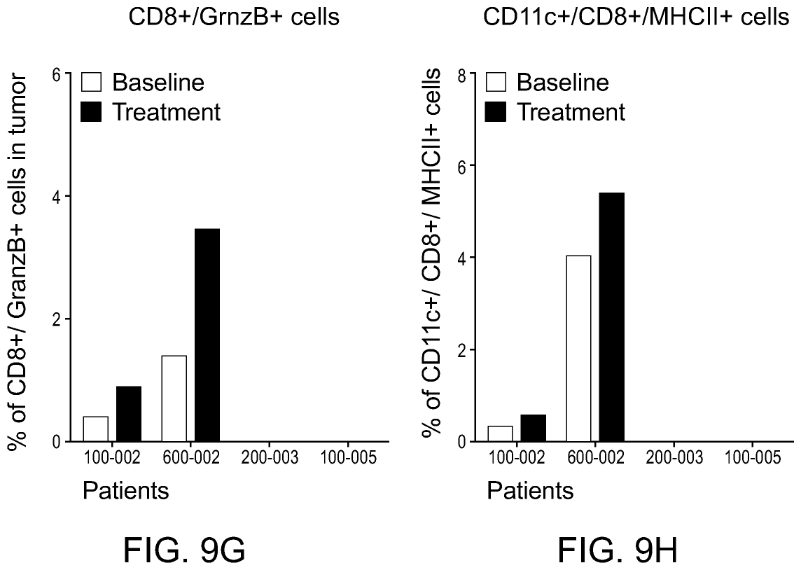

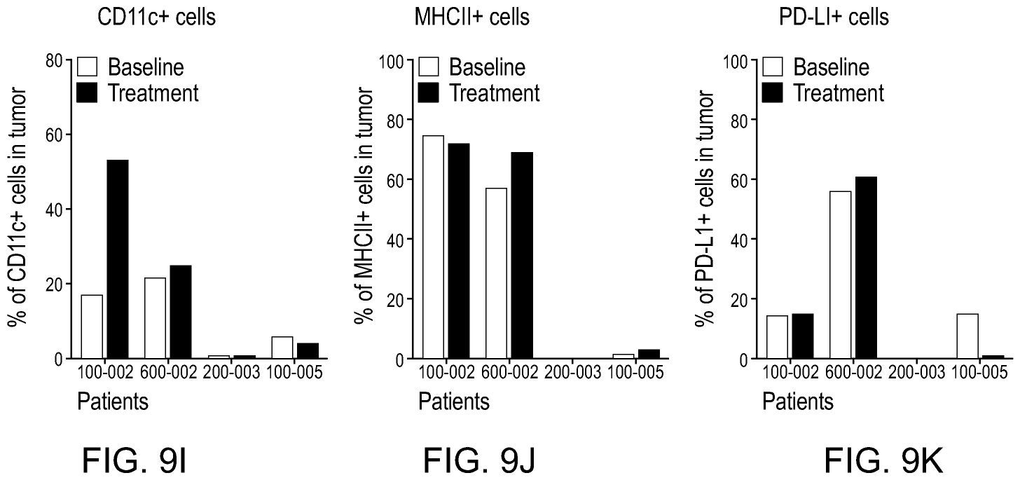

FIGS. 7A-7H show representative images from patients with vulvar melanoma (100-002), liposarcoma (200-003), small cell lung cancer (600-002), and chondrosarcoma of the bone (100-005). Heat map of expression based on multiplex immunofluorescence staining is shown in FIGS. 8A and 8B. Quantification of cells with specific markers are shown in FIGS. 9A-9K. Percent CD4+ cells in a tumor area (FIG. 9A) and percent CD8+ cells in a tumor area (FIG. 9B) represent the T cell compartment. Percent CD11c/MHCII+ cells in a tumor area (FIG. 9C) and percent CD11c/PDL-1+ cells in a tumor area (FIG. 9D) represent the APC compartment. Quantification of percent CD4+/Ki67+ cells (FIG. 9E), CD8+/Ki67+ cells (FIG. 9F), CD8+/GrnzB+ cells (FIG. 9G), CD11c+/CD8+/MHCII+ cells (FIG. 9H), CD11c+ cells (FIG. 9I), MHCII+ cells (FIG. 9J), and PD-LI+ cells (FIG. 9K) in tumor area are shown.

The T cell compartment saw an increase in CD4+ and CD8+ expressing cells in the tumor area after treatment in both patients with vulvar melanoma (100-002) and small cell lung cancer (600-002). The patient with liposarcoma (200-003) saw a decrease of CD4+ expressing cells after treatment, while there was no change over baseline for samples from the patient with chondrosarcoma of the bone (100-005).

The APC compartment was identified by cells expressing CD11c/MHCII or CD11c/PDL-1. The patient with vulvar melanoma (100-002) saw increase in both cell types. The patient with small cell lung cancer (600-002) saw a decrease in CD11c+/MHCII+ cells and an increase in CD11c+/PDL-1+ cells. Neither patient with liposarcoma (200-003) nor chondrosarcoma of the bone (100-005) had cells expressing CD11c/MHCII. The patient with chondrosarcoma of the bone (100-005) saw an increase of CD11c+/PDL-1+ cells, and the patient with liposarcoma (200-003) did not have detectable CD11c+/PDL-1+ cells before or after treatment.

The patient with vulvar melanoma (100-002) saw in increase in CD4+/Ki67+ cells, CD8+/Ki67+ cells, CD8+/GrnzB+ cells, CD11c+/CD8+/MHCII+ cells, CD11c+ cells, MHCII+ cells, PD-LI+ cells after treatment. The patient with small cell lung cancer (600-002) also saw an increase in all cell types except for MHCII+ cells (decreased) and PD-LI+ cells (no change).

### Example 3: Intratumoral Injection of SYNB1891 and Combination with Atezolizumab: Clinical Trial Results

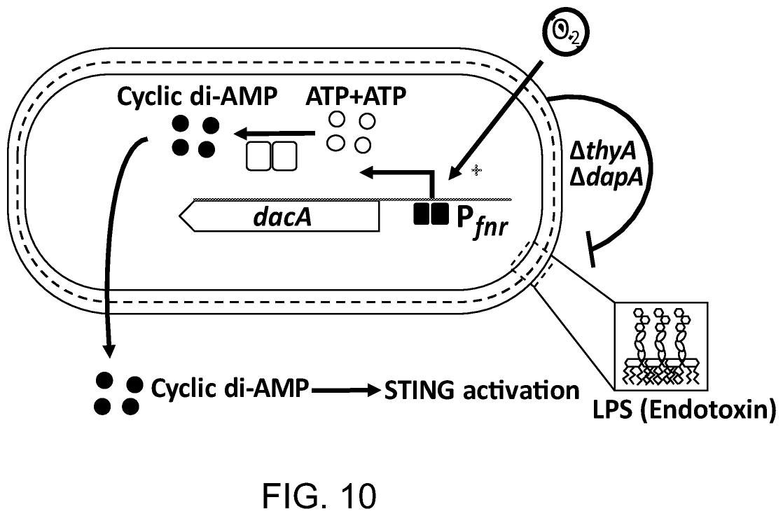

SYNB1891 strain design includes the STING agonist, bacterial chassis, and intra-tumoral delivery. The benefit of the STING agonist it is an innate immune activator, which induces interferon responses and drives T cell priming and recruitment. The bacterial chassis drives immune responses relevant to multiple therapeutic targets. Intra-tumoral delivery provides targeted delivery to antigen presenting cell in the tumor microenvironment. SYNB1891 strain schematic is shown in FIG. 10.

A first-in-human study (NCT04167137; see Example 4 for details) enrolled patients with refractory advanced solid tumors or lymphoma to receive an intratumoral (IT) injection of SYNB1891 either alone or in combination with atezolizumab. Patients enrolled in the monotherapy arms received doses of 1×10⁶-3×10⁸ live cells on Days 1, 8 and 15 of the first 21-day cycle and then on Day 1 of each subsequent cycle. Patients enrolled in the 2 combination cohorts received does of 1×10⁷-3×10⁷ live cells with atezolizumab administered on a 21-day cycle. The primary objective of the study was to evaluate the safety and tolerability of SYNB1891 given alone and in combination of atezolizumab. Other objectives include SYNB1891 kinetics in blood and the injected tumor, STING-target engagement as assessed by IT gene expression and serum cytokines, and tumor responses. This interim analysis includes 24 patients across 6 monotherapy cohorts dosed at 1×10⁶, 3×10⁶, 1×10⁷, or 3×10⁷, 1×10⁸ and 3×10⁸ live cells, and 8 patients dosed in 2 combination therapy cohorts (1×10⁷ and 3×10⁷ live cells). The mean (range) age was 60 (25-82); 20 patients were female. There were 5 cytokine release syndrome events in the monotherapy cohorts, including one which met the criterion for dose limiting toxicity at the 3×10⁸ live cells; there were no other SYNB1891-related serious adverse events. There were no SYNB1891-related infections. SYNB1891 was not detected in the blood at 6 or 24 hours after the first dose or intratumorally 7 days following the first dose. Treatment with SYNB1891 demonstrated activation of the STING pathway and target engagement as assessed by upregulation of interferon-stimulated genes (ISG15, IFIT1, IFIT2), chemokines/cytokines (CXCL9, CXCL10, TNFRS18, TNFSF10) and T-cell response genes (GZMA, CD4, PD-L2) in core biopsies obtained pre-dose and 7 days following the third weekly dose. In addition, there was a dose-response increase in serum cytokines. Durable, stable disease was observed in 3 patients refractory to prior PD-1/L1 antibodies with vulvar melanoma (1×10⁶ live cells), basal cell carcinoma (3×10⁶ live cells) and small cell lung cancer (1×10⁷ live cells). Repeat IT injection of SYNB1891 as monotherapy and in combination atezolizumab in this ongoing study is safe and well-tolerated up to at least 1×10⁸ live cells, and shows evidence of STING pathway target engagement. Specifics of the study are set forth in additional detail below.

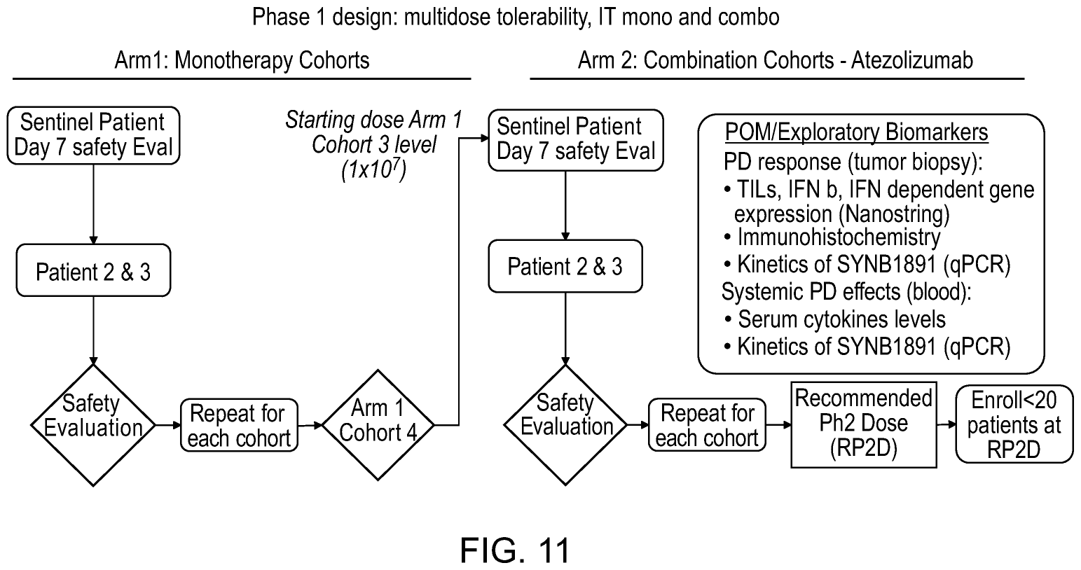

Clinical Phase I design for both multidose tolerability and intra-tumoral injection with and without a combination chemotherapy is shown in FIG. 11. A total of 32 patients received SYNB1891 across 5 sites in the US. 24 patients in Arm 1 (monotherapy) were treated on 6 dose cohorts, 1×10⁶ (n=3), 3×10⁶ (n=3), 1×10⁷ (n=4), 3×10⁷ (n=5), 1×10⁸ (n=3), and 3×10⁸ (n=6) live cells. 8 patients in Arm 2 (combination therapy with standard dose of atezolizumab) were treated on 2 dose cohorts, 1×10⁷ (n=4) and 3×10⁷ (n=4) live cells. 1 patient (Arm 2) remains on treatment and 31 patients discontinued from the study.

29 patients (91%) of the 32 patients were Caucasian, and 3 patients (9%) were Black, with more females (20 patients, 63%) than males (12 patients, 38%) and more patients in the <65 years age category (21 patients, 66%) than ≥65 years category (11 patients, 34%). Tumor types include: Basal cell carcinoma (1), colorectal cancer (1), endometrial cancer (1), esophageal cancer (3), liposarcoma (1), melanoma (7), merkel cell carcinoma (1), NSCLC (1), Other (12), sarcoma (1), SCLC (1), squamous cell carcinoma of skin (1), testicular cancer (1).

153 doses of SYNB1891 (Arms 1 and 2) and 17 doses of atezolizumab (Arm 2) had been administered. Three subjects had durable, stable disease after treatment with SYNB1891: 100-002 vulvar melanoma (228 days; 1×10⁶ live cells), 600-002 small cell lung cancer (>342 days; 1×10⁷ live cells) and 100-004 basal cell carcinoma (83 days; 3×10⁶ live cells).

Fourteen patients (44%) experienced TEASs related to SYNB1891. In Arm 2, no adverse events (AEs) were assessed as related to atezolizumab treatment. Four patients (13%) experienced serious adverse events (SAEs) that were considered related to SYNB1891 treatment, all of which were events of cytokine release syndrome (CRS).

Five patients (16%), all in Arm 1, experienced cytokine release syndrome (CRS) with 2 patients experiencing CRS after the first dose of SYNB1891 and 3 patients after the second dose. One CRS event, occurring at the dose of 3×10⁸ live cells, was assessed as grade 3, was associated with respiratory failure and met the criteria for dose-limiting toxicity (DLT). Three of the 5 patients were successfully able to continue treatment; 1 patient came off study due to CRS and another patient stopped treatment for an unrelated reason.

Four patients (13%) experienced injection site reactions. These reactions were variably treated with supportive medications and prophylactic medications on subsequent cycles. Injection site reactions did not prohibit further dosing with SYNB1891.

No systemic infections were considered related to SYNB1891.

Dose-limiting toxicity (DLT) was observed for 1 patient (grade 3 CRS) at the highest administered dose level in Arm 1 (3×10⁸ live cells). No DLTs were observed in Arm 2. No maximum tolerated dose/recommended phase II dose (MTD/RP2D) was determined for either Arm 1 or Arm 2 prior to the study being terminated. The maximum administered doses of SYNB1891 were deemed safe in both arms.

Table 8 outlines the biomarker schedule during the study period. Analysis of panel of top cytokine markers from blood draws in preclinical studies (IL-1RA, IFN-γ, IL-1β, IL-2, IL-4, IL-6, IL-8, IL-10, IL-12p70, IL-13, TNF-α) was performed. SYNB1891 kinetics was studied for SYNB1891 quantification in blood draws by qPCR (LLOQ is 25 DNA copies/mL). Tumor fine needle aspirate was studied for SYNB1891 quantification in the tumor (LLOQ is 25 DNA copies/g). Biopsy of eligible lesion were performed by formalin-fixed paraffin-embedded (FFPE) processed for pathology and NanoString tumor immune infiltrate analysis.

TABLE 8
SYNB1891 PK and BioMarker Schedule

| | Cycle 1 | | | | | | | | |
|---|---|---|---|---|---|---|---|---|---|
| | 1 | | | 2 | 3 | | | Cycles | Additional |
| | | | (6 ± 1 | (24 ± 2 | (48 ± 2 | | | 2-4 | Cycles |
| Study Period | Baseline | | hours | hours | hours | 8 ± | 15 ± | 1 ± | 1 ± |
| Study Day | (predose) | Dose | postdose) | postdose) | postdose) | 1 day | 1 day | 1 day | 1 day |
| Serum Cytokines | ● | | ● | ● | | ● | ● | ● (a) | ● (a) |
| Kinetics of Blood | ● | | ● | ● | | | | | |
| SYNB1891 | | | | | | | | | |
| (qPCR assay) | | | | | | | | | |
| Tumor Fine | ● | | | | | ● | | | |
| Needle Aspirate | | | | | | | | | |
| (SYNB PK) | | | | | | | | | |
| Biopsy of | ● | | | | | | | ● (b) | |
| eligible lesion | | | | | | | | | |
| Biopsy of non- | ● | | | | | | | ● (b) | |
| injected lesion (if | | | | | | | | | |
| completed) | | | | | | | | | |

(a) Day 1 (pre-dose and +6 hours) of Cycles 2, 3 and 4 (or additional cycles)
(b) Cycle 2 Day 1 only

Table 9 outlines NanoString Set.

TABLE 9

| | | | | Max | | |
|---|---|---|---|---|---|---|
| | | | | Change | | |
| | | | Baseline | from | | Treatment duration, |
| Patient | Tumor Type | Dose | (injected) | Baseline | | Weeks/# of doses |
| 100-002 | Vulvar melanoma | 1e6 | 18 mm | 13 | mm | 33 weeks/14 doses (Cycle 12) |
| 200-003 | Liposarcoma | 1e6 | 145 mm | 179 | mm | 3 weeks/4 doses (Cycle 2) |
| 200-004 | Sarcoma (left | 3e6 | 52 mm | 90 | mm | 3 weeks/4 doses (Cycle 2) |
| | chest, breast) | | | | | |
| 600-002 | Right neck mass, | 1e7 | 17 mm | 15 | mm | 45 weeks/18 doses (Cycle 16) |
| | SCLC | | | | | |
| 100-005 | Chondrosarcoma | 1e7 | 95 mm | 104 | mm | 3 weeks/4 doses (Cycle 2) |
| | of the bone | | | | | |
| 100-006 | Cutaneous | 1e7 | 57 mm | 57 | mm | 6 weeks/5 doses (Cycle 3) |
| | squamous cell | | | | | |
| | carcinoma | | | | | |
| 500-001 | Melanoma | 3e7 | 10 mm | Not | | 3 weeks/4 doses (Cycle 2) |
| | | | | done | | |
| 100-010 | Oropharynx | 1e8 | 22 mm | 23 | mm | 24 weeks/11 doses (Cycle 9) |
| | Squamous Cell | | | | | |
| | Carcinoma | | | | | |
| 600-006 | Melanoma | 1e8 | 49 mm | 48.9 | mm | 9 weeks/6 doses (Cycle 4) |
| 500-003 | Melanoma | 3e8 | 22 mm | 34 | mm | 3 weeks/4 doses (Cycle 2) |
| 100-014 | Metastatic | le7 + | 22 mm | 23 | mm | 6 weeks/5 doses (Cycle 3) |
| | adenocarcinoma of | aPD1 | | | | |
| | jejunum | | | | | |
| 600-007 | Esophageal | le7 + | 30 mm | 35.6 | mm | 6 weeks/5 doses (Cycle 3) |

| | |
|---|---|
| cancer | aPD1 |

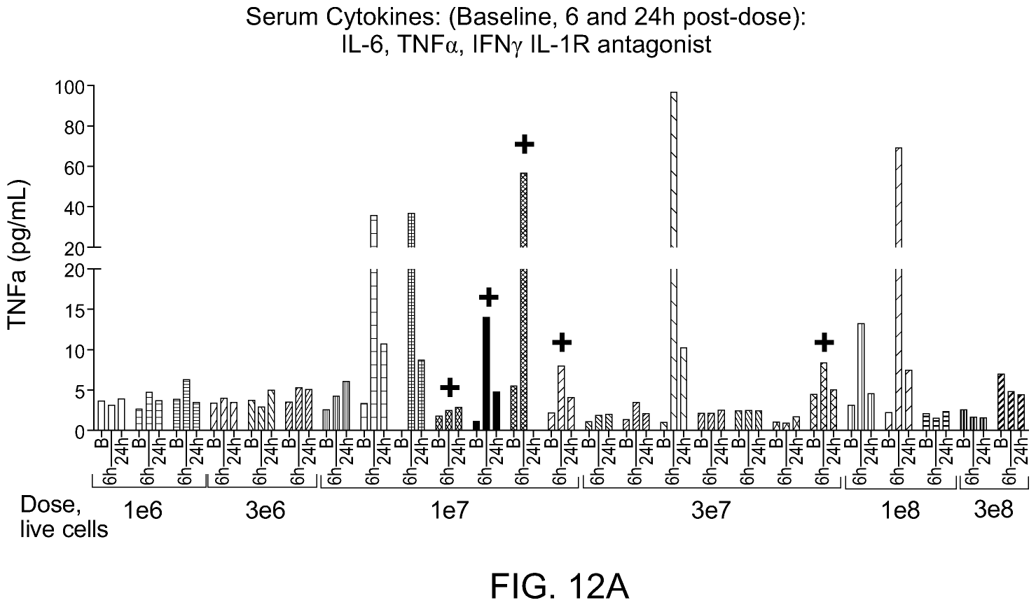

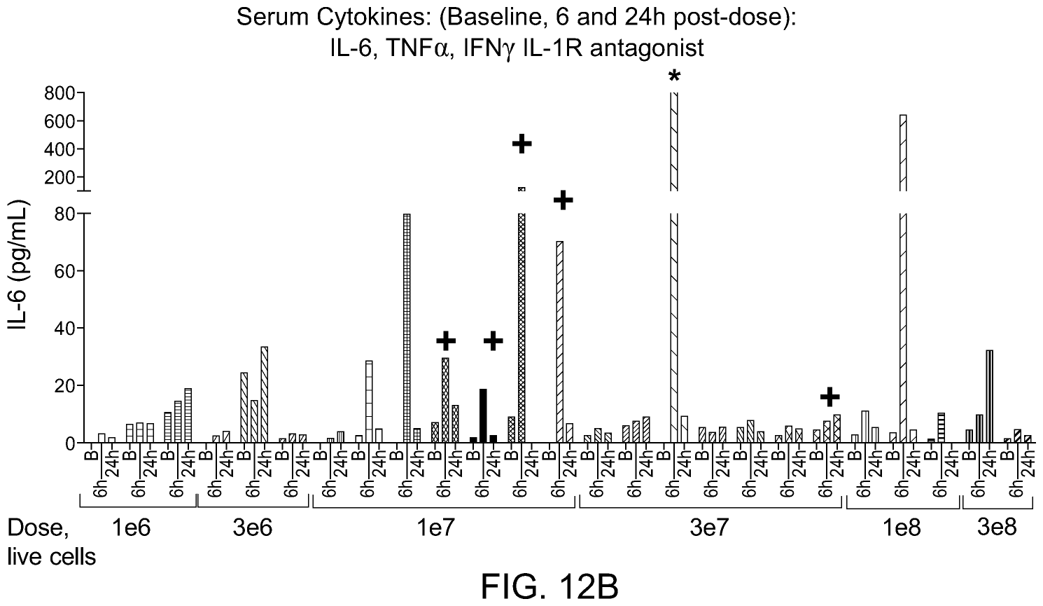

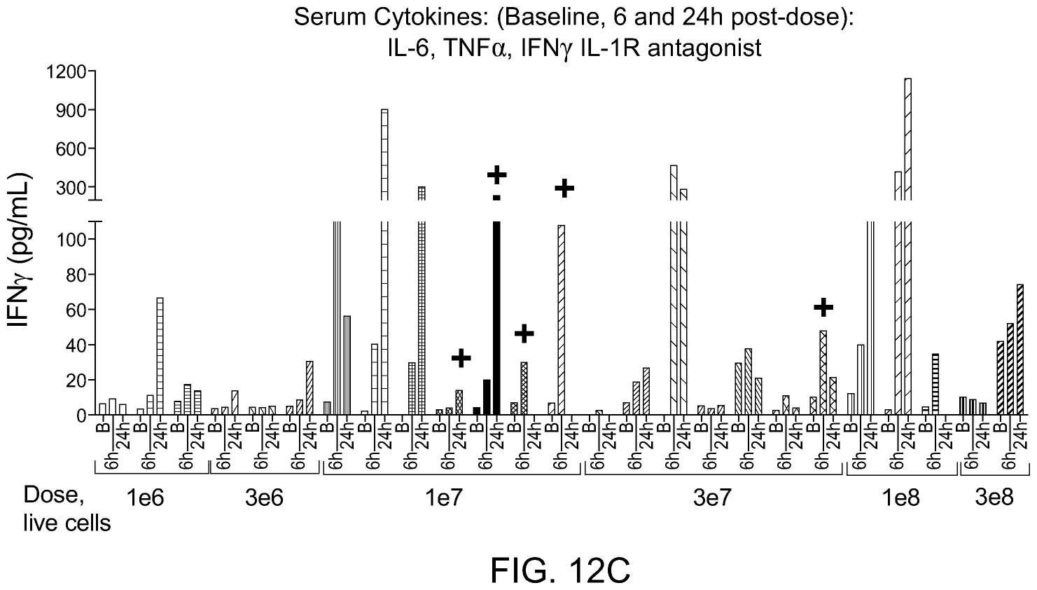

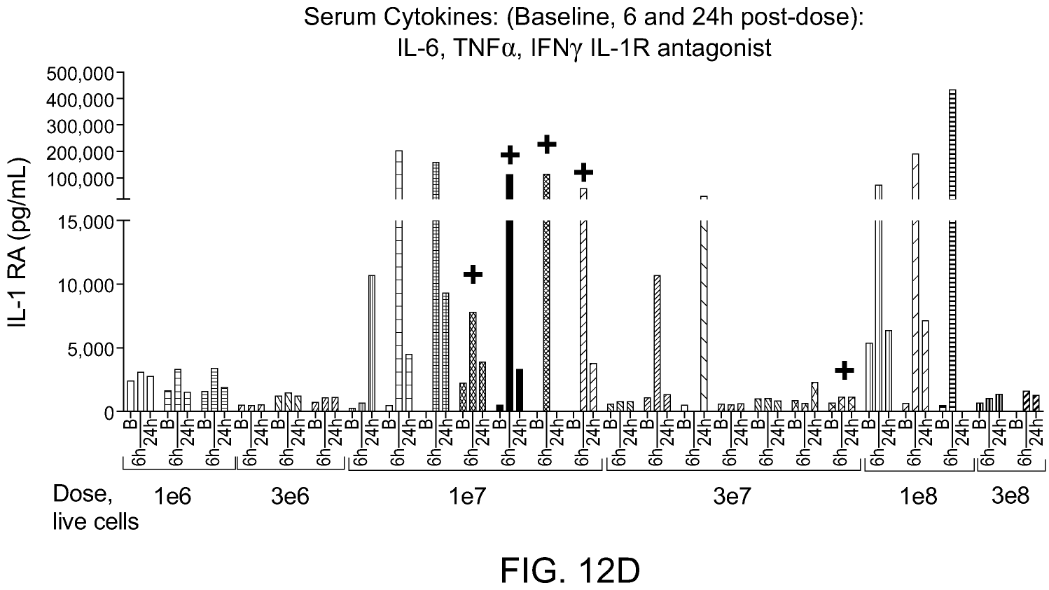

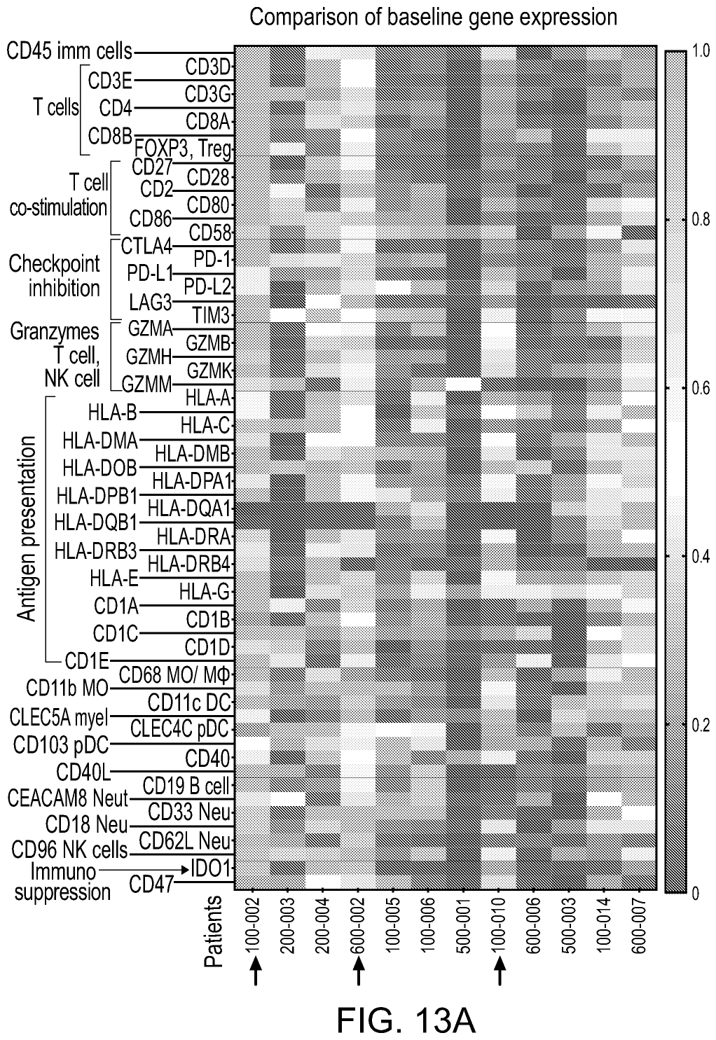

FIGS. 12A-12D depict serum cytokines at baseline, 6 hours and 24 hours after injection of 1×10⁶, 3×10⁶, 1×10⁷, 3×10⁷, 1×10⁸, and 3×10⁸ live cells. Cytokines include IL-6 (FIG. 12A), TNFα (FIG. 12B), IFNγ (FIG. 12C), and IL-1RA (FIG. 12D).

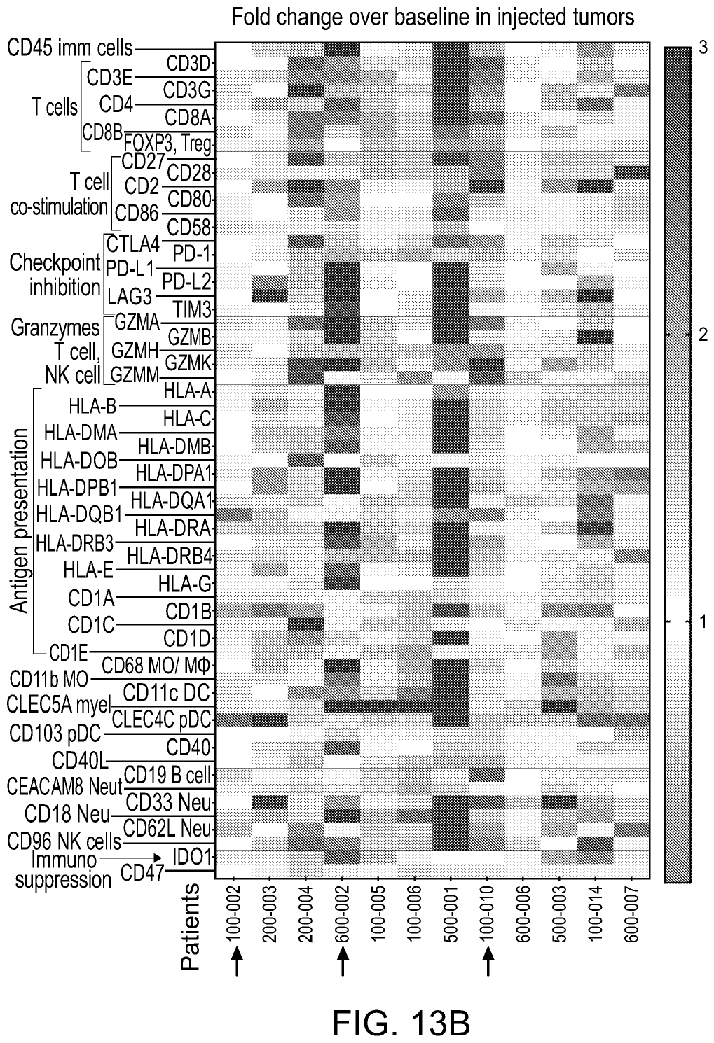

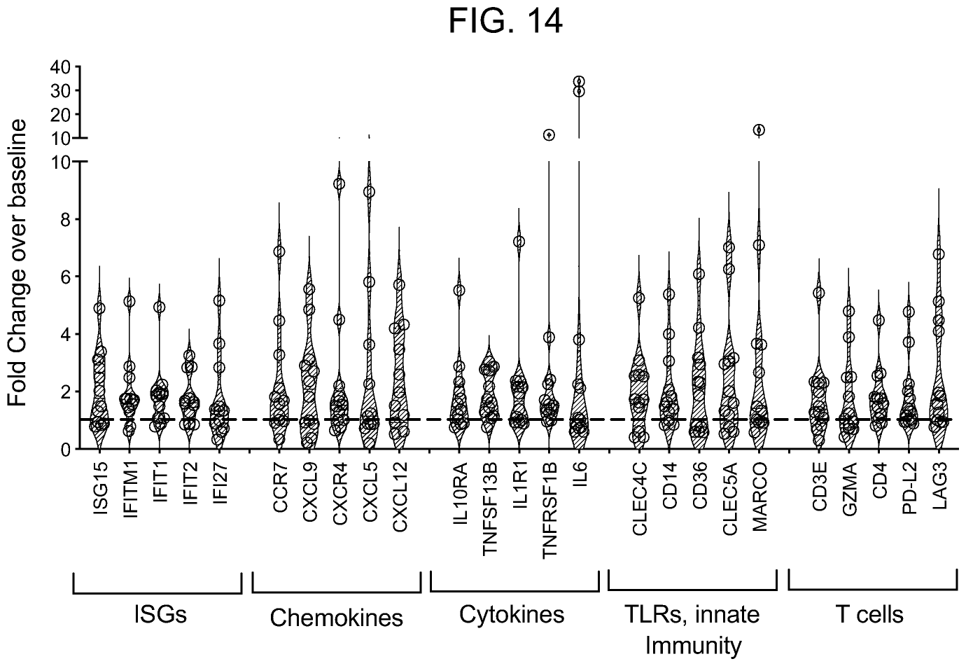

Baseline gene expression levels across different tumor types is depicted in FIG. 13A. Data were normalized to the highest and lowest expressed gene in each row. The majority of injected tumors had low levels of inflammatory gene expression. Fold change in gene expression over baseline in injected tumors (FIG. 13B). Fold changes in gene expression after 1 cycle of SYNB1819 treatment relative to baseline samples as determined by Nanostring. Data were available for the following tumor types (live cell dose): 100-002 Vulvar melanoma (1e6), 200-003 Liposarcoma (1e6), 200-004 Sarcoma (left chest, breast; 3e6), 600-002 SCLC (1e7), 100-005 Chondrosarcoma (1e7), 100-006 Cutaneous squamous cell carcinoma (1e7), 500-001 Melanoma (3e7), 100-010 Oropharynx Squamous Cell Carcinoma (1e8), 600-006 Melanoma (1e8), 500-003 Melanoma (3e8), 100-014 Metastatic adenocarcinoma of jejunum (1e7+aPD1), 600-007 Esophageal cancer (1e7+aPD1). Subjects 100-002 and 600-002 had durable, stable disease.

Change from baseline in gene expression for the 5 most upregulated genes across 5 different gene categories is shown in FIG. 14. Data are shown for the same 12 patients depicted in FIG. 13A and FIG. 13B. Each symbol depicts the data for a single patient.

In conclusion, repeat IT injection of SYNB1891 is safe and well-tolerated in a heterogeneous population up to a dose of 3×10⁸ live cells. No infections related to SYNB1819 were seen, and SYNB1819 demonstrated target engagement as assessed by increases in serum cytokines and upregulation of interferon stimulated genes (ISGs). Evidence of durable stable disease was seen in 3 patients, and was associated with upregulation of genes tied to immune activation lymphocytes in 2 of the subjects. Gene expression data was not available for the third subject with durable, stable disease.

### Example 4: Clinical Protocol for Intratumoral Injection of SYNB1891 and Combination with Atezolizumab

The protocol for the first-in-human clinical trials referenced in the Examples above is provided in Example 4.

### Study Dosing Regimens: Treatments Administered

Patients enrolled to Arm 1 may receive up to four 21-day cycles of SYNB1891 monotherapy. On Days 1, 8, and 15 of Cycle 1 and Day 1 of Cycles 2 through 4, patients receive an i.t. injection of SYNB1891 into an eligible lesion. If the initial eligible lesion undergoes complete regression and is no longer injectable (at the discretion of the Investigator), a subsequent eligible lesion (until no more eligible lesions remain) may be injected.

At the end of Cycle 4, patients in Arm 1 who do not have progressive disease (i.e., those who achieve and sustain complete response [CR], partial response [PR], or stable disease [SD]) may receive additional cycles of SYNB1891 administered by i.t. injection on Day 1 of each cycle for up to 24 months (i.e., Cycles 5 to 35) after the initial dose of study treatment until documentation of progressive disease or other discontinuation criteria, satisfaction of a predefined study stopping rule, or no eligible lesions remain.

Patients enrolled to Arm 2 may receive up to four 21-day cycles with SYNB1891 administered by i.t. injection into an eligible lesion on Days 1, 8, and 15 of Cycle 1 and Day 1 of Cycles 2 through 4. In addition, atezolizumab will be administered in accordance with its recommended dose and schedule (1200 mg IV Q3W) on Day 1 of each of the 4 planned cycles. On days when atezolizumab and SYNB1891 are both administered, SYNB1891 will be administered first, followed by at least 1 hour of observation prior to the atezolizumab infusion. Patients in Arm 2 who do not have progressive disease (i.e., those who achieve and sustain CR, PR, or SD) at the end of Cycle 4 on combination therapy may receive additional cycles of SYNB1891 and atezolizumab for up to 24 months (i.e., Cycles 5 to 35) after the initial dose of study treatment until documentation of progressive disease or other discontinuation criteria, satisfaction of a predefined study stopping rule, or no eligible lesions remain.

### Postdose Monitoring

The initial patient in each Arm 1 cohort (Sentinel patient) will be monitored for at least 6 hours following the first (Cycle 1 Day 1) i.t. injection of SYNB1891 (or overnight, at the discretion of the Investigator) for evaluation of any procedural complication or acute toxicity (i.e., injection reactions or cytokine release syndrome) and for collection of postdose blood samples. Safety data must be reviewed on or after Day 7 and deemed acceptable by the Investigator and the Sponsor and contract research organization (CRO) Medical Monitors before the Sentinel patient receives any further injections and before any subsequent patients are dosed in that cohort.

For the remainder of Cycle 1 dosing for the Sentinel patient and for all dosing for non-Sentinel patients, SYNB1891 injections will be administered in the outpatient setting and patients will be closely observed at the clinic for at least 6 hours following each injection for evaluation of any procedural complication or acute toxicity (i.e., injection reactions or cytokine release syndrome) and for collection of postdose blood samples. All patients will be contacted by telephone on Cycle 1 Day 3 to monitor for signs of cytokine release syndrome.

Dose-Finding with the mTPI Algorithm

In both study arms, each cohort will initially comprise 3 patients (a Sentinel patient and 2 non-Sentinel patients), with subsequent dose escalation governed by an mTPI design (Ji et al 2013).

The starting dose of SYNB1891 in the first cohort of Arm 1 will be 1×10⁶ live cells and will be increased in approximately 3-fold increments in subsequent cohorts until MTD determination. If no MTD is identified, then the MTD will be considered the maximum dose at which a sufficient number of patients have been treated such that if 14 patients were administered that dose then either escalation or staying at the current dose would be recommended according to Table 1. For example, if 8 patients were administered a dose level with 0 DLTs observed, even if all remaining 6 patients had a DLT, Table 10 would indicate “stay at the current dose”; thus, the remaining 6 patients are not required to evaluate the MTD.

A mTPI design with a target DLT rate of approximately 30% will be applied for dose escalation and confirmation to determine the MTD/RP2D. In Arm 1, predetermined dose levels of 1×10⁶, 3×10⁶, 1×10⁷, 3×10⁷, 1×10⁸, 3×10⁸, and 1×10⁹ live cells will be explored independently, with additional dose levels as necessary to determine the MTD. A de-escalation dose of 3×10⁵ live cells is available if the starting dose is deemed not tolerable. All dose escalation and de-escalation decisions will be based on the occurrence of DLTs at a given dose during Cycle 1 and will be made jointly by the Investigators and the Sponsor.

In Table 10, the number of patients treated is indicated in the columns and the number of patients who experienced a DLT is indicated in the rows. Dosing decisions include escalate to the next higher dose (E), stay at the current dose (S), de-escalate to the next lower dose (D), and de-escalate to a lower dose and never test this dose again (i.e., unacceptably toxic dose; DU). During dose escalation, a minimum of 3 patients are required at each dose. Depending on accrual rate and occurrence of DLTs, 3, 4, 5 or 6 patients may be enrolled at each new dose until the last of those patients completes the Cycle 1 DLT assessment period. For example, the dose escalation rules will proceed as follows if 3 patients are enrolled: if 0 of the first 3 patients at a given dose level develops a DLT, then the dose can be escalated to the next level without further expansion. If 1 of the first 3 patients at a given dose level develops a DLT, no more than an additional 3 patients should be enrolled at this dose level until additional DLT data are available since this dose would be considered unacceptably toxic if all 3 of the additional patients experience a DLT (i.e., 4 of 6 patients). If 2 of the first 3 patients at a given dose level develop a DLT, the dose will be de-escalated to the next lower level. If 3 of the first 3 patients at a given dose level develop a DLT, this dose will be considered unacceptably toxic (i.e., the dose will be de-escalated and never re-escalated to that dose again). The same principle will be applied whether 3, 4, 5 or 6 patients are enrolled in the same dose cohort according to Table 10.

Based on the mTPI design, the number of patients who are enrolled at a dose, but are not yet fully evaluable for DLT assessment, may not exceed the number of remaining patients who are at risk of developing a DLT before the dose would be considered unacceptably toxic (denoted as DU in Table 10). To determine how many more patients can be enrolled at a dose level, one can count steps in a diagonal direction (down and to the right) from the current cell to the first cell marked DU. In total, 3 to 14 patients may be enrolled at a given dose level.

Dose escalation and confirmation will end after 14 patients have been treated at any of the selected doses. The pool-adjacent-violators-algorithm (Ji et al 2013) will be used to estimate the DLT rates across doses. The dose with an estimated DLT rate closest to 30% may be treated as a preliminary MTD. Using the mTPI algorithm, the totality of the data will be considered before a dose is selected to carry forward to the next cohort, and the escalation schedule may be adjusted based on PK, PD, and safety data emerging throughout the study to determine the MTD/RP2D. Note that while 30% was the target toxicity rate used to generate the guidelines in Table 10, the observed rates of patients with DLTs at the MTD may be slightly above or below 30%.

TABLE 10
Dose-finding Rules per mTPI Design

| Number of participants | Number of Participants Evaluable for DLT at Current Dose | | | | | | | | | | | |
|---|---|---|---|---|---|---|---|---|---|---|---|---|
| with at least 1 DLT | 3 | 4 | 5 | 6 | 7 | 8 | 9 | 10 | 11 | 12 | 13 | 14 |
| 0 | E | E | E | E | E | E | E | E | E | E | E | E |
| 1 | S | S | S | E | E | E | E | E | E | E | E | E |
| 2 | D | S | S | S | S | S | S | S | E | E | E | E |
| 3 | DU | DU | D | S | S | S | S | S | S | S | S | S |
| 4 | | DU | DU | DU | D | D | S | S | S | S | S | S |
| 5 | | | DU | DU | DU | DU | DU | D | S | S | S | S |
| 6 | | | | DU | DU | DU | DU | DU | DU | D | S | S |
| 7 | | | | | DU | DU | DU | DU | DU | DU | DU | D |
| 8 | | | | | | DU | DU | DU | DU | DU | DU | DU |
| 9 | | | | | | | DU | DU | DU | DU | DU | DU |
| 10 | | | | | | | | DU | DU | DU | DU | DU |
| 11 | | | | | | | | | DU | DU | DU | DU |
| 12 | | | | | | | | | | DU | DU | DU |
| 13 | | | | | | | | | | | DU | DU |
| 14 | | | | | | | | | | | | DU |

E = escalate to the next higher dose; S = stay at the current dose; D = de-escalate to the next lower dose; DU = the current dose is unacceptably toxic.
Target toxicity rate = 30%.
Flat noninformative prior Beta (1,1) is used as a prior and ε1 = ε2 = 0.03.

After all patients in Arm 1 Cohort 4 have completed their Cycle 1 DLT safety evaluation, dosing will begin in Arm 2 starting at the Arm 1 Cohort 3 dose level. Combination doses will not be escalated above the SYNB1891 single-agent MTD established in Arm 1. Once dosing in Arm 2 is initiated, patient allocation to cohorts will be managed by the Sponsor to ensure that a dosing level within Arm 2 is at least one dose level below the SYNB1891 monotherapy dose being evaluated in Arm 1. The atezolizumab dose will remain constant at 1200 mg Q3W for each dose level in Arm 2.

In both study arms, enrollment will proceed in accordance with the following rules: Review of the safety data for the first patient in a cohort (Sentinel patient) must be completed on or after Day 7 before the remaining 2 patients (non-Sentinel) in that cohort may be dosed (these 2 patients may be dosed concurrently). Observation of DLT(s) in a Sentinel patient will not necessarily preclude enrollment of subsequent patients at the same dose level; rather, DLTs occurring in Sentinel patients will be reviewed individually by the SRC (consisting of the Sponsor Medical Monitor, the CRO Medical Monitor, all active Investigators, and other study personnel as needed) to determine appropriate dosing for subsequent patients.

After all patients in a cohort have been assessed for DLTs (i.e., after each patient either experiences a DLT or completes the end of Cycle 1 safety assessment, whichever occurs first), the SRC will review all available safety data from the cohort and determine whether to escalate to the next higher SYNB1891 dose, enroll additional patients at the current dose, de-escalate to the next lower dose, or declare that the study objectives or stopping rules have been met. The decision will be informed by the mTPI algorithm recommendation to achieve the targeted toxicity interval.

Patients must either experience a DLT during Cycle 1 or receive at least 2 of the 3 planned SYNB1891 injections to be considered evaluable for DLT evaluations and contribute to dose-escalation decisions for a given cohort. Patients who withdraw from the study for any reason other than DLTs prior to receiving the second injection of SYNB1891 in Cycle 1 may be replaced.

Once the RP2D of Arm 2 is determined, up to 20 additional patients may be enrolled in the RP2D cohort to fully characterize the safety profile of SYNB1891 in combination with atezolizumab.

### Definition of Dose-limiting Toxicity

The occurrence of any of the following toxicities during Cycle 1 will be considered a DLT, if assessed by the Investigator to be possibly, probably, or definitely related to study treatment administration:

1. Grade 4 nonhematologic toxicity (not laboratory); 2. Grade 4 hematologic toxicity lasting ≥7 days, except thrombocytopenia: a. Grade 4 thrombocytopenia of any duration, or b. Grade 3 thrombocytopenia associated with clinically significant bleeding; 3. Any nonhematologic AE Grade ≥3 in severity should be considered a DLT, with the following exceptions: Grade 3 fatigue lasting ≤3 days; Grade 3 diarrhea, nausea, or vomiting without use of antiemetics or antidiarrheals per standard of care; Grade 3 rashwithout use of corticosteroids or anti-inflammatory agents per standard of care; 4. Any Grade 3 or Grade 4 nonhematologic laboratory value if: a. Clinically significant medical intervention is required to treat the patient, or b. The abnormality leads to hospitalization, or c. The abnormality persists for >1 week, or d. The abnormality results in a drug-induced liver injury (DILI). Exceptions: clinically nonsignificant, treatable, or reversible laboratory abnormalities including liver function tests, uric acid, etc.; 5. Febrile neutropenia Grade 3 or Grade 4: Grade 3 is defined as absolute neutrophil count (ANC)<1000/mm³ with a single temperature of >38.3° C. (101° F.) or a sustained temperature of ≥38° C. (100.4° F.) for more than 1 hour, or, Grade 4 is defined as ANC<1000/mm³ with a single temperature of >38.3° C. (101° F.) or a sustained temperature of ≥38° C. (100.4° F.) for more than 1 hour, with life-threatening consequences and urgent intervention indicated; 6. Sepsis, severe abscesses and/or ulcerations requiring surgical management; 7. Prolonged delay (>2 weeks) in initiating Cycle 2 due to treatment-related toxicity; 8. Investigator decision to discontinue study treatment for a treatment-related toxicity during Cycle 1; 9. Missing >33% of study drug doses as a result of Grade 3 drug-related AE(s) during the first cycle; and 10. Grade 5 toxicity.

### Intrapatient Dose Modification

### Dosing Delays and Interruptions

Study treatment dosing delays will be permitted in any treatment cycle if unforeseen circumstances prevent the patient from returning to the clinic on the scheduled day(s). A maximum delay of 2 days in Cycle 1 will be permitted. If administration of study treatment is delayed by >2 days in Cycle 1, that dose is considered missed and the patient should receive the next dose at the next visit.

If a patient misses ≥2 SYNB1891 doses in Cycle 1, then the patient will be discontinued permanently from treatment and may be replaced. In Cycle 2 and beyond, if a patient misses the dose of SYNB1891, then the patient may be discontinued permanently from treatment, unless the Investigator's assessment of the benefit/risk suggests otherwise (after consultation with Sponsor). If treatment is permanently discontinued, patients will continue to be followed for safety as indicated for end of treatment and follow-up.

SYNB1891 treatment interruption can occur due to treatment-related toxicity, including Grade 3 or higher infections or local inflammation requiring antibiotics, and the start of a new cycle may not occur until the following criteria have been met:
1. Nonhematologic toxicities have returned to either baseline or Grade ≤1, or at the Investigator's discretion, Grade ≤2 if not considered a safety risk. This condition must be met within 14 days of the initial treatment interruption;
2. Hematologic toxicities: ANC≥1000/mm³ or have returned to baseline levels; platelet count ≥50,000/mm³ or has returned to baseline levels. This condition must be met within 14 days of the initial treatment interruption;
3. If a patient requires treatment with a systemic antibiotic on study, SYNB1891 should be held for the duration of the antibiotic regimen and for 5 half-lives after the last dose of antibiotics. Once the patient is a pyretic after completion of the antibiotic washout period, SYNB1891 may resume.

If the patient cannot meet the above conditions within the specified time frame, then study treatment should be permanently discontinued, unless the Investigator's assessment of the benefit/risk suggests otherwise (after consultation with Sponsor).

Atezolizumab treatment may be temporarily suspended in patients experiencing toxicity considered to be related to study treatment. If corticosteroids are initiated for treatment of immune-mediated toxicity, they must be tapered over ≥1 month to the equivalent of ≤10 mg/day oral prednisone before atezolizumab can be resumed. If atezolizumab is withheld for >12 weeks after event onset, the patient will be discontinued from atezolizumab. However, atezolizumab may be withheld for >12 weeks to allow for patients to taper off corticosteroids prior to resuming treatment. Atezolizumab can be resumed after being withheld for >12 weeks if the Medical Monitor(s) agrees that the patient is likely to derive clinical benefit. Atezolizumab treatment may be suspended for reasons other than toxicity (e.g., surgical procedures) with Medical Monitor approval. The Investigator and the Medical Monitor(s) will determine the acceptable length of treatment interruption.

### Dose Reductions for SYNB1891

No dose reductions of SYNB1891 will be permitted in Cycle 1; any patient who requires such a modification will be permanently discontinued and may be replaced. After Cycle 1, SYNB1891 dose reductions are permitted in approximately 3-fold decrements after consultation with the Sponsor. While no specific dose reductions are required for Grade 1 or 2 treatment-related toxicities, the Investigator should always manage patients according to medical judgment and assessment of risk/benefit for that patient. Patients experiencing recurrent or intolerable Grade 2 toxicity may resume dosing at the next lower dose level once the toxicity has resolved or recovery to Grade 1 or baseline has occurred.

If more than 1 dose reduction is required, then the patient(s) are permanently discontinued from treatment but will be followed for end of treatment and follow-up assessments.

Once a dose has been reduced, the patient should continue to receive that dose for all subsequent doses. In this setting, intrapatient dose escalation is not permitted.

### Dose Reductions for Atezolizumab

No dose reductions of atezolizumab will be permitted in this study.

Adverse Events Associated with SYNB1891

Guidelines for the management of patients who experience AEs considered to be potentially associated with SYNB1891 are provided in Table 11.

TABLE 11
Toxicity Management for Adverse Events Considered Potentially Associated with SYNB1891

| | | Recommended Medical | Premedication and |
|---|---|---|---|
| Event | NCI CTCAE Grade | Management | Subsequent Dosing |
| Cytokine release | Grade 1 | Increase vital sign and | Administer as planned |
| syndrome (may | Fever with or without | oxygensaturation | |
| include associated AEs, | constitutional | monitoring as medically | |
| e.g., fever, tachypnea, | symptoms | indicated until the patient is | |
| headache, tachycardia, | | deemed medically stable in | |
| hypotension, rash, | | the opinion of the | |
| hypoxia) | | Investigator. | |
| | Grade 2 Hypotension | Increase vital sign and | Patient may be |
| | responding to fluids; | oxygensaturation | premedicated 1.5 hours |
| | hypoxia responding to <40% | monitoring as medically | (±30 minutes) prior to |
| | oxygen | indicated until the patient is | study treatment |
| | | deemed medically stable in | administration with |
| | | the opinion of the | oralacetaminophen |
| | | Investigator. | (500 to 1000 mg) or |
| | | Additional appropriate | equivalent dose of |
| | | therapy may include, but is | antipyretics. |
| | | not limited to: | |
| | | IV fluids | |
| | | NSAIDs | |
| | | Acetaminophen | |
| | | Narcotics | |
| | | Oxygen | |
| | | Evaluate for infectious | |
| | | etiologies and treat | |
| | | neutropenia if present. | |
| | Grade 3 | Appropriate therapy may | Discuss with the |
| | Hypotension managed | include, but is not limited | Sponsorprior to the |
| | with one pressor; | to: | resumption ofstudy |
| | hypoxia requiring ≥40% | IV fluids NSAIDs | treatment. Upon |
| | oxygen | Acetaminophen Narcotics | approval by the |
| | | Oxygen Pressors | Sponsor, resume |
| | | Corticosteroids Anti-IL6 | SYNB1891 at a |
| | | (e.g., tocilizumab) | reduced dose upon AE |
| | | Empiric antibiotics Monitor | resolution to baseline |
| | | patients closely; consider | or Grade 1. |
| | | an intensive caresetting | Patient may be |
| | | | premedicated 1.5 hours |
| | | | (±30 minutes) prior to |
| | | | study treatment |
| | | | administration with |
| | | | oralacetaminophen |
| | | | (500 to 1000 mg) or |
| | | | equivalent |
| | | | dose of antipyretics. |
| | Grade 4 | Appropriate therapy may | Permanently |
| | Life-threatening | include, but is not limited | discontinuestudy |
| | consequences; urgent | to: | treatment. |
| | intervention indicated | IV fluids NSAIDs | |
| | | Acetaminophen Narcotics | |
| | | Oxygen Pressors | |
| | | Corticosteroids Anti-IL6 | |
| | | (e.g., tocilizumab) | |
| | | Empiric antibiotics Monitor | |
| | | patients closely; consider | |
| | | an intensive care | |
| | | setting. | |
| Systemic infection | Grade 3 or higher | Appropriate therapy may | Patients who require |
| | | include, but is not limited | systemic antibiotic |
| | | to: | treatment should |
| | | IV fluids NSAIDs | postpone dosing with |
| | | Acetaminophen Narcotics | SYNB1891 for the |
| | | Oxygen Pressors | duration of the |
| | | Empiric antibiotics | antibioticregimen and |
| | | Perform local blood and | for |
| | | urinecultures and | 5 half-lives after the |
| | | sensitivities. | lastdose of antibiotics |
| | | Perform serum qPCR | and until the patient is |
| | | specificfor EcN. | apyretic. |
| Local injection site | Grade 3 or higher | Consider surgical | Permanently |
| reactions (may include | | interventionas needed in | discontinue study |
| associated AEs, e.g., | | addition to and in | treatment. |
| abscess, infection, | | accordance with local | |
| ulceration or necrosis, | | standard of care, which | |
| orsevere tissue | | may include, but is not | |
| damage) | | limited to: | |
| | | NSAIDs | |
| | | Acetaminophen Antibiotics | |

Abbreviations: AE = adverse event; CTCAE = Common Terminology Criteria for Adverse Events; EcN = E. coliNissle; IL = interleukin; IV = intravenous; NCI = National Cancer Institute; NSAID = nonsteroidal anti- inflammatory drug; qPCR = quantitative polymerase chain reaction.

### Duration of Study Participation

The maximum time of study participation for a patient is up to 26 months, including the screening period (up to 28 days), treatment administration period (up to 24 months), and safety follow-up period (30±5 days after the last dose).

### Patient Population

### Number of Patients

Approximately 70 patients are anticipated for enrollment, which provides for 4 to 7 dose-finding cohorts in Arm 1, 1 to 5 cohorts in Arm 2, and replacement of 1 patient per cohort, as well up to 20 additional patients enrolled at the RP2D) in Arm 2. Additional cohorts may be required in either arm to determine the MTD/RP2D.

### Eligibility Criteria

This study will comprise patients with advanced/metastatic solid tumors or lymphoma. Patients will be eligible for enrollment regardless of gender or race/ethnicity.

### Inclusion Criteria

Patients must meet all of the following inclusion criteria to be eligible: (1) Able and willing to voluntarily complete the informed consent process (patient or patient's representative), (2) Adults aged ≥18 years (on the day of signing informed consent) with histologically- or cytologically-confirmed stage III or IV advanced/metastatic solid tumor or lymphoma for which no therapeutic options are available to extend survival or for which the patient is not a candidate for standard-of-care therapy, (3) Eastern Cooperative Oncology Group performance status ≤1, (4) Life expectancy ≥3 months, (5) ≥1 injectable, measurable (≥10 mm in diameter, or ≥15 mm for nodal lesions), eligible lesion as defined by RECIST 1.1 (Eisenhauer et al 2009), iRECIST (Seymour et al 2017), and/or LYRIC (Cheson et al 2016) and as assessed by the Investigator. Eligible lesions must not be located in the thoracic cavity, spleen, pancreas, gastrointestinal tract (liver injection allowed), or cranium and must be amenable to percutaneous injection and away from major blood vessels or neurological structures, (6) Able to provide biopsies for biomarker analysis from injected and (if available)noninjected lesions at baseline and other time points during the study, (7) Oxygen saturation >90% without the use of supplemental oxygen, (9) Adequate cardiac function, defined as follows: (a) Left ventricular ejection fraction (LVEF) >50% by multi-gated acquisition (MUGA)scan or echocardiogram (ECHO) performed within 6 months prior to the first dose of study treatment provided the patient has not received any potential cardiotoxic agents in the intervening period. Symptoms relating to left ventricular dysfunction, cardiac arrhythmia, or cardiac ischemia must all be Grade <1 per NCI CTCAE version 5.0, and (b) QTc interval corrected for heart rate using Fridericia's formula (QTcF)<480 msec at screening.

Other inclusion criteria include:
1. Laboratory values within the following ranges:
   a. Absolute neutrophil count ≥1500/μL
   b. Lymphocyte count ≥500/μL
   c. Platelets ≥100,000/μL without transfusion
   d. Hemoglobin ≥9.0 g/dL (patients may be transfused to meet this criterion)
   e. Estimated glomerular filtration rate (eGFR) >50 mL/min/1.73 m² per the Cockcroft-Gault formula
   f. Total bilirubin ≤1.5×the upper limit of normal (ULN) OR direct bilirubin ≤ULN for participants with total bilirubin levels >1.5×ULN or ≤3×ULN for patients with Gilbert's syndrome
   g. AST, ALT, and alkaline phosphatase ≤2.5×ULN (AST and ALT≤5×ULN for participants with liver metastases and alkaline phosphatase ≤5×ULN for participants with liver or bone metastases)
   h. International normalized ratio (INR) or PT or aPTT≤1.5×ULN unless participant is receiving anticoagulant therapy as long as PT or aPTT is within therapeutic range of intended use of anticoagulants
2. Agree to use an acceptable method of contraception after informed consent, throughout the study, and for 5 months after the last dose of SYNB1891 or atezolizumab.

Additional inclusion criteria that apply only to Arm 2 study participants are as follows:
3. TSH must be within the normal reference range or the patient must be receiving stable thyroid replacement therapy.
4. Patients must have received treatment with an anti-PD-1/L1 monoclonal antibody (mAb) administered either as monotherapy, or in combination with other CPIs or other therapies, if indicated (if CPI therapy is not indicated as a part of standard-of-care therapy, patients may be CPI naïve).

Additional inclusion criteria that apply only to the Arm 2 expansion study participants are as follows:
5. Patients must have progressed on treatment with an anti-PD-1/L1 mAb administered either as monotherapy, or in combination with other CPIs or other therapies, if indicated (if CPI therapy is not indicated as a part of standard-of-care therapy, patients may be CPInaïve).

PD-1/L1 treatment progression is defined by meeting all of the following criteria:
a. Has received at least 2 doses of an approved anti-PD-1/L1 mAb.

Has demonstrated disease progression after PD-1/L1 as defined by RECIST 1.1, iRECIST, and/or LYRIC. The initial evidence of disease progression is to be confirmed by a second assessment no less than 4 weeks from the date of the first documented disease progression, in the absence of rapid clinical progression as determined by the Investigator. Once disease progression is confirmed, the initial date of disease progression documentation will be considered the date of disease progression.
b. Progressive disease has been documented within 12 weeks from the last dose of anti-PD-1/L1 mAb.

### Exclusion Criteria

Patients meeting any of the following criteria are not eligible for study enrollment:
1. Chemotherapy, radiation, or biological cancer therapy within 14 days prior to the first dose of study treatment, or failure of any AEs to recover to Baseline or NCI CTCAE version 5.0 Grade 1, except for any grade of alopecia caused by cancer therapeutics administered >28 days earlier or Grade 2 endocrinopathy from prior checkpoint inhibitor therapy on a stable dose of replacement therapy (e.g., hypothyroidism, adrenal insufficiency).
2. Systemic immunostimulatory agents (including, but not limited to, IFN and IL-2) within 28 days or 5 drug elimination half-lives (whichever is longer) prior to initiation of study treatment. Checkpoint inhibitors are not considered systemic immunostimulatory agents for this criterion and are subject to the washout period listed in exclusion criterion #1.
3. Allogeneic hematopoietic stem cell transplantation that requires current use of immunosuppressors.
4. Receipt of a live vaccine within 90 days prior to the first dose of study treatment or anticipation of a need for such a vaccine during treatment.
5. Receipt of antibiotics within 7 days prior to the first dose of study treatment.
6. Participation in a study of an investigational agent and receipt of study therapy or use of an investigational device within 28 days prior to the first dose of study treatment.
7. Diagnosed immunodeficiency or current use of chronic systemic steroid therapy in excess of replacement doses (prednisone ≤10 mg/day or equivalent is acceptable) or any other form of immunosuppressive medication within 7 days prior to the first dose of study treatment.
8. For injection and biopsy of visceral (deep) lesions: patients must not be on long-acting antiplatelet agents such as aspirin or clopidogrel or on therapeutic doses of anticoagulants, with the exception of patients receiving a preventative dose of low molecular weight heparin. In patients receiving preventative low molecular weight heparin, treatment must be stopped 24 hours before the intratumoral injection and resumed again 24 hours after the injection (Marabelle et al 2018).
9. Previous or concurrent malignancy, with the exception of:
   a. Adequately treated basal or squamous cell carcinoma, in situ carcinoma of the cervix, or ductal carcinoma in situ of the breast;
   b. Localized prostate cancer definitively treated with surgery or radiation or stable on hormone therapy;
   c. Other cancer from which the patient has been disease free for at least 2 years.
10. Clinically active central nervous system (CNS) metastases and/or carcinomatous meningitis (treated brain metastases are permitted if radiologically stable for ≥28 days prior to the first dose of study treatment).
11. Allergy to antibiotics that precludes treatment for infection with E. coli Nissle 1917.
12. Hepatitis B or C infection(s) unless screening tests indicate a negative viral load, and/or human immunodeficiency virus (HIV) infection unless screening tests indicate a negative or below the limit of quantitation viral load.
13. Known active tuberculosis.
14. Grade 3 or higher infection according to NCI CTCAE within 28 days prior to initiation of study treatment, including, but not limited to, hospitalization for complications of infection, bacteremia, or severe pneumonia.
15. Failure to fully recover from the effects of major surgery. Surgeries that required general anesthesia must be completed >14 days before the first dose of study drug. Surgery requiring regional/epidural anesthesia must be completed >72 hours before the first dose of study treatment and participants should be recovered.
16. Heart failure of New York Heart Association Class 3 or greater, restrictive cardiomyopathy, or unstable angina or recent myocardial infarction within 3 months prior to the first dose of study treatment.
17. Any other medical condition that might confound the results of the study, interfere with the participant's participation, or is not in the best interest of the participant, in the opinion of the treating Investigator.
18. Pregnant or breastfeeding or anticipated conception or fathering of children within the projected duration of the study and for 5 months after the last dose of SYNB1891 or atezolizumab.

Additional exclusion criteria that apply only to Arm 2 study participants are as follows:
19. History of immune-mediated AEs on prior PD-1/PD-L1 therapies requiring discontinuation or immunosuppressor therapy, excluding endocrinopathies.
20. Active or history of underlying autoimmune disease or immune deficiency, including, but not limited to, myasthenia gravis, myositis, autoimmune hepatitis, systemic lupus erythematosus, rheumatoid arthritis, inflammatory bowel disease, antiphospholipid antibody syndrome, Wegener granulomatosis, Sjögren syndrome, Guillain-Barré syndrome, or multiple sclerosis, with the following exceptions:
   a. Patients with a history of autoimmune-related hypothyroidism who are on thyroid replacement hormone are eligible for the study.
   b. Patients with controlled Type 1 diabetes mellitus who are on an insulin regimen are eligible for the study.
   c. Patients with eczema, psoriasis, lichen simplex chronicus, or vitiligo with dermatologic manifestations only (e.g., patients with psoriatic arthritis are excluded) are eligible for the study provided all of following conditions are met:
      1. Rash must cover ≤10% of body surface area;
      2. Disease is well controlled at baseline and requires only low-potency topical corticosteroids;
      3. No occurrence of acute exacerbations of the underlying condition requiring psoralen plus ultraviolet A radiation, methotrexate, retinoids, biologic agents, oral calcineurin inhibitors, or high potency or oral corticosteroids within the previous 12 months.
21. History of a Grade ≥3 allergic anaphylactic reaction following treatment with a PD-1 mAb or to chimeric, human, or humanized antibodies or fusion proteins.
22. History of idiopathic pulmonary fibrosis, organizing pneumonia (e.g., bronchiolitis obliterans), drug-induced pneumonitis requiring treatment, or idiopathic pneumonitis, orevidence of active pneumonitis.
23. Known hypersensitivity to Chinese hamster ovary cell products or any component of the atezolizumab formulation.

1. A method of treating a human subject with cancer, the method comprising:
administering to the human subject a recombinant microorganism capable of expressing a stimulator of interferon gene (STING) agonist,
wherein a level of one or more interferon-stimulated genes, one or more T cell function genes, one or more chemokine genes, one or more cytokine genes, and/or one or more serum cytokines are upregulated in the human subject after administration, as compared to a control level of the one or more interferon-stimulated genes, one or more T cell function genes, one or more chemokine genes, one or more cytokine genes, and/or one or more serum cytokines.

2. The method of claim 1, wherein the one or more interferon-stimulated genes are selected from the group consisting of ISG15, IFIT1, and IFIT2.

3. The method of claim 1 or claim 2, wherein the one or more T cell function genes are selected from the group consisting of GZMA, CD4, CD8, and PD-L2.

4. The method of any one of claims 1-3, wherein the one or more chemokines and/or cytokine genes are selected from the group consisting of CXCL9, CXCL10, TNFRS1B, and TNFSF10.

5. The method of any one of claims 1-4, wherein the level of the one or more interferon-stimulated genes, one or more T cell function genes, one or more chemokine genes, and/or one or more cytokine genes in the subject after administration is upregulated by about 2-fold, about 3-fold, or about 4-fold, as compared to the control level.

6. The method of any one of claims 1-5, wherein the one or more serum cytokines are selected from the group consisting of IL-6, TNFα, IFNγ, and IL-1Ra.

7. The method of claim 6, wherein the level of the one or more serum cytokines is about 2-fold, about 5-fold, about 10-fold, or about 20-fold higher than the control level of the one or more serum cytokines.

8. The method of any one of the previous claims, wherein the control level is (i) a level of the one or more interferon-stimulated genes, one or more T cell function genes, one or more chemokine genes, one or more cytokine genes, and/or one or more serum cytokines in the human subject prior to administration, or (ii) a level of the one or more interferon-stimulated genes, one or more T cell function genes, one or more chemokine genes, one or more cytokine genes, and/or one or more serum cytokines in a control human subject or population of control human subjects who has not been administered the recombinant microorganism.

9. The method of any one of the previous claims, wherein the recombinant microorganism is administered to the subject at a dose of about 1×10⁶, about 3×10⁶, about 1×10⁷, about 3×10⁷, about 1×10⁸, about 3×10⁸, or about 1×10⁹ live cells.

10. The method of any one of claims 1-8, wherein the recombinant microorganism is administered to the subject at a dose of about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, and from about 3×10⁸ to about 1×10⁹ live cells.

11. The method of any one of the previous claims, wherein the recombinant microorganism is administered to the subject once weekly, once every two weeks, or once every three weeks.

12. The method of claim 11, wherein the recombinant microorganism is administered to the subject once weekly.

13. The method of claim 11, wherein the recombinant microorganism is administered to the subject once every three weeks.

14. The method of any one of the previous claims, wherein the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months.

15. The method of claim 14, wherein the recombinant microorganism is administered for at least 24 months.

16. The method of any one of the previous claims, wherein the cancer is a solid tumor, a lymphoma, melanoma, liposarcoma, sarcoma, small cell lung cancer, squamous cell carcinoma, or chondrosarcoma.

17. The method of any one of the previous claims, wherein the STING agonist is c-diAMP, c-GAMP, or c-diGMP.

18. The method of claim 17, wherein the STING agonist is c-diAMP.

19. The method of any one of the previous claims, wherein the recombinant microorganism expresses at least one non-native gene sequence encoding an enzyme capable of producing the STING agonist.

20. The method of claim 19, wherein the at least one non-native gene sequence is dacA or cGAS.

21. The method of claim 19 or claim 20, wherein the at least one non-native gene sequence is integrated into a chromosome of the microorganism.

22. The method of claim 19 or claim 20, wherein the at least one non-native gene sequence is present on a plasmid in the microorganism.

23. The method of claim 21 or claim 22, wherein the at least one non-native gene sequence encoding the enzyme capable of producing the STING agonist is operably linked to a constitutive promoter.

24. The method of claim 21 or claim 22, wherein the at least one non-native gene sequence encoding the enzyme capable of producing the STING agonist is operably linked to an inducible promoter.

25. The method of claim 24, wherein inducible promoter is induced by low-oxygen or anaerobic conditions, by the hypoxic environment of a tumor, or temperature.

26. The method of any one of the preceding claims, wherein the recombinant microorganism further comprises one or more auxotrophies and/or one or more endogenous phage deletions.

27. The method of claim 26, wherein the recombinant microorganism is an auxotroph in dapA, thyA, or both dapA and thyA.

28. The method of any one of the preceding claims, wherein the recombinant microorganism is non-pathogenic to the subject.

29. The method of any one of the preceding claims, wherein the recombinant microorganism is Escherichia coli Nissle.

30. The method of any one of the previous claims, further comprising selecting a subject who would benefit from an increase in the level of the one or more interferon-stimulated genes, one or more T cell function genes, one or more chemokine genes, one or more cytokine genes, and/or one or more serum cytokines.

31. The method of any one of the previous claims, further comprising isolating a sample from the subject after administration and measuring the level of the one or more interferon-stimulated genes, one or more T cell function genes, one or more chemokine genes, one or more cytokine genes, and/or one or more serum cytokines.

32. The method of any one of the previous claims, further comprising isolating a sample from the subject prior to administration and measuring the level of the one or more interferon-stimulated genes, one or more T cell function genes, one or more chemokine genes, one or more cytokine genes, and/or one or more serum cytokines.

33. The method of claim 31 or claim 32, wherein the sample is a tumor biopsy and/or a serum sample.

34. The method of any one of the previous claims, wherein the administering is intratumoral injection.

35. A method of increasing the number of T cells present in a tumor or a cancer in a human subject, the method comprising administering to the human subject a recombinant microorganism capable of expressing a stimulator of interferon gene (STING) agonist, wherein the number of T cells present in the tumor or cancer is increased by at least about 25%, about 50%, about 75%, about 100%, about 1.5-fold, about 2-fold, about 2.5-fold or about 3-fold after administration of the recombinant microorganism as compared to a number of T cells present in the tumor or cancer prior to administration of the recombinant microorganism.

36. The method of claim 35, wherein the T cells present in the tumor after administration are CD4+, CD8+, CD11c+/MHCII+, CD11c+/PD-L1+, CD4+/Ki67+, CD8+/Ki67+, CD8+/GrnzB+, CD11c+/CD8+/MHCII+, CD11c+, MHCII+, PD-LI+, or a combination thereof.

37. A method of treating a human subject having a tumor or a cancer, the method comprising: selecting a human subject that would benefit from an increase in the number of T cells present in the tumor, and
administering to the human subject a recombinant microorganism capable of expressing a stimulator of interferon gene (STING) agonist,
wherein the number of T cells present in the tumor is increased by at least about 25%, about 50%, about 75%, about 100%, about 1.5-fold, about 2-fold, about 2.5-fold or about 3-fold after administration of the recombinant microorganism as compared to a number of T cells present in the tumor prior to administration of the recombinant microorganism.

38. The method of claim 37, wherein the T cells present in the tumor after administration are CD4+, CD8+, CD11c+/MHCII+, CD11c+/PD-L1+, CD4+/Ki67+, CD8+/Ki67+, CD8+/GrnzB+, CD11c+/CD8+/MHCII+, CD11c+, MHCII+, PD-LI+, or a combination thereof.

39. The method of any one of claims 35-38, wherein the tumor or cancer is stable or regressing after administration.

40. A method of increasing survival of a human subject suffering from cancer or having a tumor, the method comprising administering to the human subject a recombinant microorganism capable of expressing a stimulator of interferon gene (STING) agonist, wherein the number of T cells present in the tumor or cancer is increased by at least about 25%, about 50%, about 75%, about 100%, about 1.5-fold, about 2-fold, about 2.5-fold or about 3-fold after administration of the recombinant microorganism as compared to a number of T cells present in the tumor or cancer prior to administration of the recombinant microorganism, wherein the cancer and/or tumor is stable or regressing after administration.

41. The method of claim 40, wherein the T cells present in the tumor after administration are CD4+, CD8+, CD11c+/MHCII+, CD11c+/PD-L1+, CD4+/Ki67+, CD8+/Ki67+, CD8+/GrnzB+, CD11c+/CD8+/MHCII+, CD11c+, MHCII+, PD-LI+, or a combination thereof.

42. The method of any one of claims 35-41, wherein the recombinant microorganism is administered to the subject at a dose of about 1×10⁶, about 3×10⁶, about 1×10⁷, about 3×10⁷, about 1×10⁸, about 3×10⁸, or about 1×10⁹ live cells.

43. The method of any one of claims 35-41, wherein the recombinant microorganism is administered to the subject at a dose of about 1×10⁶ to about 3×10⁶, from about 3×10⁶ to about 1×10⁷, from about 1×10⁷ to about 3×10⁷, from about 3×10⁷ to about 1×10⁸, from about 1×10⁸ to about 3×10⁸, and from about 3×10⁸ to about 1×10⁹ live cells.

44. The method of any one of claims 35-43, wherein the recombinant microorganism is administered to the subject once weekly, once every two weeks, or once every three weeks.

45. The method of claim 44, wherein the recombinant microorganism is administered to the subject once weekly.

46. The method of claim 44, wherein the recombinant microorganism is administered to the subject once every three weeks.

47. The method of any one of claims 35-46, wherein the recombinant microorganism is administered for at least 3 weeks, at least 6 weeks, at least 2 months, at least 4 months, at least 6 months, at least 8 months, at least 10 months, at least 12 months, at least 14 months, at least 16 months, at least 18 months, at least 20 months, at least 22 months, or at least 24 months.

48. The method of claim 47, wherein the recombinant microorganism is administered for at least 24 months.

49. The method of any one of claims 35-47, wherein the STING agonist is c-diAMP, c-GAMP, or c-diGMP.

50. The method of any one of claims 35-49, wherein the recombinant microorganism expresses at least one non-native gene sequence encoding an enzyme capable of producing the STING agonist.

51. The method of claim 50, wherein the at least one non-native gene sequence is dacA or cGAS.

52. The method of any one of claims 35-51, wherein the cancer or tumor is a solid tumor, a lymphoma, melanoma, liposarcoma, sarcoma, small cell lung cancer, squamous cell carcinoma, or chondrosarcoma.

53. The method of any one of the previous claims, wherein the tumor responds to treatment resulting in a decrease of at least 5%, at least 10%, at least 20%, at least 25%, or at least 30% of a Response Evaluation Criteria in Solid Tumours (RECIST) when compared to the RECIST prior to treatment.

54. The method of any one of the previous claims, further comprising administering an additional therapeutic agent, optionally wherein the additional therapeutic agent is an anti-PD1 antibody or an anti-PDL1 antibody.

55. The method of claim 54, wherein the anti-PDL1 antibody is atezolizumab.

56. The method of any one of the previous claims, wherein the microorganism is SYNB1891.

57. The method of claim 54, wherein SYNB1891 comprises the genotype ΔthyA-ΔdapA-ΔΦ-P_{furS}-dacA.
